(19) United States
(12) Patent Application Publication
(10) Pub. No.: US 2011/0053910 A1
(43) Pub. Date: Mar. 3, 2011

(54) 2 -HETEROCYCLYLOXYBENZOYL AMINO HETEROCYCLYL COMPOUNDS AS MODULATORS OF GLUCOKINASE FOR THE TREATMENT OF TYPE 2 DIABETES

(76) Inventors: Darren McKerrecher, Cheshire (GB); Kurt Gordon Pike, Cheshire (GB); Michael James Waring, Cheshire (GB)

(21) Appl. No.: 11/995,076

(22) PCT Filed: Jul. 3, 2006
(86) PCT No.: PCT/GB2006/002460

§ 371 Date: Aug. 1, 2008

(30) Foreign Application Priority Data
Jul. 9, 2005 (GB) 0514175.9
Aug. 9, 2005 (GB) 0516299.5
Nov. 24, 2005 (GB) 0523863.9

(51) Int. Cl. A61K 31/553; C07D 417/12; A61K 31/428; C07D 498/04; C07D 513/04; A61K 31/55; A61K 31/5365; A61P 3/10

(52) U.S. Cl. 514/211.06; 548/171; 514/367; 540/491; 540/521; 514/212.06; 544/91; 514/230.5

(57) ABSTRACT

Compounds of formula (I)

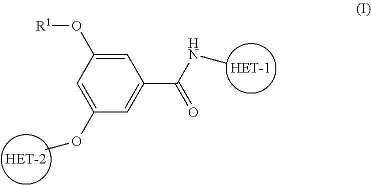

wherein R¹, HET-1 and HET-2 are as described in the specification,
and their salts, are activators of glucokinase (GLK) and are thereby useful in the treatment of, for example, type 2 diabetes. Processes for preparing compounds of formula (I) are also described.

The present invention relates to a group of benzoyl amino heterocyclyl compounds which are useful in the treatment or prevention of a disease or medical condition mediated through glucokinase (GLK or GK), leading to a decreased glucose threshold for insulin secretion. In addition the compounds are predicted to lower blood glucose by increasing hepatic glucose uptake. Such compounds may have utility in the treatment of Type 2 diabetes and obesity. The invention also relates to pharmaceutical compositions comprising said compounds and to methods of treatment of diseases mediated by GLK using said compounds.

In the pancreatic β-cell and liver parenchymal cells the main plasma membrane glucose transporter is GLUT2. Under physiological glucose concentrations the rate at which GLUT2 transports glucose across the membrane is not rate limiting to the overall rate of glucose uptake in these cells. The rate of glucose uptake is limited by the rate of phosphorylation of glucose to glucose-6-phosphate (G-6-P) which is catalysed by glucokinase (GLK) [1]. GLK has a high (6-10 mM) Km for glucose and is not inhibited by physiological concentrations of G-6-P [1]. GLK expression is limited to a few tissues and cell types, most notably pancreatic β-cells and liver cells (hepatocytes) [1]. In these cells GLK activity is rate limiting for glucose utilisation and therefore regulates the extent of glucose induced insulin secretion and hepatic glycogen synthesis. These processes are critical in the maintenance of whole body glucose homeostasis and both are dysfunctional in diabetes [2].

In one sub-type of diabetes, Maturity-Onset Diabetes of the Young Type 2 (MODY-2), the diabetes is caused by GLK loss of function mutations [3, 4]. Hyperglycaemia in MODY-2 patients results from defective glucose utilisation in both the pancreas and liver [5]. Defective glucose utilisation in the pancreas of MODY-2 patients results in a raised threshold for glucose stimulated insulin secretion. Conversely, rare activating mutations of GLK reduce this threshold resulting in familial hyperinsulinism [6, 6a, 7]. In addition to the reduced GLK activity observed in MODY-2 diabetics, hepatic glucokinase activity is also decreased in type 2 diabetics [8]. Importantly, global or liver selective overexpression of GLK prevents or reverses the development of the diabetic phenotype in both dietary and genetic models of the disease [9-12]. Moreover, acute treatment of type 2 diabetics with fructose improves glucose tolerance through stimulation of hepatic glucose utilisation [13]. This effect is believed to be mediated through a fructose induced increase in cytosolic GLK activity in the hepatocyte by the mechanism described below [13].

Hepatic GLK activity is inhibited through association with GLK regulatory protein (GLKRP). The GLK/GLKRP complex is stabilised by fructose-6-phosphate (F6P) binding to the GLKRP and destabilised by displacement of this sugar phosphate by fructose-1-phosphate (F1P). F1P is generated by fructokinase mediated phosphorylation of dietary fructose. Consequently, GLK/GLKRP complex integrity and hepatic GLK activity is regulated in a nutritionally dependent manner as F6P is dominant in the post-absorptive state whereas F1P predominates in the post-prandial state. In contrast to the hepatocyte, the pancreatic β-cell expresses GLK in the absence of GLKRP. Therefore, β-cell GLK activity is regulated extensively by the availability of its substrate, glucose. Small molecules may activate GLK either directly or through destabilising the GLK/GLKRP complex. The former class of compounds are predicted to stimulate glucose utilisation in both the liver and the pancreas whereas the latter are predicted to act selectively in the liver. However, compounds with either profile are predicted to be of therapeutic benefit in treating Type 2 diabetes as this disease is characterised by defective glucose utilisation in both tissues.

GLK, GLKRP and the K_{ATP} channel are expressed in neurones of the hypothalamus, a region of the brain that is important in the regulation of energy balance and the control of food intake [14-18]. These neurones have been shown to express orectic and anorectic neuropeptides [15, 19, 20] and have been assumed to be the glucose-sensing neurones within the hypothalamus that are either inhibited or excited by changes in ambient glucose concentrations [17, 19, 21, 22]. The ability of these neurones to sense changes in glucose levels is defective in a variety of genetic and experimentally induced models of obesity [23-28]. Intracerebroventricular (icy) infusion of glucose analogues, that are competitive inhibitors of glucokinase, stimulate food intake in lean rats [29, 30]. In contrast, icy infusion of glucose suppresses feeding [31]. Thus, small molecule activators of GLK may decrease food intake and weight gain through central effects on GLK. Therefore, GLK activators may be of therapeutic use in treating eating disorders, including obesity, in addition to diabetes. The hypothalamic effects will be additive or synergistic to the effects of the same compounds acting in the liver and/or pancreas in normalising glucose homeostasis, for the treatment of Type 2 diabetes. Thus the GLK/GLKRP system can be described as a potential “Diabesity” target (of benefit in both Diabetes and Obesity).

GLK is also expressed in specific entero-endocrine cells where it is believed to control the glucose sensitive secretion of the incretin peptides GIP (glucose-dependent insulinotropic polypeptide) and GLP-1 (Glucagon-Like Peptide-1) from gut K-cells and L-cells respectively (32, 33, 34). Therefore, small molecule activators of GLK may have additional beneficial effects on insulin secretion, b-cell function and survival and body weight as a consequence of stimulating GIP and GLP-1 secretion from these entero-endocrine cells.

In WO00/58293 and WO01/44216 (Roche), a series of benzylcarbamoyl compounds are described as glucokinase activators. The mechanism by which such compounds activate GLK is assessed by measuring the direct effect of such compounds in an assay in which GLK activity is linked to NADH production, which in turn is measured optically—see details of the in vitro assay described hereinafter. Compounds of the present invention may activate GLK directly or may activate GLK by inhibiting the interaction of GLKRP with GLK.

Further GLK activators have been described in WO03/095438 (substituted phenylacetamides, Roche), WO03/055482 (carboxamide and sulphonamide derivatives, Novo Nordisk), WO2004/002481 (arylcarbonyl derivatives, Novo Nordisk), and in WO03/080585 (amino-substituted benzoylaminoheterocycles, Banyu).

Our International application Number: WO03/000267 describes a group of benzoyl amino pyridyl carboxylic acids which are activators of the enzyme glucokinase (GLK).

Our International application Number: WO03/015774 describes compounds of the Formula (A):

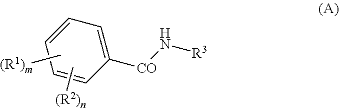

wherein R³ is a substituted heterocycle other than a carboxylic acid substituted pyridyl.

International application WO2004/076420 (Banyu) describes compounds which are generally a subset of those described in WO03/015774, wherein for example R¹ is an (substituted) alkyl ether and R² is (substituted) phenoxy.

We have surprisingly found a small group of compounds, generally a selected subgroup of those described in WO 03/015774, which have generally superior potency for the GLK enzyme, and more advantageous physical properties, including, for example, higher aqueous solubility, higher permeability, and/or lower plasma protein binding. Consequently, such compounds having a balance of these properties would be expected to display higher plasma free drug levels and superior in vivo efficacy after oral dosing as determined, for example, by activity in Oral Glucose Tolerance Tests (OGTTs). Therefore this group of compounds would be expected to provide superior oral exposure at a lower dose and thereby be particularly suitable for use in the treatment or prevention of a disease or medical condition mediated through GLK. Furthermore, the compounds of the invention may have favourable metabolic profiles and/or toxicity profiles. The compounds of the invention may also have superior potency and/or advantageous physical properties (as described above) and/or favourable toxicity profiles and/or favourable metabolic profiles in comparison with other GLK activators known in the art, as well as those described in WO 03/015774.

Thus, according to the first aspect of the invention there is provided a compound of Formula (I):

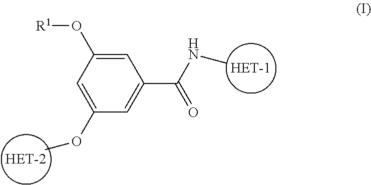

wherein:
R¹ is selected from isopropyl, but-2-yl, 1,1,1-trifluoroprop-2-yl, 1,3-difluoroprop-2-yl, but-1-yn-3-yl, 1-hydroxyprop-2-yl, 2-hydroxybut-3-yl, 1-hydroxybut-2-yl, tetrahydrofuryl, tetrahydropyranyl, 1-methoxyprop-2-yl, 1-methoxybut-2-yl, 2-hydroxyprop-1-yl, 2-methoxyprop-1-yl, 2-hydroxybut-1-yl, 2-methoxybut-1-yl, 1-fluoromethoxyprop-2-yl, 1,1-difluoromethoxyprop-2-yl and 1-trifluoromethoxyprop-2-yl;
HET-1 is a 5- or 6-membered, C-linked heteroaryl ring containing a nitrogen atom in the 2-position and optionally 1 or 2 further ring heteroatoms independently selected from O, N and S; which ring is optionally substituted on any nitrogen atom by a substituent selected from R⁷ and/or on any available carbon atom by 1 or 2 substituents independently selected from R⁶;
HET-2 is a heterocyclic ring system comprising a Ring A (which is bonded to the linking ether oxygen) and a Ring B which is fused to Ring A;
wherein Ring A is a 5- or 6-membered heteroaryl ring, and Ring A is optionally substituted with a substituent selected from R⁴;
Ring B is phenyl or Ring B is a 5-7 membered heterocyclic ring, containing 1, 2 or 3 ring hetereoatoms independently selected from O, S and N (provided that there are no O—O, S—O or S—S bonds within the ring), wherein any ring carbon or sulfur atom may optionally be oxidised and wherein Ring B is optionally substituted on any nitrogen atom by a substituent selected from R² and/or on any available carbon atom by 1 or 2 substituents independently selected from R³;
R² is selected from (1-4C)alkyl, (3-6C)cycloalkyl, benzyl, (1-4C)alkylcarbonyl, (1-4C)alkylsulphonyl, hydroxy(1-4C)alkyl and (1-4C)alkoxy(1-4C)alkyl;
R³ is selected from (1-4C)alkyl, (3-6C)cycloalkyl, (1-4C)alkoxy, hydroxy, fluoro and chloro;
when R⁴ is a substituent on carbon, it is selected from fluoro and chloro;
when R⁴ is a substituent on nitrogen it is selected from (1-4C)alkyl, (3-6C)cycloalkyl, benzyl, (1-4C)alkylcarbonyl, (1-4C)alkylsulphonyl, hydroxy(1-4C)alkyl and (1-4C)alkoxy(1-4C)alkyl;
R⁶ is independently selected from (1-4C)alkyl, halo, hydroxy(1-4C)alkyl, (1-4C)alkoxy(1-4C)alkyl, (1-4C)alkyl S(O)p(1-4C)alkyl, amino(1-4C)alkyl, (1-4C)alkylamino(1-4C)alkyl and di(1-4C)alkylamino(1-4C)alkyl;
R⁷ is independently selected from (1-4C)alkyl, hydroxy(1-4C)alkyl, (1-4C)alkoxy(1-4C)alkyl, (1-4C)alkyl S(O)p(1-4C)alkyl, amino(1-4C)alkyl, (1-4C)allylamino(1-4C)alkyl and di(1-4C)allylamino(1-4C)alkyl;
p is (independently at each occurrence) 0, 1 or 2;
or a salt thereof.

In another aspect of the invention there is provided a compound of formula (I) as hereinbefore defined, wherein

R¹ is selected from isopropyl, but-2-yl, 1,1,1-trifluoroprop-2-yl, 1,3-difluoroprop-2-yl, but-1-yn-3-yl, 1-hydroxyprop-2-yl, 2-hydroxybut-3-yl, 1-hydroxybut-2-yl, tetrahydrofuryl, tetrahydropyranyl, 1-methoxyprop-2-yl, 1-methoxybut-2-yl, 1-fluoromethoxyprop-2-yl, 1,1-difluoromethoxyprop-2-yl and 1-trifluoromethoxyprop-2-yl;
or a salt thereof.

It will be understood that substitution of a nitrogen in Ring A by a substituent R⁴ may not lead to quaternisation of that nitrogen.

It will be understood that Ring B may be an unsaturated (including aromatic where possible), partially or fully saturated ring system.

It will be understood that R² can be present on any nitrogen atom, so if there is more than one nitrogen atom in Ring B, any or all may be substituted by an R² group, which may be the same or different, provided that the substituted nitrogen is not thereby quaternatised.

It will be understood that R³ can be present on any or all available carbon atoms in Ring B; each carbon atom can be substituted with 1 or 2 R³ groups which may be the same or different, provided the structure thereby formed is stable (so, for example, it is not intended to cover gem-dihydroxy substitution).

Compounds of Formula (I) may form salts which are within the ambit of the invention. Pharmaceutically acceptable salts are preferred although other salts may be useful in, for example, isolating or purifying compounds.

In another aspect, the invention relates to compounds of formula (I) as hereinabove defined or to a pharmaceutically acceptable salt.

In another aspect, the invention relates to compounds of formula (I) as hereinabove defined or to a pro-drug thereof. Suitable examples of pro-drugs of compounds of formula (I) are in-vivo hydrolysable esters of compounds of formula (I). Therefore in another aspect, the invention relates to compounds of formula (I) as hereinabove defined or to an in-vivo hydrolysable ester thereof.

In this specification the generic term “alkyl” includes both straight-chain and branched-chain alkyl groups. However references to individual alkyl groups such as “propyl” are specific for the straight chain version only and references to individual branched-chain alkyl groups such as t-butyl are specific for the branched chain version only. For example, “(1-4C)alkyl” includes methyl, ethyl, propyl, isopropyl and t-butyl. An analogous convention applies to other generic terms.

For the avoidance of doubt, reference to the group HET-1 containing a nitrogen in the 2-position, is intended to refer to the 2-position relative to the amide nitrogen atom to which the group is attached. For example, HET-1 encompasses but is not limited to the following structures:

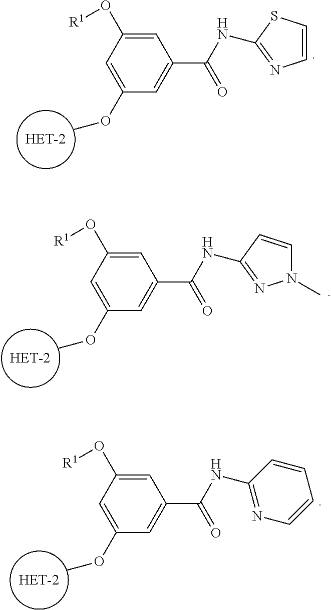

Suitable examples of HET-1 as a 5- or 6-membered, C-linked heteroaryl ring as hereinbefore defined, include thiazolyl, isothiazolyl, thiadiazolyl, pyridyl, pyrazinyl, pyridazinyl, pyrazolyl, imidazolyl, pyrimidinyl, oxazolyl, isoxazolyl, oxadiazolyl and triazolyl.

It will be understood that the group —O-HET-2 may be drawn as:

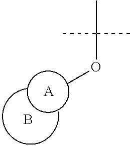

Suitable values for the bicyclic system HET-2 formed by ring A fused to Ring B include those where Ring B is pyridyl, pyrazinyl, pyrimidinyl, piperidinyl, piperazinyl, homopiperazinyl, morpholinyl, homomorpholinyl, thiomorpholinyl, homothiomorpholinyl, oxathianyl, homooxathianyl, furyl, thienyl, pyrrolyl, pyrrolidinyl, 1,3-dioxolanyl, oxazolyl, thiazolyl, imidazolyl, imidazolidinyl, pyrazolyl, isoxazolyl, isothiazolyl, and pyranyl. Further suitable values include such ring systems where one or more carbon atoms in the HET-2 ring have been oxidised to a carbonyl group, and/or where one or more sulfur atoms in the HET-2 ring have been oxidised to an S(O) or S(O)₂ group. A further suitable value for Ring B is phenyl.

Suitable values for Ring A are furyl, thienyl, pyrrolyl, pyrrolidinyl, oxazolyl, thiazolyl, imidazolyl, pyrazolyl, isoxazolyl, isothiazolyl, pyridyl, pyrazinyl, pyrimidinyl and pyridazinyl. Further suitable values for Ring A are thiazolyl, pyridyl, pyrazinyl, pyrimidinyl and pyridazinyl. Still further suitable values for Ring A are thiazolyl and pyridyl.

For example, HET-2 may suitably be selected from the structures below (which may optionally be substituted as hereinbefore defined):

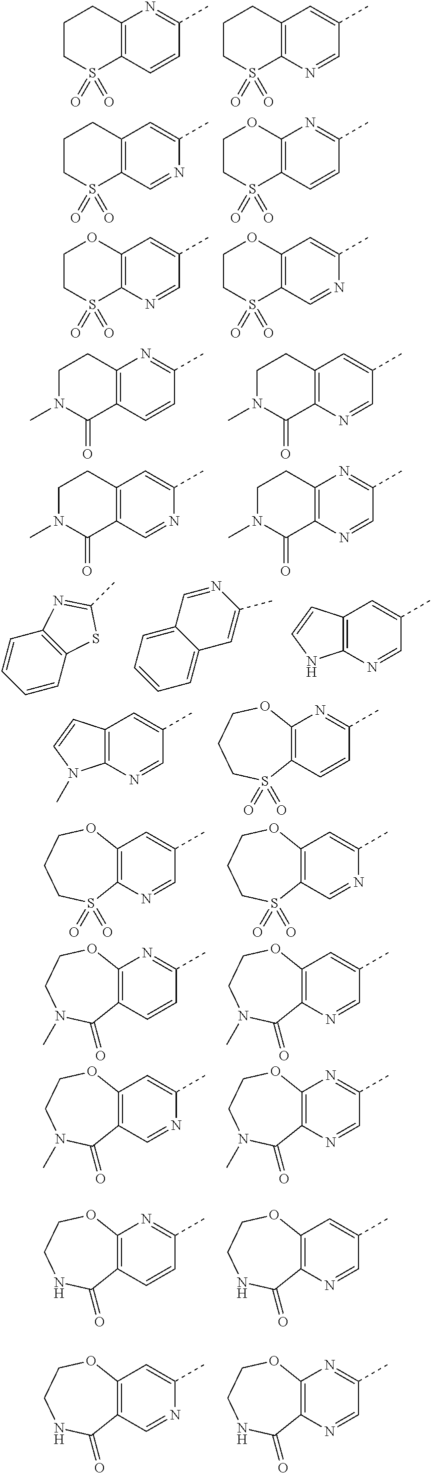

In a further aspect, suitable values for HET-2 are ring systems where Ring B is a 7-membered ring, for example:

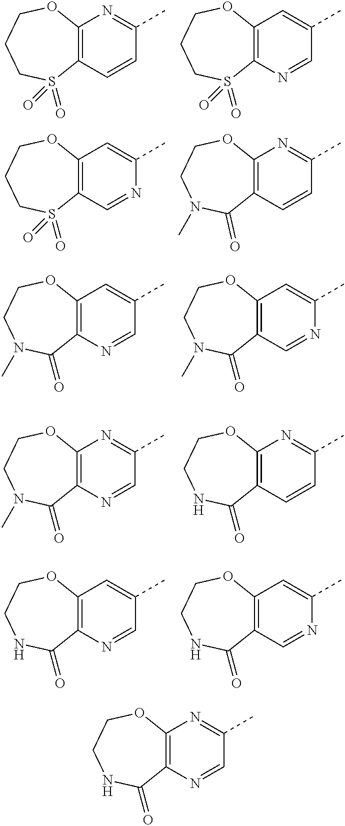

Further suitable values for HET-2 include the following formulae A to F, wherein each R^{2a} is independently hydrogen or is R² as hereinbefore defined, each R^{3a} is independently hydrogen or is R³ as hereinbefore defined, each R^{4a} is independently hydrogen or is R⁴ as hereinbefore defined:

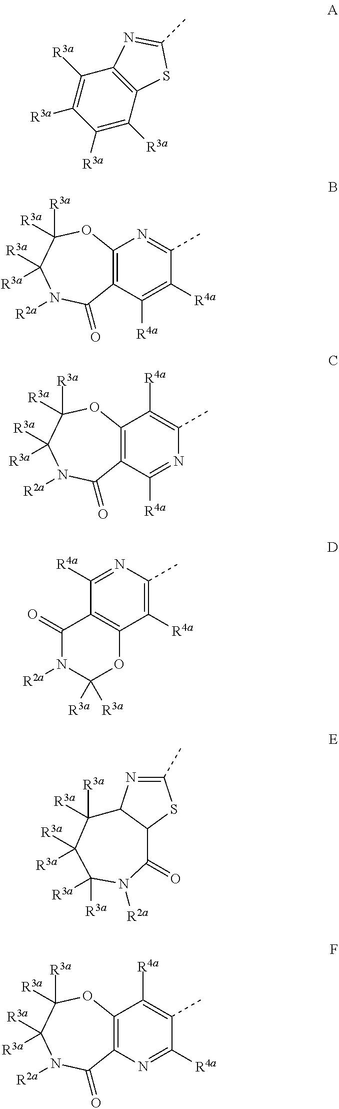

Further suitable values for HET-2 include formulae G to P as follows:

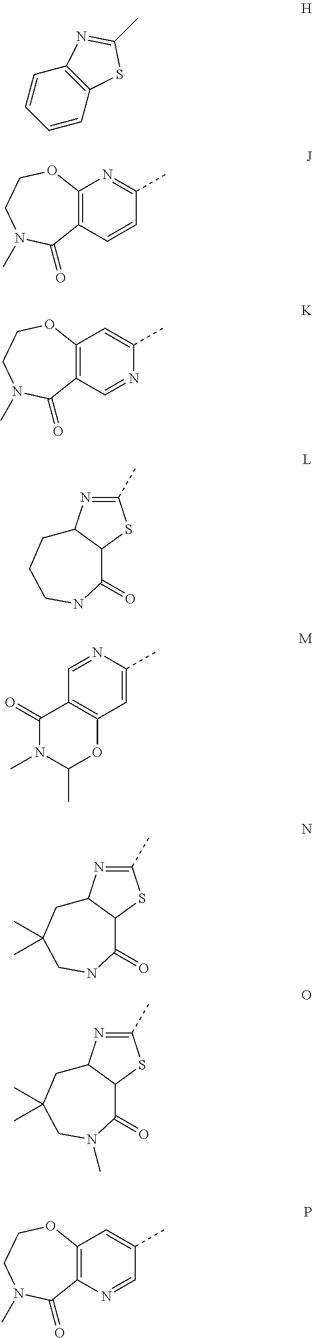

It will be appreciated that the bicyclic ring systems shown above are to illustrate the definitions of Ring B and may be applied to any of the possible values for Ring A, even if not shown above

It will be appreciated that, where definitions of heterocyclyl groups HET-1 and HET-2 encompass heteroaryl rings which may be substituted on nitrogen, such substitution may not result in charged quaternary nitrogen atoms or unstable structures. It will be appreciated that the definitions of HET-1 and HET-2 are not intended to include any O—O, O—S or S—S bonds. It will be appreciated that the definitions of HET-1 and HET-2 are not intended to include unstable structures.

Examples of (1-4C)alkyl include methyl, ethyl, propyl, isopropyl, butyl and tert-butyl; examples of (3-6C)cycloalkyl include cyclopropyl, cyclobutyl, cyclopentyl and cyclohexyl; examples of halo include fluoro, chloro, bromo and iodo; examples of hydroxy(1-4C)alkyl include hydroxymethyl, 1-hydroxyethyl, 2-hydroxyethyl, 2-hydroxypropyl, 3-hydroxypropyl, 1-hydroxyisopropyl and 4-hydroxybutyl; examples of (1-4C)alkoxy(1-4C)alkyl include methoxymethyl, ethoxymethyl, tert-butoxymethyl, 2-methoxyethyl, 2-ethoxyethyl, methoxypropyl, 2-methoxypropyl and methoxybutyl; examples of (1-4C)alkylS(O)p(1-4C)alkyl (where p is 0, 1 or 2) include methylsulfinylmethyl, ethylsulfinylmethyl, ethylsulfinylethyl, methylsulfinylpropyl, methylsulfinylbutyl, methylsulfonylmethyl, ethylsulfonylmethyl, ethylsulfonylethyl, methylsulfonylpropyl, methylsulfonylbutyl, methylthiomethyl, ethylthiomethyl, ethylthioethyl, methylthiopropyl, and methylthiobutyl; examples of amino(1-4C)alkyl include aminomethyl, aminoethyl, 2-aminopropyl, 3-aminopropyl, 1-aminoisopropyl and 4-aminobutyl; examples of (1-4C)alkylamino(1-4C)alkyl include (N-methyl)aminomethyl, (N-ethyl)aminomethyl, 1-((N-methyl)amino)ethyl, 2-((N-methyl)amino)ethyl, (N-ethyl)aminoethyl, (N-methyl)aminopropyl, and 4-((N-methyl)amino)butyl; examples of di(1-4C)alkylamino(1-4C)alkyl include dimethylaminomethyl, methyl(ethyl)aminomethyl, methyl(ethyl)aminoethyl, (N,N-diethyl)aminoethyl, (N,N-dimethyl)aminopropyl and (N,N-dimethyl)aminobutyl; examples of (1-4C)alkylcarbonyl include methylcarbonyl, ethylcarbonyl, isopropylcarbonyl and tertbutylcarbonyl; examples of (1-4C)alkylsulphonyl include methylsulphonyl, ethylsulphonyl, isopropylsulphonyl and tertbutylsulphonyl.

It is to be understood that, insofar as certain of the compounds of Formula (I) defined above may exist in optically active or racemic forms by virtue of one or more asymmetric carbon atoms, the invention includes in its definition any such optically active or racemic form which possesses the property of stimulating GLK directly or inhibiting the GLK/GLKRP interaction. The synthesis of optically active forms may be carried out by standard techniques of organic chemistry well known in the art, for example by synthesis from optically active starting materials or by resolution of a racemic form. It is also to be understood that certain compounds may exist in tautomeric forms and that the invention also relates to any and all tautomeric forms of the compounds of the invention which activate GLK.

It is also to be understood that certain compounds of the formula (I) and salts thereof can exist in solvated as well as unsolvated forms such as, for example, hydrated forms. It is to be understood that the invention encompasses all such solvated forms which activate GLK.

In one embodiment of the invention are provided compounds of formula (I), in an alternative embodiment are provided pharmaceutically-acceptable salts of compounds of formula (I), in a further alternative embodiment are provided in-vivo hydrolysable esters of compounds of formula (I), and in a further alternative embodiment are provided pharmaceutically-acceptable salts of in-vivo hydrolysable esters of compounds of formula (I).

Preferred values of each variable group are as follows. Such values may be used where appropriate with any of the values, definitions, claims, aspects or embodiments defined hereinbefore or hereinafter. In particular, each may be used as an individual limitation on the broadest definition of formula (I). Further, each of the following values may be used in combination with one or more of the other following values to limit the broadest definition of formula (I).

(1) R¹ is of sub-formula X:

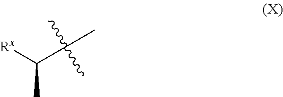

wherein R^{x} is selected from methyl, ethyl, trifluoromethyl, ethynyl, hydroxymethyl, hydroxyethyl, methoxymethyl, fluoromethoxymethyl, difluoromethoxymethyl and trifluoromethoxymethyl; preferably R^{x} is selected from methyl, ethyl, trifluoromethyl, ethynyl, hydroxymethyl, hydroxyethyl, methoxymethyl, fluoromethoxymethyl and difluoromethoxymethyl
(2) R¹ is of sub-formula Y:

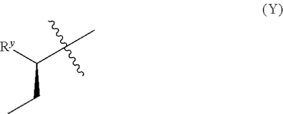

wherein R^{y} is selected from hydroxymethyl and methoxymethyl
(3) R¹ is 1-hydroxyprop-2-yl and the configuration is preferably (S), that is R¹—O— is:

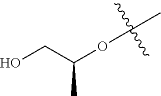

(4) R¹ is 1-methoxyprop-2-yl and the configuration is preferably (S), that is R¹—O— is:

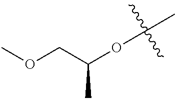

(5) R¹ is selected from isopropyl, 1,3-difluoroprop-2-yl, but-1-yn-3-yl, 1-hydroxyprop-2-yl, hydroxybut-3-yl and 1-methoxyprop-2-yl
(6) R¹ is 1,1,1-trifluoroprop-2-yl, 1-fluoromethoxyprop-2-yl, 1,1-difluoromethoxyprop-2-yl or 1-trifluoromethoxyprop-2-yl
(7) R¹ is 1-fluoromethoxyprop-2-yl, 1,1-difluoromethoxyprop-2-yl or 1-trifluoromethoxyprop-2-yl, particularly 1-fluoromethoxyprop-2-yl or 1,1-difluoromethoxyprop-2-yl
(8) R¹ is 1,1-difluoromethoxyprop-2-yl, particularly with the stereochemistry:

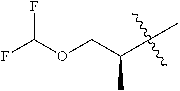

(9) R¹ is tetrahydrofuryl or tetrahydropyranyl
(10) R¹ is tetrahydrofuryl in the (S) configuration, that is:

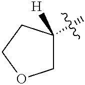

(11) R¹ is tetrahydrofuryl in the (R) configuration, that is:

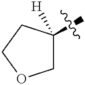

(12) R¹ is 4-tetrahydropyranyl:

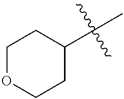

(13) R¹ is 2-hydroxy-but-3-yl and the configuration is preferably such that R¹—O— is:

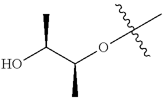

(14) R¹ is 1-hydroxybut-2-yl or 1-methoxybut-2-yl
(15) R¹ is selected from isopropyl, but-2-yl, 1,1,1-trifluoroprop-2-yl, 1,3-difluoroprop-2-yl, but-1-yn-3-yl, 1-hydroxyprop-2-yl, 2-hydroxybut-3-yl, tetrahydrofuryl, tetrahydropyranyl, 1-methoxyprop-2-yl, 1-fluoromethoxyprop-2-yl, 1,1-difluoromethoxyprop-2-yl and 1-trifluoromethoxyprop-2-yl
(16) R¹ is selected from 1-hydroxyprop-2-yl, 1,3-difluoroprop-2-yl, isopropyl and 1-methoxyprop-2-yl
(17) R¹ is selected from 2-hydroxyprop-1-yl, 2-methoxyprop-1-yl, 2-hydroxybut-1-yl and 2-methoxybut-1-yl
(18) R¹ is selected from isopropyl, 1,3-difluoroprop-2-yl, 1-hydroxyprop-2-yl, tetrahydrofuryl, 1-methoxyprop-2-yl and 1,1-difluoromethoxyprop-2-yl
(19) HET-1 is a 5-membered heteroaryl ring
(20) HET-1 is a 6-membered heteroaryl ring
(21) HET-1 is substituted with 1 or 2 substituents independently selected from R⁶
(22) HET-1 is substituted with 1 substituent selected from R⁶
(23) HET-1 is substituted with 1 substituent selected from R⁷
(24) HET-1 is unsubstituted
(25) HET-1 is selected from thiazolyl, isothiazolyl, thiadiazolyl, pyridyl, pyrazinyl, pyridazinyl, pyrazolyl, imidazolyl, pyrimidinyl, oxazolyl, isoxazolyl, oxadiazolyl, and triazolyl
(26) HET-1 is selected from thiazolyl, isothiazolyl, thiadiazolyl, pyrazolyl, imidazolyl, oxazolyl, isoxazolyl and oxadiazolyl
(27) HET-1 is selected from pyridyl, pyrazinyl, pyridazinyl and pyrimidinyl
(28) HET-1 is selected from thiazolyl, pyrazolyl and oxazolyl
(29) HET-1 is selected from thiadiazolyl and oxadiazolyl
(30) HET-1 is selected from 1,3,4-thiadiazolyl and 1,3,4-oxadiazolyl
(31) HET-1 is selected from 1,2,4-oxadiazolyl and 1,2,4-oxadiazolyl
(32) HET-1 is pyrazolyl, particularly N-methylpyrazolyl
(33) HET-1 is pyrazinyl
(34) HET-1 is selected from thiazolyl, pyrazolyl, thiadiazolyl and pyrazinyl
(35) HET-1 is pyrazolyl or pyrazinyl, particularly N-methylpyrazolyl or methylpyrazinyl
(36) HET-1 is pyrazolyl (optionally substituted with ethyl, isopropyl or 1 or 2 methyl), thiazolyl (optionally substituted with methyl), pyrazinyl (optionally substituted with methyl), pyridyl (optionally substituted by fluoro), isoxazolyl (optionally substituted with methyl) and thiadiazolyl (optionally substituted with methyl)
(37) HET-1 is pyrazolyl (optionally substituted with ethyl, isopropyl, difluoromethyl, or 1 or 2 methyl), thiazolyl (optionally substituted with methyl), pyrazinyl (optionally substituted with methyl), pyridyl (optionally substituted by fluoro), isoxazolyl (optionally substituted with methyl) and thiadiazolyl (optionally substituted with methyl)
(38) HET-1 is selected from pyrazinyl (optionally substituted with methyl), pyrazolyl (optionally substituted on carbon by methyl), methylthiadiazolyl (particularly 1,2,4-thiadiazol-5-yl, more particularly 3-methyl-1,2,4-thiadiazol-5-yl), thiazolyl (optionally substituted with methyl), pyridyl (optionally substituted by fluoro) and isoxazolyl
(39) R⁶ is selected from (1-4C)alkyl, halo, hydroxy(1-4C)alkyl and di(1-4C)alkylamino(1-4C)alkyl
(40) R⁶ is selected from methyl, ethyl, bromo, chloro, fluoro, hydroxymethyl, methoxymethyl,
aminomethyl, N-methylaminomethyl, dimethylaminomethyl
(41) R⁶ is selected from (1-4C)alkyl, halo, hydroxy(1-4C)alkyl, (1-4C)alkoxy(1-4C)alkyl, (1-4C)alkylS(O)p(1-4C)alkyl, amino(1-4C)alkyl, (1-4C)alkylamino(1-4C)alkyl, and di(1-4C)alkylamino(1-4C)alkyl
(42) R⁶ is selected from methyl, ethyl, chloro, fluoro, hydroxymethyl and methoxymethyl
(43) R⁶ is selected from methyl, ethyl, chloro and fluoro
(44) R⁶ is methyl
(45) R⁶ is selected from methyl, ethyl, chloro, fluoro, aminomethyl, N-methylaminomethyl, dimethylaminomethyl, hydroxymethyl and methoxymethyl
(46) R⁶ is selected from methyl, ethyl, aminomethyl, N-methylaminomethyl, dimethylaminomethyl, hydroxymethyl and methoxymethyl
(47) R⁶ is selected from methyl, ethyl, isopropyl and methoxymethyl
(48) when 2 substituents R⁶ are present, both are selected from methyl, ethyl, chloro and fluoro; preferably both are methyl
(49) R⁶ is selected from (1-4C)alkylS(O)p(1-4C)alkyl, (1-4C)alkylamino(1-4C)alkyl and di(1-4C)alkylamino(1-4C)alkyl
(50) R⁷ is selected from (1-4C)alkyl, hydroxy(1-4C)alkyl and di(1-4C)alkylamino(1-4C)alkyl
(51) R⁷ is selected from methyl, ethyl, hydroxymethyl, methoxymethyl, aminomethyl, N-methylaminomethyl, dimethylaminomethyl
(52) R⁷ is selected from (1-4C)alkyl, hydroxy(1-4C)alkyl, (1-4C)alkoxy(1-4C)alkyl, (1-4C)alkylS(O)p(1-4C)alkyl, amino(1-4C)alkyl, (1-4C)alkylamino(1-4C)alkyl, and di(1-4C)alkylamino(1-4C)alkyl
(53) R⁷ is selected from methyl, ethyl, aminomethyl, N-methylaminomethyl, and dimethylaminomethyl
(54) R⁷ is selected from methyl, ethyl, hydroxymethyl and methoxymethyl
(55) R⁷ is selected from methyl and ethyl
(56) R⁷ is methyl
(57) R⁷ is selected from methyl, ethyl, aminomethyl, N-methylaminomethyl, dimethylaminomethyl, hydroxymethyl and methoxymethyl
(58) R⁷ is selected from methyl, ethyl, isopropyl and methoxymethyl
(59) R² is (1-4C)alkyl, preferably methyl
(60) R² is selected from (1-4C)alkylcarbonyl, (1-4C)alkylsulphonyl, hydroxy(1-4C)alkyl and (1-4C)alkoxy(1-4C)alkyl
(61) R² is benzyl

### (62) R² is (3-6C)cycloalkyl

(63) R² is selected from (1-4C)alkyl, (3-6C)cycloalkyl and benzyl
(64) R³ is (1-4C)alkyl, preferably methyl
(65) R³ is hydroxy
(66) R³ is fluoro or chloro

### (67) R³ is (3-6C)cycloalkyl

### (68) R³ is (1-4C)alkoxy

(69) each R² and R³ is methyl
(70) R⁴ is a substituent on carbon and is fluoro or chloro;
(71) R⁴ is a substituent on nitrogen and is selected from (1-4C)alkyl, (3-6C)cycloalkyl and benzyl
(72) R⁴ is a substituent on nitrogen and is selected from (1-4C)alkylcarbonyl and (1-4C)alkylsulphonyl,
(73) R⁴ is a substituent on nitrogen and is selected from hydroxy(1-4C)alkyl and (1-4C)alkoxy(1-4C)alkyl
(74) Ring A is a 5-membered ring
(75) Ring A is a 6-membered ring
(76) Ring B is a 5-membered ring
(77) Ring B is a 6-membered ring
(78) Ring B is a 7-membered ring
(79) Ring B is unsubstituted
(80) Ring B is substituted on an available nitrogen atom by R²
(81) Ring B is substituted on each available nitrogen atom by a substituent R², wherein each R² is independently selected from (1-4C)alkyl and benzyl
(82) Ring B is substituted on an available carbon atom by R³
(83) Ring B is substituted on more than one available carbon atom by substituents independently selected from R³
(84) Ring B is substituted on one or more available carbon atom by methyl, and/or twice on one carbon atom by methyl
(85) Ring A is heteroaryl and Ring B is phenyl
(86) Ring A is heteroaryl and Ring B is heterocyclyl
(87) HET-2 is a 5,6 fused bicyclic system
(88) HET-2 is a 5,5 fused bicyclic system
(89) HET-2 is a 6,6 fused bicyclic system
(90) HET-2 is a 5,7 fused bicyclic system
(91) HET-2 is a 6,7 fused bicyclic system
(92) HET-2 is selected from structures A to F as hereinbefore defined, particularly wherein R² and R³ are both methyl and R⁴ is chloro or fluoro
(93) HET-2 is selected from structures G to P as hereinbefore defined

According to a further feature of the invention there is provided the following preferred groups of compounds of the invention:

In one aspect of the invention there is provided a compound of formula (I) or a salt thereof, wherein:

HET-1 is pyrazole, optionally substituted with methyl or ethyl;
R¹ is 1-hydroxyprop-2-yl, 1-methoxyprop-2-yl, 1,3-difluoroprop-2-yl or isopropyl;
R⁴ is fluoro or chloro;
HET-2 comprises Ring A and Ring B fused together as hereinbefore defined;
Ring A is pyridinyl or thiazolyl, optionally substituted with R⁴;
Ring B is phenyl or a 5 to 7 membered ring containing 1 to 3 heteroatoms independently selected from O, N and S, wherein a ring carbon or sulfur atom is optionally oxidised and a ring nitrogen atom is optionally substituted by a substituent selected from R² and a ring carbon atom is optionally substituted by 1 or 2 substituents independently selected from R³;
R² is selected from benzyl and (1-4C)alkyl; and
R³ is selected from (1-4C)alkyl, chloro and fluoro.

In another aspect of the invention there is provided a compound of formula (I) or a salt thereof, wherein:

HET-1 is pyrazole, optionally substituted with methyl or ethyl;
R¹ is 1-hydroxyprop-2-yl, 1-methoxyprop-2-yl, 1,3-difluoroprop-2-yl or isopropyl;
R⁴ is fluoro or chloro;
HET-2 comprises Ring A and Ring B fused together as hereinbefore defined;
Ring A is pyridinyl or thiazolyl, optionally substituted with R⁴;
Ring B is phenyl or a 5 to 7 membered ring containing 1 to 3 heteroatoms independently selected from O, N and S, wherein a ring carbon or sulfur atom is optionally oxidised and a ring nitrogen atom is optionally substituted by a substituent selected from R²;
R² is selected from benzyl and (1-4C)alkyl.

In another aspect of the invention there is provided a compound of formula (I) or a salt thereof, wherein:

HET-1 is pyrazole or pyrazinyl, optionally substituted with methyl or ethyl;
R¹ is 1-hydroxyprop-2-yl, 1-methoxyprop-2-yl, 1,3-difluoroprop-2-yl, tetrahydrofuryl, 1,1-difluoromethoxyprop-2-yl, or isopropyl;
R⁴ is fluoro or chloro;
HET-2 comprises Ring A and Ring B fused together as hereinbefore defined;
Ring A is pyridinyl or thiazolyl, optionally substituted with R⁴;
Ring B is phenyl or a 5 to 7 membered ring containing 1 to 3 heteroatoms independently selected from O, N and S, wherein a ring carbon or sulfur atom is optionally oxidised, a ring nitrogen atom is optionally substituted by a substituent selected from R² and a ring carbon atom is optionally substituted with 1 or 2 (1-4C)alkyl;
R² is selected from benzyl and (1-4C)alkyl.

In a further aspect is provided a compound of formula (I) or a salt thereof, wherein:

HET-1 is pyrazole, optionally substituted with methyl or ethyl;
R¹ is 1-hydroxyprop-2-yl, 1-methoxyprop-2-yl or isopropyl;
R⁴ is fluoro or chloro;
HET-2 comprises Ring A and Ring B fused together as hereinbefore defined;
Ring A is pyridinyl or pyrazinyl, optionally substituted with R⁴;
Ring B is phenyl or a 5 to 7 membered ring containing 1 to 3 heteroatoms independently selected from O, N and S, wherein a ring carbon or sulfur atom is optionally oxidised and a ring nitrogen atom is optionally substituted by a substituent selected from R² and a ring carbon atom is optionally substituted by 1 or 2 substituents independently selected from R³.
R² is selected from benzyl and (1-4C)alkyl; and
R³ is selected from (1-4C)alkyl, chloro and fluoro.

In a further aspect is provided a compound of formula (I) or a salt thereof, wherein:

### HET-1 is N-methylpyrazole;

R¹ is 1-hydroxyprop-2-yl;
R⁴ is fluoro or chloro;
HET-2 comprises Ring A and Ring B fused together as hereinbefore defined;
Ring A is pyridinyl or pyrazinyl, optionally substituted with R⁴;
Ring B is phenyl or a 5 to 7 membered ring containing 1 to 3 heteroatoms independently selected from O, N and S, wherein a ring carbon or sulfur atom is optionally oxidised and a ring nitrogen atom is optionally substituted by a substituent selected from R² and a ring carbon atom is optionally substituted by 1 or 2 substituents independently selected from R³;
R² is selected from benzyl, methyl and ethyl; and
R³ is selected from methyl and fluoro.

In a further aspect is provided a compound of formula (I) or a salt thereof, wherein:

HET-1 is pyrazole, optionally substituted with methyl or ethyl;
R¹ is 1-fluoromethoxyprop-2-yl, 1,1-difluoromethoxyprop-2-yl or 1-trifluoromethoxyprop-2-yl, particularly 1-fluoromethoxyprop-2-yl or 1,1-difluoromethoxyprop-2-yl;
R⁴ is fluoro or chloro;
HET-2 comprises Ring A and Ring B fused together as hereinbefore defined;
Ring A is pyridinyl or pyrazinyl, optionally substituted with R⁴;
Ring B is a phenyl or a 5 to 7 membered ring containing 1 to 3 heteroatoms independently selected from O, N and S, wherein a ring carbon or sulfur atom is optionally oxidised and a ring nitrogen atom is optionally substituted by a substituent selected from R² and a ring carbon atom is optionally substituted by 1 or 2 substituents independently selected from R³.
R² is selected from benzy and (1-4C)alkyl; and
R³ is selected from (1-4C)alkyl, chloro and fluoro.

In a further aspect is provided a compound of formula (I) or a salt thereof, wherein:

### HET-1 is N-methylpyrazole;

R¹ is 1-fluoromethoxyprop-2-yl, 1,1-difluoromethoxyprop-2-yl or 1-trifluoromethoxyprop-2-yl, particularly 1-fluoromethoxyprop-2-yl or 1,1-difluoromethoxyprop-2-yl;
R⁴ is fluoro or chloro;
HET-2 comprises Ring A and Ring B fused together as hereinbefore defined;
Ring A is pyridinyl or pyrazinyl, optionally substituted with R⁴;
Ring B is phenyl or a 5 to 7 membered ring containing 1 to 3 heteroatoms independently selected from O, N and S, wherein a ring carbon or sulfur atom is optionally oxidised and a ring nitrogen atom is optionally substituted by a substituent selected from R² and a ring carbon atom is optionally substituted by 1 or 2 substituents independently selected from R³;
R² is selected from benzyl, methyl and ethyl; and
R³ is selected from methyl and fluoro.

In a further aspect is provided a compound of formula (I) or a salt thereof, wherein:

### HET-1 is N-methylpyrazole;

R¹ is 1-hydroxyprop-2-yl;
HET-2 comprises Ring A and Ring B fused together as hereinbefore defined;
Ring A is thiazolyl;
Ring B is a phenyl or 5 to 7 membered ring containing 1 to 3 heteroatoms independently selected from O, N and S, wherein a ring carbon or sulfur atom is optionally oxidised and a ring nitrogen atom is optionally substituted by a substituent selected from R² and a ring carbon atom is optionally substituted by 1 or 2 substituents independently selected from R³;
R² is selected from benzyl, methyl and ethyl; and
R³ is selected from methyl and fluoro.

In another aspect of the invention there is provided a compound of formula (I) or a salt thereof, wherein:

HET-1 is pyrazole or pyrazinyl, optionally substituted with methyl or ethyl;
R¹ is 1-hydroxyprop-2-yl, 1-methoxyprop-2-yl, 1,3-difluoroprop-2-yl, tetrahydrofuryl, 1,1difluoromethoxyprop-2-yl, or isopropyl;
R⁴ is fluoro or chloro;
HET-2 is selected from formulae A to F as hereinbefore defined;
R² and R³ are both (1-4C)alkyl, particularly methyl.

In another aspect of the invention there is provided a compound of formula (I) or a salt thereof, wherein:

HET-1 is pyrazole or pyrazinyl, optionally substituted with methyl or ethyl;
R¹ is 1-hydroxyprop-2-yl, 1-methoxyprop-2-yl, 1,3-difluoroprop-2-yl, tetrahydrofuryl, 1,1difluoromethoxyprop-2-yl, or isopropyl;
HET-2 is selected from formulae G to P as hereinbefore defined.

Further preferred compounds of the invention are each of the Examples, each of which provides a further independent aspect of the invention. In further aspects, the present invention also comprises any two or more compounds of the Examples.

Particular compounds of the invention include:
3-(1,3-benzothiazol-2-yloxy)-5-{[(1S)-2-hydroxy-1-methylethyl]oxy}-N-(1-methyl-1H-pyrazol-3-yl)benzamide; and/or
3-{[2-fluoro-1-(fluoromethyl)ethyl]oxy}-5-[(4-methyl-5-oxo-2,3,4,5-tetrahydropyrido[3,2-f][1,4]oxazepin-8-yl)oxy]-N-(1-methyl-1H-pyrazol-3-yl)benzamide;
3-[(7-fluoro-4-methyl-5-oxo-2,3,4,5-tetrahydropyrido[3,2-f][1,4]oxazepin-8-yl)oxy]-5-[(1-methylethyl)oxy]-N-(1-methyl-1H-pyrazol-3-yl)benzamide;
3-[(7-fluoro-4-methyl-5-oxo-2,3,4,5-tetrahydropyrido[3,2-f][1,4]oxazepin-8-yl)oxy]-5-{[(1S)-1-methyl-2-(methyloxy)ethyl]oxy}-N-(1-methyl-1H-pyrazol-3-yl)benzamide;
3-[(1-methylethyl)oxy]-5-[(4-methyl-5-oxo-2,3,4,5-tetrahydropyrido[3,4-f][1,4]oxazepin-8-yl)oxy]-N-(1-methyl-1H-pyrazol-3-yl)benzamide;
3-{[(1S)-1-methyl-2-(methyloxy)ethyl]oxy}-N-(1-methyl-1H-pyrazol-3-yl)-5-[(4-oxo-5,6,7,8-tetrahydro-4H-[1,3]thiazolo[5,4-c]azepin-2-yl)oxy]benzamide; and
3-[(1-methylethyl)oxy]-N-(1-methyl-1H-pyrazol-3-yl)-5-[(4-oxo-5,6,7,8-tetrahydro-4H-[1,3]thiazolo[5,4-c]azepin-2-yl)oxy]benzamide; and/or
3-{[(1S)-1-methyl-2-(methyloxy)ethyl]oxy}-5-[(4-methyl-5-oxo-2,3,4,5-tetrahydropyrido[3,4-f][1,4]oxazepin-8-yl)oxy]-N-(1-methyl-1H-pyrazol-3-yl)benzamide;
3-[(2,3-dimethyl-4-oxo-3,4-dihydro-2H-pyrido[3,4-e][1,3]oxazin-7-yl)oxy]-5-{[(1S)-1-methyl-2-(methyloxy)ethyl]oxy}-N-(1-methyl-1H-pyrazol-3-yl)benzamide;
3-[(7,7-dimethyl-4-oxo-5,6,7,8-tetrahydro-4H-[1,3]thiazolo[5,4-c]azepin-2-yl)oxy]-5-{[(1S)-1-methyl-2-(methyloxy)ethyl]oxy}-N-(1-methyl-1H-pyrazol-3-yl)benzamide;
3-[(1-methylethyl)oxy]-N-(1-methyl-1H-pyrazol-3-yl)-5-[(5,7,7-trimethyl-4-oxo-5,6,7,8-tetrahydro-4H-[1,3]thiazolo[5,4-c]azepin-2-yl)oxy]benzamide;
3-{[(1S)-1-methyl-2-(methyloxy)ethyl]oxy}-N-(1-methyl-1H-pyrazol-3-yl)-5-[(5,7,7-trimethyl-4-oxo-5,6,7,8-tetrahydro-4H-[1,3]thiazolo[5,4-c]azepin-2-yl)oxy]benzamide;
3-[(1-methylethyl)oxy]-5-[(5-methyl-4-oxo-5,6,7,8-tetrahydro-4H-[1,3]thiazolo[5,4-c]azepin-2-yl)oxy]-N-(1-methyl-1H-pyrazol-3-yl)benzamide;
3-{[(1S)-1-methyl-2-(methyloxy)ethyl]oxy}-5-[(5-methyl-4-oxo-5,6,7,8-tetrahydro-4H-[1,3]thiazolo[5,4-c]azepin-2-yl)oxy]-N-(1-methyl-1H-pyrazol-3-yl)benzamide;
3-{[(1S)-1-methyl-2-(methyloxy)ethyl]oxy}-5-[(4-methyl-5-oxo-2,3,4,5-tetrahydropyrido[2,3-f][1,4]oxazepin-8-yl)oxy]-N-(5-methylpyrazin-2-yl)benzamide;
N-(1-methyl-1H-pyrazol-3-yl)-3-[(3S)-tetrahydrofuran-3-yloxy]-5-[(5,7,7-trimethyl-4-oxo-5,6,7,8-tetrahydro-4H-[1,3]thiazolo[5,4-c]azepin-2-yl)oxy]benzamide;
3-({(1S)-2-[(difluoromethyl)oxy]-1-methylethyl}oxy)-5-[(4-methyl-5-oxo-2,3,4,5-tetrahydropyrido[3,4-f][1,4]oxazepin-8-yl)oxy]-N-(1-methyl-1H-pyrazol-3-yl)benzamide;
   or a pharmaceutically-acceptable salt thereof.

The compounds of the invention may be administered in the form of a pro-drug. A pro-drug is a bioprecursor or pharmaceutically acceptable compound being degradable in the body to produce a compound of the invention (such as an ester or amide of a compound of the invention, particularly an in-vivo hydrolysable ester). Various forms of prodrugs are known in the art. For examples of such prodrug derivatives, see:
a) Design of Prodrugs, edited by H. Bundgaard, (Elsevier, 1985) and Methods in Enzymology, Vol. 42, p. 309-396, edited by K. Widder, et al. (Academic Press, 1985);
b) A Textbook of Drug Design and Development, edited by Krogsgaard-Larsen;
c) H. Bundgaard, Chapter 5 “Design and Application of Prodrugs”, by H. Bundgaard p. 113-191 (1991);
d) H. Bundgaard, Advanced Drug Delivery Reviews, 8, 1-38 (1992);
e) H. Bundgaard, et al., Journal of Pharmaceutical Sciences, 77, 285 (1988); and
f) N. Kakeya, et al., Chem Pharm Bull, 32, 692 (1984).
   The contents of the above cited documents are incorporated herein by reference.

Examples of pro-drugs are as follows. An in-vivo hydrolysable ester of a compound of the invention containing a carboxy or a hydroxy group is, for example, a pharmaceutically-acceptable ester which is hydrolysed in the human or animal body to produce the parent acid or alcohol. Suitable pharmaceutically-acceptable esters for carboxy include C₁ to C₆alkoxymethyl esters for example methoxymethyl, C₁ to C₆alkanoyloxymethyl esters for example pivaloyloxymethyl, phthalidyl esters, C₃ to C₈cycloalkoxycarbonyloxy C₁ to C₆alkyl esters for example 1-cyclohexylcarbonyloxyethyl; 1,3-dioxolen-2-onylmethyl esters, for example 5-methyl-1,3-dioxolen-2-onylmethyl; and C₁₋₆alkoxycarbonyloxyethyl esters.

An in-vivo hydrolysable ester of a compound of the invention containing a hydroxy group includes inorganic esters such as phosphate esters (including phosphoramidic cyclic esters) and α-acyloxyalkyl ethers and related compounds which as a result of the in-vivo hydrolysis of the ester breakdown to give the parent hydroxy group/s. Examples of α-acyloxyalkyl ethers include acetoxymethoxy and 2,2-dimethylpropionyloxy-methoxy. A selection of in-vivo hydrolysable ester forming groups for hydroxy include alkanoyl, benzoyl, phenylacetyl and substituted benzoyl and phenylacetyl, alkoxycarbonyl (to give alkyl carbonate esters), dialkylcarbamoyl and N-(dialkylaminoethyl)-N-alkylcarbamoyl (to give carbamates), dialkylaminoacetyl and carboxyacetyl.

A suitable pharmaceutically-acceptable salt of a compound of the invention is, for example, an acid-addition salt of a compound of the invention which is sufficiently basic, for example, an acid-addition salt with, for example, an inorganic or organic acid, for example hydrochloric, hydrobromic, sulphuric, phosphoric, trifluoroacetic, citric or maleic acid. In addition a suitable pharmaceutically-acceptable salt of a benzoxazinone derivative of the invention which is sufficiently acidic is an alkali metal salt, for example a sodium or potassium salt, an alkaline earth metal salt, for example a calcium or magnesium salt, an ammonium salt or a salt with an organic base which affords a physiologically-acceptable cation, for example a salt with methylamine, dimethylamine, trimethylamine, piperidine, morpholine or tris-(2-hydroxyethyl)amine.

A further feature of the invention is a pharmaceutical composition comprising a compound of Formula (I) as defined above, or a pharmaceutically-acceptable salt thereof, together with a pharmaceutically-acceptable diluent or carrier.

According to another aspect of the invention there is provided the a compound of Formula (I) as defined above or a pharmaceutically-acceptable salt thereof for use as a medicament.

According to another aspect of the invention there is provided a compound of Formula (I), or a pharmaceutically-acceptable salt thereof as defined above for use as a medicament for treatment of a disease mediated through GLK, in particular type 2 diabetes.

Further according to the invention there is provided the use of a compound of Formula (I) or a pharmaceutically-acceptable salt thereof in the preparation of a medicament for treatment of a disease mediated through GLK, in particular type 2 diabetes.

The compound is suitably formulated as a pharmaceutical composition for use in this way.

According to another aspect of the present invention there is provided a method of treating GLK mediated diseases, especially diabetes, by administering an effective amount of a compound of Formula (I) or a pharmaceutically-acceptable salt thereof, to a mammal in need of such treatment.

Specific diseases which may be treated by a compound or composition of the invention include: blood glucose lowering in Type 2 Diabetes Mellitus without a serious risk of hypoglycaemia (and potential to treat type 1), dyslipidemia, obesity, insulin resistance, metabolic syndrome X, impaired glucose tolerance.

As discussed above, thus the GLK/GLKRP system can be described as a potential “Diabesity” target (of benefit in both Diabetes and Obesity). Thus, according to another aspect of the invention there is provided the use of a compound of Formula (I) or a pharmaceutically-acceptable salt thereof, in the preparation of a medicament for use in the combined treatment or prevention, particularly treatment, of diabetes and obesity.

According to another aspect of the invention there is provided the use of a compound of Formula (I) or a pharmaceutically-acceptable salt thereof, in the preparation of a medicament for use in the treatment or prevention of obesity.

According to a further aspect of the invention there is provided a method for the combined treatment of obesity and diabetes by administering an effective amount of a compound of Formula (I) or a pharmaceutically-acceptable salt thereof, to a mammal in need of such treatment.

According to another aspect of the invention there is provided a compound of Formula (I) or a pharmaceutically-acceptable salt thereof as defined above for use as a medicament for treatment or prevention, particularly treatment of obesity.

According to a further aspect of the invention there is provided a method for the treatment of obesity by administering an effective amount of a compound of Formula (I) or a pharmaceutically-acceptable salt thereof, to a mammal in need of such treatment.

Compounds of the invention may be particularly suitable for use as pharmaceuticals because of advantageous physical and/or pharmacokinetic properties, and/or favourable toxicity profile.

The compositions of the invention may be in a form suitable for oral use (for example as tablets, lozenges, hard or soft capsules, aqueous or oily suspensions, emulsions, dispersible powders or granules, syrups or elixirs), for topical use (for example as creams, ointments, gels, or aqueous or oily solutions or suspensions), for administration by inhalation (for example as a finely divided powder or a liquid aerosol), for administration by insufflation (for example as a finely divided powder) or for parenteral administration (for example as a sterile aqueous or oily solution for intravenous, subcutaneous, intramuscular or intramuscular dosing or as a suppository for rectal dosing). Dosage forms suitable for oral use are preferred.

The compositions of the invention may be obtained by conventional procedures using conventional pharmaceutical excipients, well known in the art. Thus, compositions intended for oral use may contain, for example, one or more colouring, sweetening, flavouring and/or preservative agents.

Suitable pharmaceutically acceptable excipients for a tablet formulation include, for example, inert diluents such as lactose, sodium carbonate, calcium phosphate or calcium carbonate, granulating and disintegrating agents such as corn starch or algenic acid; binding agents such as starch; lubricating agents such as magnesium stearate, stearic acid or talc; preservative agents such as ethyl or propyl p-hydroxybenzoate, and anti-oxidants, such as ascorbic acid. Tablet formulations may be uncoated or coated either to modify their disintegration and the subsequent absorption of the active ingredient within the gastrointestinal tract, or to improve their stability and/or appearance, in either case, using conventional coating agents and procedures well known in the art.

Compositions for oral use may be in the form of hard gelatin capsules in which the active ingredient is mixed with an inert solid diluent, for example, calcium carbonate, calcium phosphate or kaolin, or as soft gelatin capsules in which the active ingredient is mixed with water or an oil such as peanut oil, liquid paraffin, or olive oil.

Aqueous suspensions generally contain the active ingredient in finely powdered form together with one or more suspending agents, such as sodium carboxymethylcellulose, methylcellulose, hydroxypropylmethylcellulose, sodium alginate, polyvinyl-pyrrolidone, gum tragacanth and gum acacia; dispersing or wetting agents such as lecithin or condensation products of an alkylene oxide with fatty acids (for example polyoxyethylene stearate), or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives (such as ethyl or propyl p-hydroxybenzoate, anti-oxidants (such as ascorbic acid), colouring agents, flavouring agents, and/or sweetening agents (such as sucrose, saccharine or aspartame).

Oily suspensions may be formulated by suspending the active ingredient in a vegetable oil (such as arachis oil, olive oil, sesame oil or coconut oil) or in a mineral oil (such as liquid paraffin). The oily suspensions may also contain a thickening agent such as beeswax, hard paraffin or cetyl alcohol. Sweetening agents such as those set out above, and flavouring agents may be added to provide a palatable oral preparation. These compositions may be preserved by the addition of an anti-oxidant such as ascorbic acid.

Dispersible powders and granules suitable for preparation of an aqueous suspension by the addition of water generally contain the active ingredient together with a dispersing or wetting agent, suspending agent and one or more preservatives. Suitable dispersing or wetting agents and suspending agents are exemplified by those already mentioned above. Additional excipients such as sweetening, flavouring and colouring agents, may also be present.

The pharmaceutical compositions of the invention may also be in the form of oil-in-water emulsions. The oily phase may be a vegetable oil, such as olive oil or arachis oil, or a mineral oil, such as for example liquid paraffin or a mixture of any of these. Suitable emulsifying agents may be, for example, naturally-occurring gums such as gum acacia or gum tragacanth, naturally-occurring phosphatides such as soya bean, lecithin, an esters or partial esters derived from fatty acids and hexitol anhydrides (for example sorbitan monooleate) and condensation products of the said partial esters with ethylene oxide such as polyoxyethylene sorbitan monooleate. The emulsions may also contain sweetening, flavouring and preservative agents.

Syrups and elixirs may be formulated with sweetening agents such as glycerol, propylene glycol, sorbitol, aspartame or sucrose, and may also contain a demulcent, preservative, flavouring and/or colouring agent.

The pharmaceutical compositions may also be in the form of a sterile injectable aqueous or oily suspension, which may be formulated according to known procedures using one or more of the appropriate dispersing or wetting agents and suspending agents, which have been mentioned above. A sterile injectable preparation may also be a sterile injectable solution or suspension in a non-toxic parenterally-acceptable diluent or solvent, for example a solution in 1,3-butanediol.

Compositions for administration by inhalation may be in the form of a conventional pressurised aerosol arranged to dispense the active ingredient either as an aerosol containing finely divided solid or liquid droplets. Conventional aerosol propellants such as volatile fluorinated hydrocarbons or hydrocarbons may be used and the aerosol device is conveniently arranged to dispense a metered quantity of active ingredient.

For further information on formulation the reader is referred to Chapter 25.2 in Volume 5 of Comprehensive Medicinal Chemistry (Corwin Hansch; Chairman of Editorial Board), Pergamon Press 1990.

The amount of active ingredient that is combined with one or more excipients to produce a single dosage form will necessarily vary depending upon the host treated and the particular route of administration. For example, a formulation intended for oral administration to humans will generally contain, for example, from 0.5 mg to 2 g of active agent compounded with an appropriate and convenient amount of excipients which may vary from about 5 to about 98 percent by weight of the total composition. Dosage unit forms will generally contain about 1 mg to about 500 mg of an active ingredient. For further information on Routes of Administration and Dosage Regimes the reader is referred to Chapter 25.3 in Volume 5 of Comprehensive Medicinal Chemistry (Corwin Hansch; Chairman of Editorial Board), Pergamon Press 1990.

The size of the dose for therapeutic or prophylactic purposes of a compound of the Formula (I) will naturally vary according to the nature and severity of the conditions, the age and sex of the animal or patient and the route of administration, according to well known principles of medicine.

In using a compound of the Formula (I) for therapeutic or prophylactic purposes it will generally be administered so that a daily dose in the range, for example, 0.5 mg to 75 mg per kg body weight is received, given if required in divided doses. In general lower doses will be administered when a parenteral route is employed. Thus, for example, for intravenous administration, a dose in the range, for example, 0.5 mg to 30 mg per kg body weight will generally be used. Similarly, for administration by inhalation, a dose in the range, for example, 0.5 mg to 25 mg per kg body weight will be used. Oral administration is however preferred.

The elevation of GLK activity described herein may be applied as a sole therapy or in combination with one or more other substances and/or treatments for the indication being treated. Such conjoint treatment may be achieved by way of the simultaneous, sequential or separate administration of the individual components of the treatment. Simultaneous treatment may be in a single tablet or in separate tablets. For example in the treatment of diabetes mellitus, chemotherapy may include the following main categories of treatment:

1) Insulin and insulin analogues;
2) Insulin secretagogues including sulphonylureas (for example glibenclamide, glipizide), prandial glucose regulators (for example repaglinide, nateglinide);
3) Agents that improve incretin action (for example dipeptidyl peptidase IV inhibitors, and GLP-1 agonists);
4) Insulin sensitising agents including PPARgamma agonists (for example pioglitazone and rosiglitazone), and agents with combined PPARalpha and gamma activity;
5) Agents that modulate hepatic glucose balance (for example metformin, fructose 1,6 bisphosphatase inhibitors, glycogen phosphorylase inhibitors, glycogen synthase kinase inhibitors);
6) Agents designed to reduce the absorption of glucose from the intestine (for example acarbose);
7) Agents that prevent the reabsorption of glucose by the kidney (SGLT inhibitors);
8) Agents designed to treat the complications of prolonged hyperglycaemia (for example aldose reductase inhibitors);
9) Anti-obesity agents (for example sibutramine and orlistat);
10) Anti-dyslipidaemia agents such as, HMG-CoA reductase inhibitors (e.g. statins); PPARα agonists (fibrates, e.g. gemfibrozil); bile acid sequestrants (cholestyramine); cholesterol absorption inhibitors (plant stanols, synthetic inhibitors); bile acid absorption inhibitors (IBATi) and nicotinic acid and analogues (niacin and slow release formulations);
11) Antihypertensive agents such as, β blockers (e.g. atenolol, inderal); ACE inhibitors (e.g. lisinopril); Calcium antagonists (e.g. nifedipine); Angiotensin receptor antagonists (e.g. candesartan), α antagonists and diuretic agents (e.g. furosemide, benzthiazide);
12) Haemostasis modulators such as, antithrombotics, activators of fibrinolysis and antiplatelet agents; thrombin antagonists; factor Xa inhibitors; factor VIIa inhibitors); antiplatelet agents (e.g. aspirin, clopidogrel); anticoagulants (heparin and Low molecular weight analogues, hirudin) and warfarin;
13) Agents which antagonise the actions of glucagon; and
14) Anti-inflammatory agents, such as non-steroidal anti-inflammatory drugs (e.g. aspirin) and steroidal anti-inflammatory agents (e.g. cortisone).

According to another aspect of the present invention there is provided individual compounds produced as end products in the Examples set out below and salts thereof.

A compound of the invention, or a salt thereof, may be prepared by any process known to be applicable to the preparation of such compounds or structurally related compounds. Functional groups may be protected and deprotected using conventional methods. For examples of protecting groups such as amino and carboxylic acid protecting groups (as well as means of formation and eventual deprotection), see T. W. Greene and P. G. M. Wuts, “Protective Groups in Organic Synthesis”, Second Edition, John Wiley & Sons, New York, 1991.

Processes for the synthesis of compounds of Formula (I) are provided as a further feature of the invention. Thus, according to a further aspect of the invention there is provided a process for the preparation of a compound of Formula (I), which comprises a process a) to e) (wherein the variables are as defined hereinbefore for compounds of Formula (I) unless otherwise defined):
(a) reaction of an acid of Formula (III) or activated derivative thereof with a compound of Formula (IV), wherein R¹ is as hereinbefore defined or a protected version thereof;

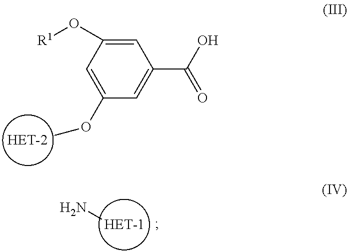

or
(b) reaction of a compound of Formula (V) with a compound of Formula (VI),

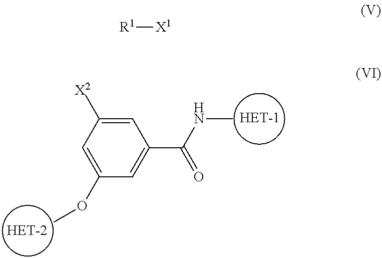

wherein X¹ is a leaving group and X² is a hydroxyl group or X¹ is a hydroxyl group and X² is a leaving group, and wherein R¹ is as hereinbefore defined or a protected version thereof;
process (b) could also be accomplished using the intermediate ester Formula (VII),
wherein P¹ is a protecting group as hereinafter described, followed by ester hydrolysis and amide formation by procedures described elsewhere and well known to those skilled in the art;

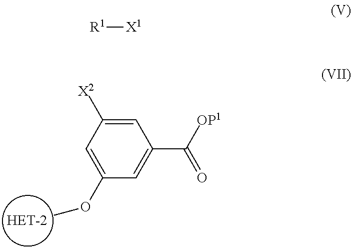

or
(c) reaction of a compound of Formula (VIII) with a compound of Formula (IX)

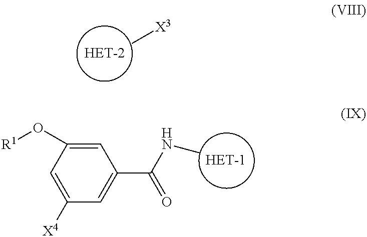

wherein X³ is a leaving group or an organometallic reagent and X⁴ is a hydroxyl group or
X³ is a hydroxyl group and X⁴ is a leaving group or an organometallic reagent, and wherein R¹ is as hereinbefore defined or a protected version thereof;
process (c) could also be accomplished using the intermediate ester Formula (X), followed by ester hydrolysis and amide formation by procedures described elsewhere and well known to those skilled in the art;

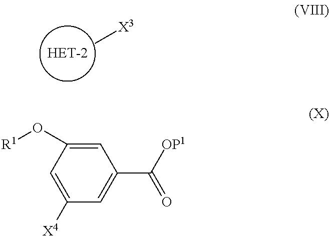

or
(d) reaction of a compound of Formula (XI) with a compound of Formula (XII),

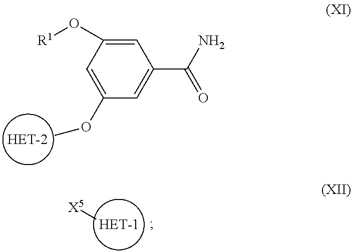

wherein X⁵ is a leaving group; and wherein R¹ is as hereinbefore defined or a protected version thereof; or
e) cyclisation of a compound of formula (XIII) to a compound of formula (I)

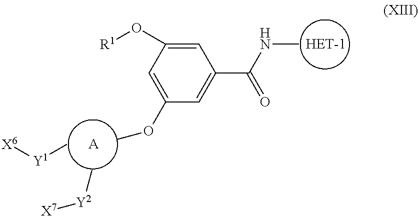

wherein Y¹ and Y² are 0-4 atom linkers attached to adjacent atoms in ring A, wherein each linker atom is independently selected from C, N, S or O (wherein any C or S can be optionally oxidised and any atom can be optionally substituted provided it is not quaternised and there are no S—S or O—O bonds), X⁶ can be any nucleophilic species and X⁷ a leaving group or vice versa, and wherein R¹ is as hereinbefore defined or a protected version thereof;
process (e) could also be accomplished using the intermediate ester Formula (XIV), followed by ester hydrolysis and amide formation by procedures described elsewhere and well known to those skilled in the art;

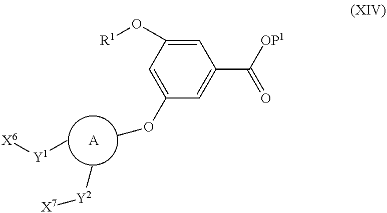

and thereafter, if necessary:
i) converting a compound of Formula (I) into another compound of Formula (I);
ii) removing any protecting groups; and/or
iii) forming a salt thereof.

Suitable leaving groups X¹ to X⁷ for processes b) to e) are any leaving group known in the art for these types of reactions, for example halo, alkoxy, trifluoromethanesulfonyloxy, methanesulfonyloxy, or p-toluenesulfonyloxy; or a group (such as a hydroxy group) that may be converted into a leaving group (such as an oxytriphenylphosphonium group) in situ.

Suitable values for R¹ containing a protected hydroxy group are any suitable protected hydroxy group known in the art, for example simple ethers such as a methyl ether, tert-butyl ether or silylethers such as —OSi[(1-4C)alkyl]₃ (wherein each (1-4C)alkyl group is independently selected from methyl, ethyl, propyl, isopropyl, and tertbutyl). Examples of such trialkylsilyl groups are trimethylsilyl, triethylsilyl, triisopropylsilyl and tert-butyldimethylsilyl. Further suitable silyl ethers are those containing phenyl and substituted phenyl groups, such as —Si(PhMe₂) and —Si(TolMe₂) (wherein Tol=methylbenzene). Further suitable values for hydroxy protecting groups are given hereinafter.

Compounds of Formulae (III) to (XV) are commercially available, or are known in the art, or may be made by processes known in the art, for example as shown in the accompanying Examples, or as described below. For further information on processes for making such compounds, we refer to our PCT publications WO 03/000267, WO 03/015774 and WO 03/000262 and references therein. In general it will be appreciated that any aryl-O or alkyl-O bond may be formed by nucleophilic substitution or metal catalysed processes, optionally in the presence of a suitable base.

Compounds of formulae (III), (IX), (X) and (XI) may be made by reaction of suitable precursors with compounds of formula (V) or derivatives thereof, depending on the nature of the R¹ group, for example, by nucleophilic displacement of a leaving group X¹ in a compound of formula (V). Compounds of formula (V) are generally commercially available or maybe made by simple functional group interconversions from commercially available compounds, or by literature methods. Further information is available in WO2004/076420, WO2005/054200, WO2005/054233, WO 2005/044801 and WO 2005/056530. Some illustrative examples using various R¹ groups are given in the Schemes below, and/or in the accompanying examples, and may generally be applied analogously to R¹ groups not shown below using methods known in the art, see for example Bull. Chem. Soc. Japan, 73 (2000), 471-484, International Patent application WO 2002/050003 and Bioorganic and Medicinal Chemistry Letters, (2001), 11, 407.

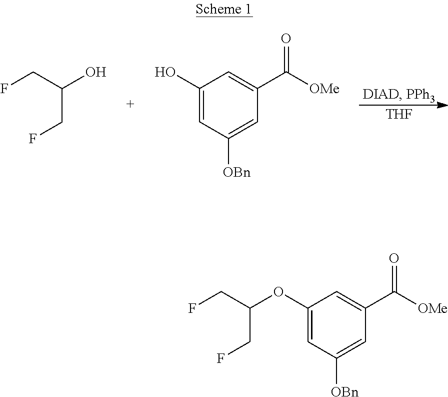

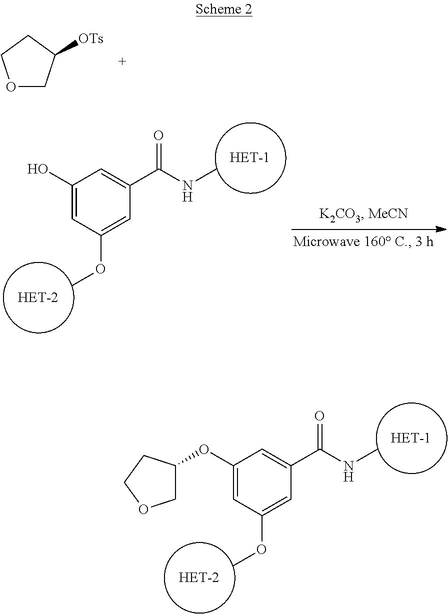

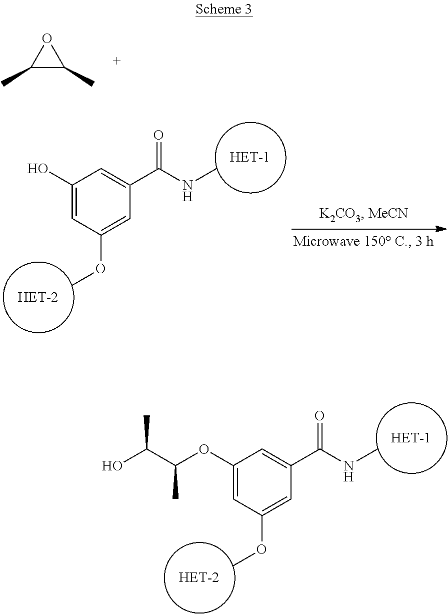

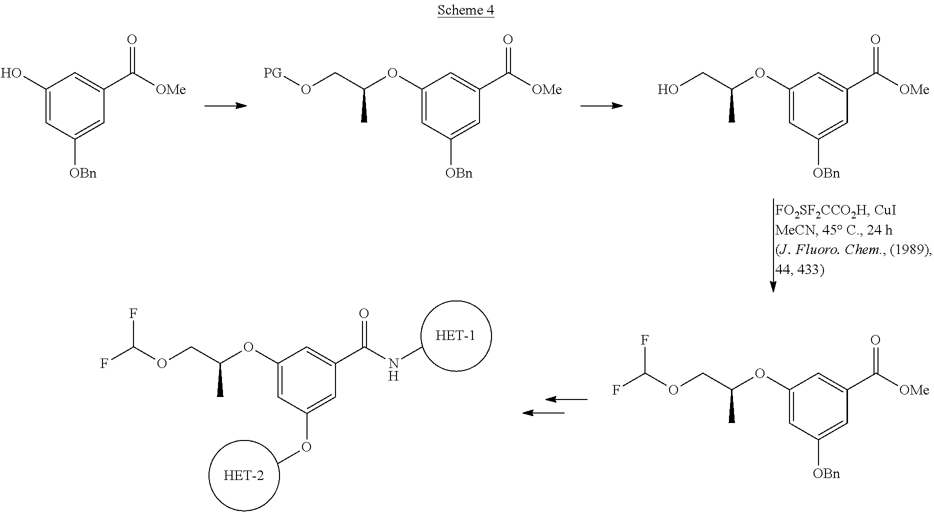

[PG is protecting group, Ts is p-toluenesulfonyl].

Examples of conversions of a compound of Formula (I) into another compound of Formula (I), well known to those skilled in the art, include functional group interconversions such as hydrolysis, hydrogenation, hydrogenolysis, oxidation or reduction, and/or further functionalisation by standard reactions such as amide or metal-catalysed coupling, or nucleophilic displacement reactions. Another example of a conversion of a compound of formula (I) into another compound of formula (I) also includes conversion of, for example, a hydroxymethyl group in R¹ (such as when R¹ is hydroxyprop-2-yl) into a difluoromethoxy group, using reactions such as those as illustrated in Scheme 4.

It will be understood that substituents R², R³, R⁴, R⁶ and/or R⁷ may be introduced into the molecule at any convenient point in the synthetic sequence or may be present in the starting materials. A precursor to one of these substituents may be present in the molecule during the process steps a) to e) above, and then be transformed into the desired substituent as a final step to form the compound of formula (I); followed where necessary by

i) converting a compound of Formula (I) into another compound of Formula (I);
ii) removing any protecting groups; and/or
iii) forming a salt thereof.

Specific reaction conditions for the above reactions are as follows, wherein when P¹ is a protecting group P¹ is preferably (1-4C)alkyl, for example methyl or ethyl:

Process a)—coupling reactions of amino groups with carboxylic acids to form an amide are well known in the art. For example,
(i) using an appropriate coupling reaction, such as a carbodiimide coupling reaction performed with EDAC (1-(3-dimethylaminopropyl)-3-ethylcarbodiimide hydrochloride) in the presence of dimethylaminopyridine (DMAP) in a suitable solvent such as dichloromethane (DCM), chloroform or dimethylformamide (DMF) at room temperature; or
(ii) reaction in which the carboxylic group is activated to an acid chloride by reaction with oxalyl chloride in the presence of a suitable solvent such as DCM. The acid chloride can then be reacted with a compound of Formula (IV) in the presence of a base, such as triethylamine or pyridine, in a suitable solvent such as chloroform or DCM at a temperature between 0° C. and 80° C.
Process b)—compounds of Formula (V) and (VI) can be reacted together in a suitable solvent, such as DMF or tetrahydrofuran (THF), with a base such as sodium hydride or potassium tert-butoxide, at a temperature in the range 0 to 200° C., optionally using microwave heating or metal catalysis such as palladium(II)acetate, palladium on carbon, copper(II)acetate or copper(I)iodide; alternatively, compounds of Formula (V) and (VI) can be reacted together in a suitable solvent, such as THF or DCM, with a suitable phosphine such as triphenylphosphine, and azodicarboxylate such as diethylazodicarboxylate; process b) could also be carried out using a precursor to the ester of formula (VII) such as an aryl-nitrile or trifluoromethyl derivative, followed by conversion to a carboxylic acid and amide formation as previously described;
Process c)—compounds of Formula (VIII) and (IX) can be reacted together in a suitable solvent, such as DMF or THF, with a base such as sodium hydride or potassium tert-butoxide, at a temperature in the range 0 to 200° C., optionally using microwave heating or metal catalysis such as palladium(II)acetate, palladium on carbon, copper(II)acetate or copper(I)iodide; process c) could also be carried out using a precursor to the ester of formula (X) such as an aryl-nitrile or trifluoromethyl derivative, followed by conversion to a carboxylic acid and amide formation as previously described;
compounds of the formula (VIII) are commercially available or can be prepared from commercially available materials by processes well known to those skilled in the art, for example functional group interconversions (such as hydrolysis, hydrogenation, hydrogenolysis, oxidation or reduction), and/or further functionalisation and/or cyclisation by standard reactions (such as amide or sulphonamide or metal-catalysed coupling, or nucleophilic displacement or electrophilic substitution reactions);
Process d)—reaction of a compound of Formula (XI) with a compound of Formula (XII) can be performed in a polar solvent, such as DMF or a non-polar solvent such as THF with a strong base, such as sodium hydride or potassium tert-butoxide at a temperature between 0 and 200° C., optionally using microwave heating or metal catalysis, such as palladium(II)acetate, palladium on carbon, copper(II)acetate or copper(I)iodide;
Process e)—cyclisation of a compound of formula (XIII) to a compound of formula (I) are well known in the art; for example,
i) a coupling reaction of amino groups with carboxylic acids using coupling reagents or acid chlorides (see process a) to form amide bonds;
ii) a coupling reaction of an amino group with a sulphonyl chloride in the presence of a suitable base, such as pyridine or triethylamine, in a suitable solvent such as DCM, toluene or pyridine at a temperature between 0° C. and 80° C., to form a sulphonamide group;
iii) reaction with a suitable solvent, such as DMF or tetrahydrofuran (THF), with a base such as sodium hydride or potassium tert-butoxide, at a temperature in the range 0 to 200° C., optionally using microwave heating or metal catalysis such as palladium(II)acetate, palladium on carbon, copper(II)acetate or copper(I)iodide; alternatively, reaction in a suitable solvent, such as THF or DCM, with a suitable phosphine such as triphenylphosphine, and azodicarboxylate such as diethylazodicarboxylate;
iv) electrophilic substitution reactions (such as Friedel Crafts reactions, for compounds of Formula (XIII) where either Y¹ is a direct bond and X⁶═H or Y² is a direct bond and X⁷ is H);
compounds of the Formula (XIII) may be made from compounds of Formula (XV), wherein the two R groups are attached to adjacent atoms in Ring A and each R group is independently a simple substituent (such as halo or cyano) or hydrogen, by processes well known to those skilled in the art such as functional group interconversions (for example hydrolysis, hydrogenation, hydrogenolysis, oxidation or reduction), and/or further functionalisation by standard reactions (such as amide or sulphonamide or metal-catalysed coupling, or nucleophilic displacement or electrophilic substitution reactions); compounds of formula (XV) may be made from commercially available materials by processes such as those described herein in processes a) to e).

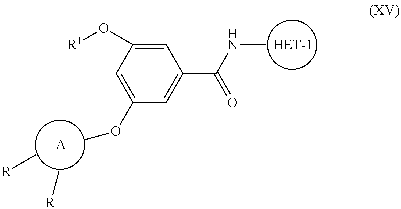

Certain intermediates of formula (III), (VI), (VII), (IX), (XI) and/or (XIII) and/or are believed to be novel and comprise an independent aspect of the invention.

Certain intermediates of formula (III), (IX) and/or (XI) wherein R¹ is as defined herein for a compound of formula (I) are believed to be novel and comprise an independent aspect of the invention.

During the preparation process, it may be advantageous to use a protecting group for a functional group within the molecule. Protecting groups may be removed by any convenient method as described in the literature or known to the skilled chemist as appropriate for the removal of the protecting group in question, such methods being chosen so as to effect removal of the protecting group with minimum disturbance of groups elsewhere in the molecule.

Specific examples of protecting groups are given below for the sake of convenience, in which “lower” signifies that the group to which it is applied preferably has 1-4 carbon atoms. It will be understood that these examples are not exhaustive. Where specific examples of methods for the removal of protecting groups are given below these are similarly not exhaustive. The use of protecting groups and methods of deprotection not specifically mentioned is of course within the scope of the invention.

A carboxy protecting group may be the residue of an ester-forming aliphatic or araliphatic alcohol or of an ester-forming silanol (the said alcohol or silanol preferably containing 1-20 carbon atoms). Examples of carboxy protecting groups include straight or branched chain (1-12C)alkyl groups (e.g. isopropyl, t-butyl); lower alkoxy lower alkyl groups (e.g. methoxymethyl, ethoxymethyl, isobutoxymethyl); lower aliphatic acyloxy lower alkyl groups, (e.g. acetoxymethyl, propionyloxymethyl, butyryloxymethyl, pivaloyloxymethyl); lower alkoxycarbonyloxy lower alkyl groups (e.g. 1-methoxycarbonyloxyethyl, 1-ethoxycarbonyloxyethyl); aryl lower alkyl groups (e.g. p-methoxybenzyl, o-nitrobenzyl, p-nitrobenzyl, benzhydryl and phthalidyl); tri(lower alkyl)silyl groups (e.g. trimethylsilyl and t-butyldimethylsilyl); tri(lower alkyl)silyl lower alkyl groups (e.g. trimethylsilylethyl); and (2-6C)alkenyl groups (e.g. allyl and vinylethyl).

Methods particularly appropriate for the removal of carboxyl protecting groups include for example acid-, metal- or enzymically-catalysed hydrolysis. Hydrogenation may also be used.

Examples of hydroxy protecting groups include methyl, t-butyl, lower alkenyl groups (e.g. allyl); lower alkanoyl groups (e.g. acetyl); lower alkoxycarbonyl groups (e.g. t-butoxycarbonyl); lower alkenyloxycarbonyl groups (e.g. allyloxycarbonyl); aryl lower alkoxycarbonyl groups (e.g. benzoyloxycarbonyl, p-methoxybenzyloxycarbonyl, o-nitrobenzyloxycarbonyl, p-nitrobenzyloxycarbonyl); tri lower alkyl/arylsilyl groups (e.g. trimethylsilyl, t-butyldimethylsilyl, t-butyldiphenylsilyl); tetrahydropyran-2-yl; aryl lower alkyl groups (e.g. benzyl) groups; and triaryl lower alkyl groups (e.g. triphenylmethyl). Examples of amino protecting groups include formyl, aralkyl groups (e.g. benzyl and substituted benzyl, e.g. p-methoxybenzyl, nitrobenzyl and 2,4-dimethoxybenzyl, and triphenylmethyl); di-p-anisylmethyl and furylmethyl groups; lower alkoxycarbonyl (e.g. t-butoxycarbonyl); lower alkenyloxycarbonyl (e.g. allyloxycarbonyl); aryl lower alkoxycarbonyl groups (e.g. benzyloxycarbonyl, p-methoxybenzyloxycarbonyl, o-nitrobenzyloxycarbonyl, p-nitrobenzyloxycarbonyl; trialkylsilyl (e.g. trimethylsilyl and t-butyldimethylsilyl); alkylidene (e.g. methylidene); benzylidene and substituted benzylidene groups.

Methods appropriate for removal of hydroxy and amino protecting groups include, for example, hydrogenation, nucleophilic displacement, acid-, base, metal- or enzymically-catalysed hydrolysis, catalytic hydrogenolysis or photolytically for groups such as o-nitrobenzyloxycarbonyl, or with fluoride ions for silyl groups. For example, methylether protecting groups for hydroxy groups may be removed by trimethylsilyliodide. A tert-butyl ether protecting group for a hydroxy group may be removed by hydrolysis, for example by use of hydrochloric acid in methanol.

Examples of protecting groups for amide groups include aralkoxymethyl (e.g. benzyloxymethyl and substituted benzyloxymethyl); alkoxymethyl (e.g. methoxymethyl and trimethylsilylethoxymethyl); tri alkyl/arylsilyl (e.g. trimethylsilyl, t-butyldimethylsily, t-butyldiphenylsilyl); tri alkyl/arylsilyloxymethyl (e.g. t-butyldimethylsilyloxymethyl, t-butyldiphenylsilyloxymethyl); 4-alkoxyphenyl (e.g. 4-methoxyphenyl); 2,4-di(alkoxy)phenyl (e.g. 2,4-dimethoxyphenyl); 4-alkoxybenzyl (e.g. 4-methoxybenzyl); 2,4-di(alkoxy)benzyl (e.g. 2,4-di(methoxy)benzyl); and alk-1-enyl (e.g. allyl, but-1-enyl and substituted vinyl e.g. 2-phenylvinyl).

Aralkoxymethyl, groups may be introduced onto the amide group by reacting the latter group with the appropriate aralkoxymethyl chloride, and removed by catalytic hydrogenation. Alkoxymethyl, tri alkyl/arylsilyl and tri alkyl/silyloxymethyl groups may be introduced by reacting the amide with the appropriate chloride and removing with acid; or in the case of the silyl containing groups, fluoride ions. The alkoxyphenyl and alkoxybenzyl groups are conveniently introduced by arylation or alkylation with an appropriate halide and removed by oxidation with eerie ammonium nitrate. Finally alk-1-enyl groups may be introduced by reacting the amide with the appropriate aldehyde and removed with acid.

In the above other pharmaceutical composition, process, method, use and medicament manufacture features, the alternative and preferred aspects and embodiments of the compounds of the invention described herein also apply.

The following examples are for illustration purposes and are not intended to limit the scope of this application. Each exemplified compound represents a particular and independent aspect of the invention. In the following non-limiting Examples, unless otherwise stated:
(i) evaporations were carried out by rotary evaporation in vacuo and work-up procedures were carried out after removal of residual solids such as drying agents by filtration;
(ii) operations were carried out at room temperature, that is in the range 18-25° C. and under an atmosphere of an inert gas such as argon or nitrogen;
(iii) yields are given for illustration only and are not necessarily the maximum attainable;
(iv) the structures of the end-products of the Formula (I) were confirmed by nuclear (generally proton) magnetic resonance (NMR) with a field strength (for proton) of 300 MHz (generally using a Varian Gemini 2000) or 400 MHz (generally using a Bruker Avance DPX400), unless otherwise stated, and mass spectral techniques; proton magnetic resonance chemical shift values were measured on the delta scale and peak multiplicities are shown as follows: s, singlet; d, doublet; t, triplet; m, multiplet; br, broad; q, quartet, quin, quintet;
(v) intermediates were not generally fully characterised and purity was assessed by thin layer chromatography (TLC), high-performance liquid chromatography (HPLC), infra-red (IR) or NMR analysis;
(vi) Purification by chromatography generally refers to flash column chromatography, on silica unless otherwise stated. Column chromatography was generally carried out using prepacked silica cartridges (from 4 g up to 400 g) such as Redisep™ (available, for example, from Presearch Ltd, Hitchin, Herts, UK) or Biotage (Biotage UK Ltd, Hertford, Herts, UK), eluted using a pump and fraction collector system. Purification by Solid Phase Extraction (SPE) methods generally refers to the use of chromatography cartridges packed with SPE materials such as ISOLUTE® SCX-2 columns (available, for example, From International Sorbent Technology Ltd, Dryffryn Business Park, Hengoed, Mid Glamorgan, UK);
(vii) Mass spectra (MS) data was generated on an LCMS system where the HPLC component comprised generally either a Agilent 1100 or Waters Alliance HT (2790 & 2795) equipment and was run on a Phemonenex Gemini C18 5 μm, 50×2 mm column (or similar) eluting with either acidic eluent (for example, using a gradient between 0-95% water/acetonitrile with 5% of a 1% formic acid in 50:50 water:acetonitrile (v/v) mixture; or using an equivalent solvent system with methanol instead of acetonitrile), or basic eluent (for example, using a gradient between 0-95% water/acetonitrile with 5% of a 0.1% 880 Ammonia in acetonitrile mixture); and the MS component comprised generally a Waters ZQ spectrometer. Chromatograms for Electrospray (ESI) positive and negative Base Peak Intensity, and UV Total Absorption Chromatogram from 220-300 nm, are generated and values for m/z are given; generally, only ions which indicate the parent mass are reported and unless otherwise stated the value quoted is (M−H)⁻;
(viii) Suitable microwave reactors include “Smith Creator”, “CEM Explorer”, “Biotage Initiator sixty” and “Biotage Initiator eight”.

### Abbreviations

DCM dichloromethane;
DEAD diethylazodicarboxylate;
DIAD diisopropylazodicarboxylate;
DIPEA N,N-Diisopropylethylamine;
DMA dimethylacetamide
DMSO dimethyl sulphoxide;
DMF dimethylformamide;
EDAC 1-(3-dimethylaminopropyl)-3-ethylcarbodiimide hydrochloride;
HATU O-(7-Azabenzotriazol-1-yl)-N,N,N′,N′-tetramethyluronium hexofluorophosphate
HPLC high pressure liquid chromatography
HPMC Hydroxypropylmethylcellulose;
LCMS liquid chromatography/mass spectroscopy;
NMP N-methyl-2-pyrrolidone;
NMR nuclear magnetic resonance spectroscopy;
RT room temperature;
THF tetrahydrofuran;
TFA trifluoroacetic acid;
CDCl₃ deuterochloroform;
MgSO₄ magnesium sulfate.
   All compound names were derived using ACD NAME computer package.

### EXAMPLE 1

### 3-(1,3-Benzothiazol-2-yloxy)-5-{[(1S)-2-hydroxy-1-methylethyl]oxy}-N-(1-methyl-1H-pyrazol-3-yl)benzamide

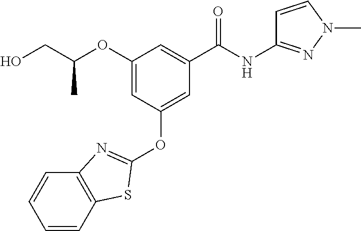

10% Hydrochloric acid (1.5 mL) was added to a solution of 3-(1,3-benzothiazol-2-yloxy)-5-[((1S)-2-{[(1,1-dimethylethyl)(dimethyl)silyl]oxy}-1-methylethyl)oxy]-N-(1-methyl-1H-pyrazol-3-yl)benzamide (410 mg, 0.76 mmol) in methanol (15 mL). The reaction was stirred at RT for 1 hour, saturated sodium bicarbonate solution added and the methanol evaporated. The aqueous residue was adjusted to pH 2 and extracted with ethyl acetate. The extracts were combined, washed with brine, dried (MgSO₄), filtered and evaporated in vacuo to give the crude product which was chromatographed on silica, eluting with 75% ethyl acetate in isohexane, to give the desired product (213 mg). ¹H NMR δ (CDCl₃): 1.3 (d, 3H), 2.5 (br, 1H), 3.65 (d, 2H), 3.7 (s, 3H), 4.5 (m, 1H), 6.8 (s, 1H), 7.1 (s, 1H), 7.25 (s, 1H), 7.3 (d, 1H), 7.4 (d, 2H), 7.45 (s, 1H), 7.7 (m, 2H), 9.1 (s, 1H). m/z 425 (M+H)⁺.

The preparation of 3-(1,3-benzothiazol-2-yloxy)-5-[((1S)-2-{[(1,1-dimethylethyl)(dimethyl)silyl]oxy}-1-methylethyl)oxy]-N-(1-methyl-1H-pyrazol-3-yl)benzamide is described below:

### 3-(1,3-Benzothiazol-2-yloxy)-5-[((1S)-2-{[(1,1-dimethylethyl)(dimethyl)silyl]oxy}-1-methylethyl)oxy]-N-(1-methyl-1H-pyrazol-3-yl)benzamide

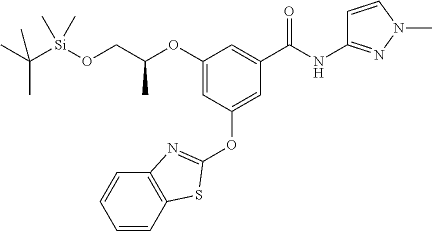

Cesium carbonate (652 mg, 2.0 mmol) was added to a solution of 3-[((1S)-2-{[(1,1-dimethylethyl)(dimethyl)silyl]oxy}-1-methylethyl)oxy]-5-hydroxy-N-(1-methyl-1H-pyrazol-3-yl)benzamide (404 mg, 1.0 mmol) and 2-bromobenzothiazole (216 mg, 1.0 mmol) in acetonitrile (20 mL) and the stirred mixture heated at 150° C. in a Biotage Initiator Microwave apparatus for 1 hour. The mixture was cooled to RT and pressure, poured onto water (300 mL), extracted with ethyl acetate (3×100 mL), the combined organic layers washed with brine, dried (MgSO₄) and evaporated to a residue which was chromatographed on silica, eluting with 30% ethyl acetate in isohexane, to give the desired product (422 mg). ¹H NMR δ (CDCl₃): 0.0 (d, 6H), 0.8 (s, 9H), 1.3 (d, 3H), 3.65 (d, 2H), 3.7 (s, 3H), 4.5 (m, 1H), 6.8 (s, 1H), 7.1 (s, 1H), 7.25 (m, 2H), 7.4 (m, 3H), 7.7 (m, 2H) and 8.7 (s, 1H). m/z 539 (M+H)⁺.

### 3-[((1S)-2-{[(1,1-Dimethylethyl)(dimethyl)silyl]oxy}-1-methylethyl)oxy]-5-hydroxy-N-(1-methyl-1H-pyrazol-3-yl)benzamide

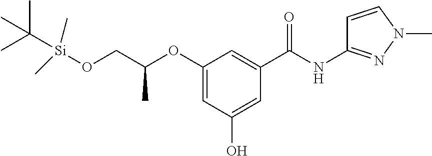

3-[((1S)-2-{[(1,1-dimethylethyl)(dimethyl)silyl]oxy}-1-methylethyl)oxy]-N-(1-methyl-1H-pyrazol-3-yl)-5-[(phenylmethyl)oxy]benzamide (1.8 g, 3.64 mmol) was dissolved in methanol (50 mL) and the flask evacuated and purged with nitrogen (3 times). 10% Palladium on carbon (0.2 g) was added and the flask further evacuated and finally purged with hydrogen gas. The reaction mixture was stirred at ambient temperature for 16 hours until completion. The reaction mixture was evacuated and purged with nitrogen (3 times). The catalyst was filtered off, and the filtrate concentrated in vacuo to give the desired compound (1.45 g).

¹H NMR δ (d₆-DMSO): 0.02 (d, 6H), 0.83 (s, 9H), 1.18 (d, 3H), 3.66 (m, 2H), 3.72 (s, 3H), 4.51 (m, 1H), 6.42 (m, 1H), 6.52 (m, 1H), 6.90 (s, 1H), 7.02 (s, 1H), 7.55 (m, 1H), 9.58 (br s, 1H), 10.59 (br s, 1H). m/z 406 (M+H)⁺

### 3-[((1S)-2-{[(1,1-Dimethylethyl)(dimethyl)silyl]oxy}-1-methylethyl)oxy]-N-(1-methyl-1H-pyrazol-3-yl)-5-[(phenylmethyl)oxy]benzamide

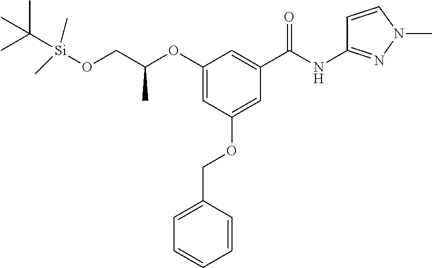

DIPEA (4.06 g, 23.4 mmol) was added to a suspension of 3-[((1S)-2-{[(1,1-dimethylethyl)(dimethyl)silyl]oxy}-1-methylethyl)oxy]-5-[(phenylmethyl)oxy]benzoic acid (2.43 g, 5.84 mmol), 3-amino-1-methylpyrazole (0.85 g, 8.76 mmol) and HATU (4.66 g, 12.3 mmol) in DMF (50 mL) and stirred at ambient temperature for 16 hours. The resultant mixture was partially reduced in vacuo, poured onto water (100 mL) and extracted with diethyl ether (2×50 mL). The extracts were washed with water and brine then dried (MgSO₄), filtered and reduced to an opaque gum which partially crystallized. The crude product was purified by column chromatography, eluting with 0-100% ethyl acetate in isohexane, to give the title compound as a colourless oil (1.87 g).

¹H NMR δ (d₆-DMSO): 0.02 (d, 6H), 0.84 (s, 9H), 1.21 (d, 3H), 3.68 (d, 2H), 3.76 (s, 3H), 4.58 (m, 1H), 5.13 (s, 2H), 6.56 (m, 1H), 6.70 (m, 1H), 7.18 (s, 1H), 7.24 (s, 1H), 7.29-7.46 (m, 5H), 7.57 (m, 1H), 10.74 (br s, 1H). m/z 496 (M+H)⁺

### 3-[((1S)-2-{[1,1-Dimethylethyl)(dimethyl)silyl]oxy}-1-methylethyl)oxy]-5-[(phenylmethyl)oxy]benzoic acid

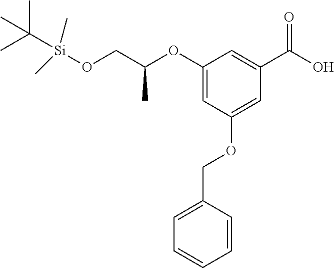

Methyl 3-[((1S)-2-{[(1,1-dimethylethyl)(dimethyl)silyl]oxy}-1-methylethyl)oxy]-5-[(phenylmethyl)oxy]benzoate (3.0 g, 6.98 mmol) was dissolved in THF (50 mL) and water (10 mL) and lithium hydroxide monohydrate (586 mg, 13.95 mmol) added. The resultant mixture was heated with stirring at 45° C. for 2 hours, then at ambient temperature for 16 hours, and at 45° C. for a further 4 hours. Water (40 mL) was added and the solvent removed in vacuo. The resultant solution was acidified carefully with 1M citric acid (2 equivalents), washed with water and brine then dried (MgSO₄), filtered and evaporated in vacuo to give the title compound as a colourless gum (2.58 g). ¹H NMR δ (d₆-DMSO): 0.02 (d, 6H), 0.84 (s, 9H), 1.17 (d, 3H), 3.66 (m, 2H), 4.43 (m, 1H), 5.05 (s, 2H), 6.56 (br s, 1H), 7.10 (br s, 1H), 7.17 (br s, 1H), 7.25-7.44 (m, 5H), 7.60 (br s, 1H).

### Methyl 3-[((1S)-2-{[(1,1-dimethylethyl)(dimethyl)silyl]oxy}-1-methylethyl)oxy]-5-[(phenylmethyl)oxy]benzoate

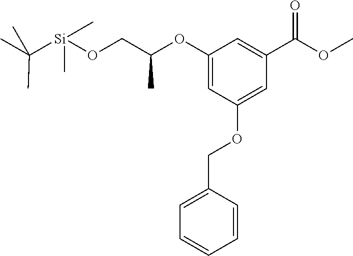

(2R)-1-{[(1,1-Dimethylethyl)(dimethyl)silyl]oxy}propan-2-ol (3.31 g, 17.4 mmol) was added to a solution of methyl 3-hydroxy-5-{[phenylmethyl]oxy}benzoate (3.00 g, 11.6 mmol) in THF (50 mL) at 0° C. followed by addition of triphenylphosphine (4.57 g, 17.4 mmol) then DIAD (3.43 mL, 17.4 mmol) and the reaction was warmed to RT and stirred for 16 h. The reaction was quenched with water (100 mL) and diethyl ether (400 mL) and the organic layer was separated then dried (MgSO₄) and evaporated. Purification by column chromatography, eluting with 1:15 to 1:5 ethyl acetate:hexane, afforded the title compound as a colourless oil (4.00 g, 80%).

¹H NMR δ (CDCl₃): 0.03 (s, 3H), 0.05 (s, 3H), 0.89 (s, 9H), 1.29 (d, 3H), 3.63 (dd, 1H), 3.78 (dd, 1H), 3.92 (s, 3H), 4.44 (m, 1H), 5.08 (s, 2H), 6.77 (m, 1H), 7.40 (m, 7H)

### Methyl 3-hydroxy-5-{[phenylmethyl]oxy}benzoate

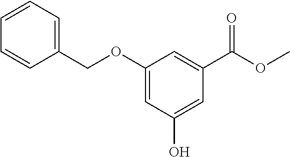

To a stirred solution of methyl 3,5-dihydroxybenzoate (5.95 mol) in DMF (6 L) was added potassium carbonate (9 mol), and the suspension stirred at ambient temperature under argon. To this was added benzyl bromide (8.42 mol) slowly over 1 hour, with a slight exotherm, and the reaction mixture stirred overnight at ambient temperature. The reaction was quenched cautiously with ammonium chloride solution (5 L) followed by water (35 L). The aqueous suspension was extracted with DCM (1×3 L and 2×5 L). The combined extracts were washed with water (10 L) and dried overnight (MgSO₄). The solution was evaporated in vacuo, and the crude product chromatographed in 3 batches (flash column, 3×2 kg silica, eluting with a gradient consisting of hexane containing 10% DCM, to neat DCM, to DCM containing 50% ethyl acetate) to eliminate starting material. The crude eluant was further chromatographed in 175 g batches (Amicon HPLC, 5 kg normal-phase silica, eluting with isohexane containing 20% v/v of ethyl acetate) to give the desired compound (21% yield). ¹H NMR δ (d₆-DMSO): 3.8 (s, 3H), 5.1 (s, 2H), 6.65 (m, 1H), 7.0 (m, 1H), 7.05 (m, 1H), 7.3-7.5 (m, 5H), 9.85 (br s, 1H).

### (2R)-1-{[(1,1-Dimethylethyl)(dimethyl)silyl]oxy}propan-2-ol

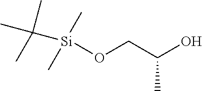

tert-Butyl(dimethyl)silyl chloride (5.90 g, 39.5 mmol) was added to a solution of (2R)-propane-1,2-diol (3.00 g, 39.5 mmol) in DCM (100 mL) followed by diisopropylethylamine (7.10 g, 55.3 mmol) and the reaction was stirred under argon for 72 h. The reaction was diluted with diethyl ether (500 mL) and water (140 mL) and the organic layer was separated then dried (MgSO₄), filtered and evaporated. Purification by column chromatography, eluting with 1:15 to 1:10 ethyl acetate:hexane, afforded the title compound as a colourless oil (6.00 g, 80%). ¹H NMR δ (CDCl₃): 0.10 (m, 6H), 0.92 (s, 9H), 1.14 (d, 3H), 2.42 (d, 1H), 3.38 (dd, 1H), 3.60 (dd, 1H), 3.82 (m, 1H). The data matched that reported in the literature (J. Org. Chem., 1998, 53, 2300).

### EXAMPLE 2

### 3-{[2-Fluoro-1-(fluoromethyl)ethyl]oxy}-5-[(4-methyl-5-oxo-2,3,4,5-tetrahydropyrido[3,2-f][1,4]oxazepin-8-yl)oxy]-N-(1-methyl-1H-pyrazol-3-yl)benzamide

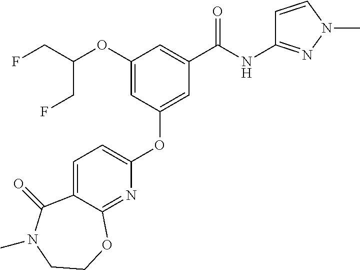

A mixture of 3-{[2-fluoro-1-(fluoromethyl)ethyl]oxy}-5-hydroxy-N-(1-methyl-1H-pyrazol-3-yl)benzamide (0.21 g, 0.67 mmol), 8-chloro-4-methyl-3,4-dihydropyrido[3,2-f][1,4]oxazepin-5(2H)-one (144 mg, 0.67 mmol) and potassium carbonate (187 mg, 1.74 mmol) in acetonitrile (5 mL) was stirred in a ‘Biotage initiator Microwave’ at 120° C. for 9 hours. The solvent was removed in vacuo and ethyl acetate (50 mL) added. The residue was washed with water (20 mL), brine (50 mL), dried (MgSO₄), filtered and the solvent removed in vacuo to give a yellow oil which was chromatographed on silica, eluting with a gradient of 50-100% ethyl acetate in isohexane, to give the desired compound (183 mg). ¹H NMR δ (CDCl₃): 3.21 (s, 3H), 3.66-3.70 (m, 2H), 3.73 (s, 3H), 4.50 (m, 2H), 4.62 (m, 2H), 4.73 (m, 3H), 6.72 (d, 1H), 6.79 (d, 1H), 6.95 (t, 1H), 7.26-7.31 (m, 2H), 7.37 (m, 1H), 8.56 (d, 1H), 9.14 (s, 1H); m/z 488 (M+H)⁺

The following compounds were prepared in an analogous fashion from the appropriate phenol and chloro-heterocycle.

| Example | Structure | m/z | NMR |
|---|---|---|---|
| 2a* | 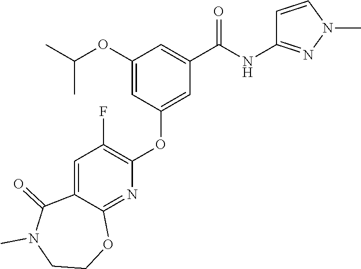 | 470 (M + H)⁺ | ¹H NMR δ (CDCl₃): 1.33 (d, 6H), 3.19 (s, 3H), 3.65 (t, 2H), 3.73 (s, 3H), 4.44 (t, 2H), 4.56 (septet, 1H), 6.79 (d, 1H), 6.86 (t, 1H), 7.17 (t, 1H), 7.26 (m, 2H), 8.33 (d, 1H), 8.93 (s, 1H) |
| 2b* | 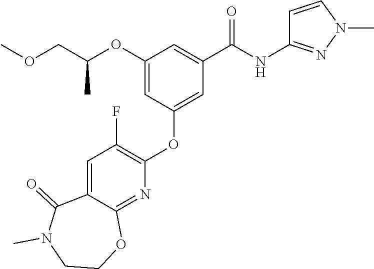 | 500 (M + H)⁺ | ¹H NMR δ (CDCl₃): 1.24 (d, 3H), 3.12 (s, 3H), 3.32 (s, 3H), 3.39-3.53 (m, 2H), 3.58 (t, 2H), 3.65 (s, 3H), 4.37 (t, 2H), 4.49 (sextet, 1H), 6.71 (d, 1H), 6.85 (t, 1H), 7.13 (t, 1H), 7.19 (d, 1H), 7.25 (t, 1H), 8.26 (d, 1H), 9.01 (s, 1H) |
| 2c* | 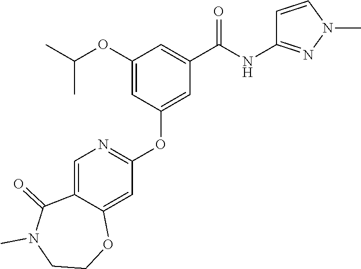 | 452 (M + H)⁺ | ¹H NMR δ (CDCl₃): 1.35 (d, 6H), 3.21 (s, 3H), 3.67 (t, 2H), 3.79 (s, 3H), 4.51 (t, 2H), 4.59 (septet, 1H), 6.38 (s, 1H), 6.79 (d, 1H), 6.83 (t, 1H), 7.13 (t, 1H), 7.25-7.28 (m, 2H), 8.46 (s, 1H), 8.90 (s, 1H) |

*Stirred in the microwave reactor for 3 hours at 160° C.

The preparation of 8-chloro-4-methyl-3,4-dihydropyrido[3,2-f][1,4]oxazepin-5(2H)-one, used in the synthesis of Example 2, is described below:

### 8-Chloro-4-methyl-3,4-dihydropyrido[3,2-f][1,4]oxazepin-5(2H)-one

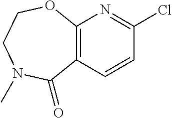

Sodium hydride (60% dispersion in oil) (432 mg, 10.77 mmol) was added to a solution of 2,6-dichloro-N-(2-hydroxyethyl)-N-methylpyridine-3-carboxamide (1.22 g, 4.90 mmol) in THF (100 mL) and the mixture refluxed for 150 minutes. The reaction mixture was cooled and added to iced water (150 mL) then extracted into ethyl acetate (2×100 mL) and washed with saturated aqueous sodium bicarbonate solution (80 mL), brine (80 mL), dried (Na₂SO₄), filtere and reduced to give a white solid. This residue was chromatographed on silica, eluting with 0-100% ethyl acetate in isohexane, to give the desired compound (0.43 g). ¹H NMR δ (CDCl₃): 3.25 (s, 3H), 3.71 (m, 2H), 4.61 (m, 2H), 7.15 (d, 1H), 8.44 (d, 1H); m/z 213 (M+H)⁺

The chloro-heterocycles used in the synthesis of Examples 2a-2c were prepared in an analogous fashion from the corresponding hydroxyl-containing compounds.

| Structure | m/z | NMR |
|---|---|---|
| 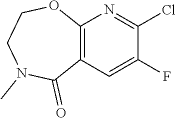 | | ¹H NMR δ (CDCl₃): 3.24 (s, 3H), 3.71 (t, 2H), 4.59 (t, 2H), 8.26 (d, 1H) |
| 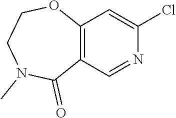 | 213 (M + H)⁺ | ¹H NMR δ (CDCl₃): 3.14 (s, 3H), 3.60 (m, 2H), 4.46 (m, 2H), 6.83 (s, 1H), 8.93 (s, 1H) |

The preparation of 2,6-dichloro-N-(2-hydroxyethyl)-N-methylpyridine-3-carboxamide, used in the synthesis of Example 2, is described below:

### 2,6-Dichloro-N-(2-hydroxyethyl)-N-methylpyridine-3-carboxamide

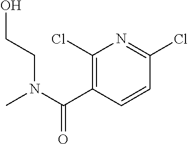

Oxalyl chloride (1.43 mL, 16.06 mmol) then DMF (2 drops) were added to a mixture of 2,6-dichloronicotinic acid (2.57 g, 13.39 mmol), 4M hydrogen chloride in dioxane (3.4 mL, 13.39 mmol) and DCM (50 mL). The reaction was stirred at RT for 2 hours, the volatiles removed in vacuo and the residue dissolved in DCM (25 mL). This solution was added dropwise to a mixture of 2-(methylamino)ethanol (1.19 mL, 14.72 mmol) and triethylamine (4.1 mL, 29.45 mmol) in DCM (25 mL) and the mixture stirred at RT for 20 hours. The reaction mixture was reduced in vacuo and ethyl acetate (100 mL) added to the residue. The organic solution was washed with water (100 mL), saturated sodium bicarbonate solution (50 mL), brine (50 mL), dried (MgSO₄), filtered and the solvent removed in vacuo to give a yellow oil. The residue was chromatographed on silica, eluting with a gradient of 50-100% ethyl acetate in isohexane, to give the desired compound (1.28 g). ¹H NMR δ (CDCl₃): 3.01 & 3.17 (2×s, 3H), 3.21-4.00 (m, 4H), 7.33 (m, 1H), 7.68 (m, 1H), the NMR spectrum was complicated due to the presence of rotamers; m/z 249, 251 (M+H)⁺

The hydroxyl-containing compounds used in the synthesis of Examples 2a-2c were prepared in an analogous fashion from 2-(methylamino)ethanol and the appropriate carboxylic acid.

| Structure | m/z | NMR |
|---|---|---|
| 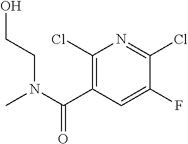 | | ¹H NMR δ (CDCl₃): 3.03 & 3.17 (2 × s, 3H), 3.21-4.00 (m, 4H), 7.56 & 7.61 (2 × d, 1H), the NMR spectrum was complicated due to the presence of rotamers |
| 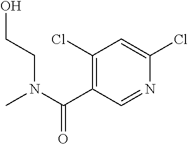 | 249 (M + H)⁺ | ¹H NMR δ (CDCl₃): 2.90 & 3.11 (2 × s, 3H), 3.17-3.91 (m, 4H), 7.35-7.40 (m, 1H), 8.28-8.33 (m, 1H), the NMR spectrum was complicated due to the presence of rotamers |

The preparation of 3-{[2-fluoro-1-(fluoromethyl)ethyl]oxy}-5-hydroxy-N-(1-methyl-1H-pyrazol-3-yl)benzamide, used in the synthesis of Example 2, is described below:

### 3-{[2-Fluoro-1-(fluoromethyl)ethyl]oxy}-5-hydroxy-N-(1-methyl-1H-pyrazol-3-yl)benzamide

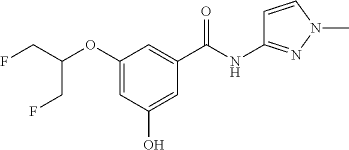

A solution of 3-{[2-fluoro-1-(fluoromethyl)ethyl]oxy}-N-(1-methyl-1H-pyrazol-3-yl)-5-[(phenylmethyl)oxy]benzamide (2.46 g, 6.13 mmol) and 10% by weigh palladium on carbon (0.246 g) in ethanol (100 mL) was allowed to stir at RT, under a hydrogen atmosphere overnight. The solution was filtered through Celite and the cake washed with methanol (100 mL). The solution was evaporated to give the desired compound (1.78 g). ¹H NMR δ (d₆-DMSO): 3.78 (s, 3H), 4.72 (m, 4H), 4.97 (m, 1H), 6.57 (d, 2H), 7.03 (s, 1H), 7.16 (s, 1H), 7.59 (s, 1H). m/z 312 (M+H)⁺

### 3-{[2-Fluoro-1-(fluoromethyl)ethyl]oxy}-N-1-methyl-1H-pyrazol-3-yl)-5-[(phenylmethyl)oxy]benzamide

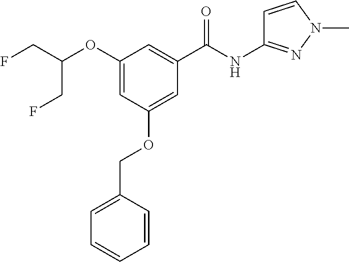

A solution 3-{[2-fluoro-1-(fluoromethyl)ethyl]oxy}-5-[(phenylmethyl)oxy]benzoic acid (3.00 g, 9.31 mmol), 3-amino-1-methylpyrazole (1.83 g, 18.6 mmol), HATU (4.60 g, 12.1 mmol) and DIPEA (3.25 mL, 18.6 mmol) in DMF (12 mL) was stirred at RT overnight. Water (150 mL) was added and the solution partitioned with ethyl acetate (250 mL). The ethyl acetate layer was separated, washed with brine and dried (MgSO₄), and evaporated to a residue which was chromatographed on silica, eluting with 50% ethyl acetate in isohexane, to give the desired product (2.46 g).

¹H NMR δ (CDCl₃): 3.69 (s, 3H), 4.57 (m, 5H), 5.00 (s, 2H), 6.70 (t, 1H), 6.74 (d, 1H), 7.01 (t, 1H), 7.08 (t, 1H), 7.21 (d, 1H), 7.30 (m, 5H), 8.68 (s, 1H); m/z 402 (M+H)⁺

### 3-{[2-Fluoro-1-(fluoromethyl)ethyl]oxy}-5-[(phenylmethyl)oxy]benzoic acid

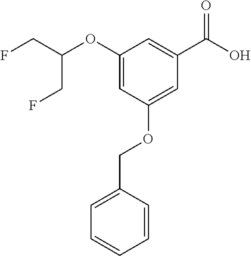

A solution of lithium hydroxide monohydrate (2.32 g, 55.1 mmol) in water (100 mL) was added to a solution of methyl 3-{[2-fluoro-1-(fluoromethyl)ethyl]oxy}-5-[(phenylmethyl)oxy]benzoate (7.41 g, 22.0 mmol) in THF (200 mL) and the mixture allowed to stir at RT overnight. The THF was removed in vacuo and the resulting solution partitioned between water (100 mL) and ethyl acetate (250 mL). The ethyl acetate layer was separated, washed with brine and dried (MgSO₄). The aqueous layer was then adjusted to pH 7 by addition of 1M hydrochloric acid and extracted with ethyl acetate (75 mL). The ethyl acetate layer was separated, washed with brine and dried (MgSO₄). The ethyl acetate layers were combined and evaporated to give the required product (6.404 g).

¹H NMR δ (d₆-DMSO): 4.74 (m, 4H), 5.08 (s, 2H), 6.67 (s, 1H), 6.67 (s, 1H), 7.23 (s, 1H), 7.37 (m, 5H). m/z 231 (M−H)⁻

### Methyl 3-{[2-fluoro-1-(fluoromethyl)ethyl]oxy}-5-[(phenylmethyl)oxy]benzoate

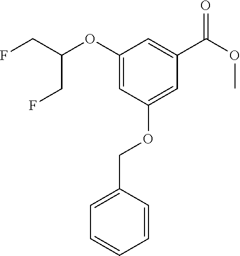

DIAD (7.63 mL, 38.7 mmol) was added in a drop wise fashion to a solution of methyl 3-hydroxy-5-{[phenylmethyl]oxy}benzoate (5.00 g, 19.4 mmol), 1,3-difluoropropan-2-ol (3 mL, 38.7 mmol), and triphenylphosphine (10.16 g, 38.7 mmol) in THF (100 mL) under an inert atmosphere at 0° C. The solution was allowed to reach RT and left to stir for 2 days. The THF was removed in vacuo and the residual oil slurried with a mixture of 20% ethyl acetate in isohexane. After allowing to stir for 90 minutes the mixture was filtered and the filtrate evaporated. The residual oil was chromatographed on silica, eluting with 30% ethyl acetate in isohexane, to give the desired compound (7.41 g). ¹H NMR δ (d₆-DMSO): 3.85 (s, 3H), 4.71 (m, 4H), 5.03 (m, 1H), 5.17 (s, 2H), 7.01 (t, 1H), 7.20 (m, 2H), 7.40 (m, 5H). m/z 335 (M−H)⁻

The preparation of methyl 3-hydroxy-5-{[phenylmethyl]oxy}benzoate was described earlier.

The preparation of 3-hydroxy-5-[(1-methylethyl)oxy]-N-(1-methyl-1H-pyrazol-3-yl)benzamide, used in the synthesis of Examples 2a and 2c, is described below:

### 3-Hydroxy-5-[(1-methylethyl)oxy]-N-(1-methyl-1H-pyrazol-3-yl)benzamide

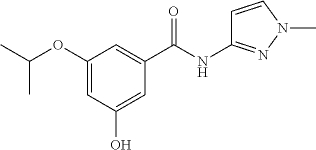

### 3-[(1-Methylethyl)oxy]-N-(1-methyl-1H-pyrazol-3-yl)-5-[(phenylmethyl)oxy]benzamide (51 g; 0.14 mol) was dissolved in methanol (500 mL) and THF (500 mL) then the flask was evacuated and purged with argon (3 times). 10% Palladium on carbon (5.1 g) was added and the flask was further evacuated and finally purged with hydrogen gas. The reaction mixture was stirred at RT for 20 hours. The reaction mixture was evacuated and purged with nitrogen (3 times). The catalyst was filtered off through celite, and the filtrate concentrated in vacuo. Ethyl acetate was added and filtered to give the desired compound. (30.5 g). A second crop of material was obtained in the same way (4.0 g).

¹H NMR δ (d₆-DMSO): 1.30 (d, 6H), 3.78 (s, 3H), 4.68 (sept, 1H), 6.47 (m, 1H), 6.60 (s, 1H), 6.94 (s, 1H), 7.05 (s, 1H), 7.60 (s, 1H), 10.63 (s, 1H). m/z 276 (M+H)⁺

### 3-[(1-Methylethyl)oxy]-N-(1-methyl-1H-pyrazol-3-yl)-5-[(phenylmethyl)oxy]benzamide

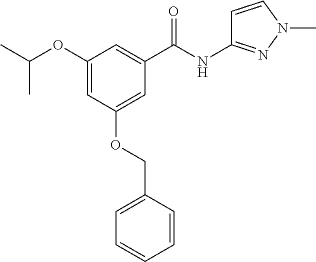

DMF (2 drops) was added to a solution of 3-[(1-methylethyl)oxy]-5-[(phenylmethyl)oxy]benzoic acid (40.0 g, 0.14 mol) and oxalyl chloride (14.6 mL, 0.17 mol) in DCM (700 mL) The mixture was stirred at RT for 4 hours and the DCM and excess oxalyl chloride evaporated in vacuo. The residual acid chloride was dissolved in DCM (300 mL) and added dropwise to 1-methyl-3-aminopyrazole (14.25 g, 0.147 mol) and triethylamine (41 mL, 0.29 mol) in DCM (300 mL), at 0° C. The mixture was stirred at RT for 24 hours. The DCM was evaporated in vacuo, and the residue partitioned between ethyl acetate (400 mL) and 1N hydrochloric acid (200 mL). The ethyl acetate layer was washed sequentially with saturated aqueous sodium hydrogen carbonate (200 mL) and brine (100 mL), dried (MgSO₄) and evaporated in vacuo. The residue was chromatographed on silica, eluting with a gradient of 50% ethyl acetate in isohexane, to give the desired compound (51 g). ¹H NMR δ (CDCl₃): 1.30 (d, 6H), 3.61 (s, 3H), 4.50 (sept, 1H), 5.01 (s, 2H), 6.66 (m, 1H), 6.88 (m, 1H), 7.00 (m, 1H), 7.06 (m, 1H), 7.24 (m, 1H), 7.39 (m, 5H), 9.50 (s, 1H). m/z 366 (M+H)⁺

### 3-[(1-Methylethyl)oxy]-5-[(phenylmethyl)oxy]benzoic acid

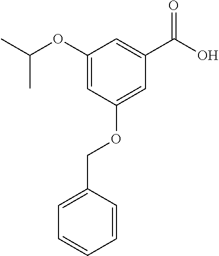

To a solution of methyl 3-[(1-methylethyl)oxy]-5-[(phenylmethyl)oxy]benzoate (37 g) in a 1:1 mixture of THF:methanol (300 mL) was added 4M sodium hydroxide solution (150 mL). The mixture was refluxed for 45 minutes, following which the organics were removed in vacuo. The aqueous was acidified to pH4 with hydrochloric acid (2M), and extracted with ethyl acetate. The organics were combined, washed with water and brine, dried (MgSO₄) and concentrated in vacuo to give the desired compound (33.5 g), which was used without further purification.

¹H NMR δ (d₆-DMSO): 1.26 (d, 6H), 4.59-4.69 (m, 1H), 5.15 (s, 2H), 6.80 (app t, 1H), 7.04 (m, 1H), 7.12 (m, 1H), 7.33 (app t, 1H), 7.40 (t, 2H), 7.46 (d, 2H), 12.95 (s, 1H)

### Methyl 3-[(1-methylethyl)oxy]-5-[(phenylmethyl)oxy]benzoate

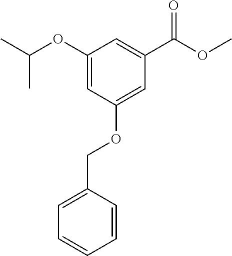

To a solution of methyl 3-hydroxy-5-[(1-methylethyl)oxy]benzoate (25 g) in DMF (250 mL) was added anhydrous potassium carbonate (297 mmol), and benzyl bromide (143 mmol). The mixture was stirred at 60° C. for 5 hours, then cooled to RT. The solvent was removed in vacuo and the residue partitioned between ethyl acetate and water. The organics were combined and washed with further water, brine, dried (MgSO₄) and concentrated in vacuo to give the desired compound (37 g) which was used without further purification.

¹H NMR δ (d₆-DMSO): 1.26 (d, 6H), 3.84 (s, 3H), 4.61-4.70 (m, 1H), 5.12 (s, 2H), 6.84 (t, 1H), 7.05 (app t, 1H), 7.12-7.15 (m, 1H), 7.31-7.37 (m, 1H), 7.40 (t, 2H), 7.46 (d, 2H)

### Methyl 3-hydroxy-5-[(1-methylethyl)oxy]benzoate

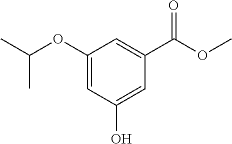

To a stirred solution of methyl 3,5-dihydroxybenzoate (0.1 mol) in DMF (180 mL) was added powdered potassium carbonate (0.2 mol) and 2-iodopropane (0.1 mol), and the resulting mixture stirred at RT for 16 hours. The reaction mixture was poured into water (1000 mL) and the mixture extracted with ether. The extracts were combined and washed sequentially with water (twice) and brine; the solution was dried (MgSO₄), filtered and evaporated in vacuo to give the crude product as a pale yellow oil (12.6 g). This was treated with toluene (40 mL) and allowed to stand overnight. The insoluble material (starting phenol) was removed by filtration, and the filtrate evaporated in vacuo. The resulting oil was chromatographed (2×90 g Biotage silica cartridges), eluting with hexane containing ethyl acetate (10% increasing to 15% v/v). The title compound was obtained as an oil (25% yield). TLC analysis matched that of a previous sample. ¹H NMR δ (d₆-DMSO): 1.2 (d, 6H), 3.8 (s, 3H), 4.5-4.6 (hept, 1H), 6.55 (m, 1H), 7.85 (m, 1H), 7.95 (m, 1H), 9.8 (s, 1H)

The preparation of 3-hydroxy-5-[(1S)-2-methoxy-(1-methylethyl)oxy]-N-(1-methyl-1H-pyrazol-3-yl)benzamide, used in the synthesis of Example 2b, is described below:

### 3-Hydroxy-5-[(1S)-2-methoxy-(1-methylethyl)oxy]-N-(1-methyl-1H-pyrazol-3-yl)benzamide

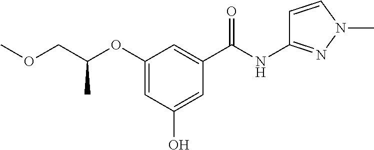

To a solution of 3-[(1S)-2-methoxy-(1-methylethyl)oxy]-N-(1-methyl-1H-pyrazol-3-yl)-5-[(phenylmethyl)oxy]benzamide (7.07 g) in THF (50 mL) and methanol (50 mL) was added 10% palladium on carbon (727 mg) as a slurry in THF (1 mL) and methanol (1 mL). The mixture was placed under vacuum and stirred under an atmosphere of hydrogen for 70 hours. The mixture was filtered through diatomaceous earth, and the diatomaceous earth washed with methanol (2×100 mL), and the combined organics were evaporated in vacuo. The residues were dissolved in ethyl acetate (10 mL), treated with isohexane (40 mL), the solid filtered off and washed with isohexane (50 mL) to afford the desired compound (5.17 g) which was used without further purification.

¹H NMR δ (d₆-DMSO): 1.22 (d, 3H), 3.28 (s, 3H, obscured by water), 3.38-3.53 (m, 2H), 3.76 (s, 3H), 4.65 (m, 1H), 6.44 (m, 1H), 6.54 (m, 1H), 6.93 (s, 1H), 7.04 (s, 1H), 7.57 (m, 1H), 9.63 (br s, 1H), 10.60 (s, 1H). m/z 306 (M+H)⁺, 304 (M−H)−

### 3-[(1S)-2-Methoxy-(1-methylethyl)oxy]-N-(1-methyl-1H-pyrazol-3-yl)-5-[(phenylmethyl)oxy]benzamide

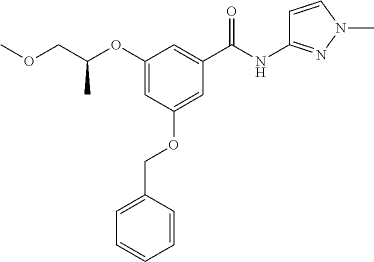

A solution of 3-[(1S)-2-methoxy-(1-methylethyl)oxy]-5-{[phenylmethyl]oxy}benzoic acid (8.73 g) in DCM (150 mL) was cooled to 0° C. Oxalyl chloride (4.81 mL) and DMF (0.15 mL) were slowly added with stirring. The mixture was allowed to warm to RT and stirred for 16 hours, then the organics were removed in vacuo, and the residues were azeotroped with toluene (75 mL). The crude material was dissolved in DCM (75 mL) and slowly added to a stirred suspension of 3-amino-1-methylpyrazole (3.35 g) and DIPEA (14.4 mL) in DCM (75 mL). The mixture was stirred at RT for 18 hours, before the organics were evaporated in vacuo and the residue dissolved in ethyl acetate (150 mL). The organics were washed with 1M aqueous hydrochloric acid (100 mL) and brine (50 mL), and dried (MgSO₄), before evaporation in vacuo to give crude material. This was chromatographed on a 200 g Biotage Flash 75 SiO₂ column (eluting with 30 to 90% ethyl acetate in isohexane), and evaporated in vacuo to afford the desired compound (7.07 g).

¹H NMR δ (d₆-DMSO): 1.23 (d, 3H), 3.28 (s, 3H, obscured by water), 3.40-3.52 (m, 2H), 3.77 (s, 3H), 4.70 (m, 1H), 5.03 (s, 2H), 6.56 (m, 1H), 6.71 (m, 1H), 7.18 (s, 1H), 7.24 (s, 1H), 7.32-7.47 (br m, 5H), 7.58 (m, 1H), 10.73 (s, 1H). m/z 396 (M+H)⁺.

### 3-[(1S)-2-Methoxy-(1-methylethyl)oxy]-5-{[phenylmethyl]oxy}benzoic acid

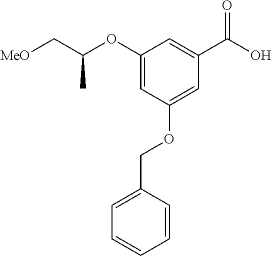

A solution of methyl 3-[(1S)-2-methoxy-(1-methylethyl)oxy]-5-{[phenylmethyl]oxy}benzoate (77.4 mmol) in a mixture of THF (232 mL) and methanol (232 mL) was treated with a solution of 2M sodium hydroxide (232 mmol), and the reaction mixture stirred for 4 hours at RT. The resulting solution was diluted with water (250 mL) and most of the organic solvent removed in vacuo. The resulting suspension was washed with diethyl ether (3×200 mL) and the organic washings discarded. The resulting aqueous solution was acidified to pH 4 with 2M hydrochloric acid solution and extracted with ethyl acetate (2×200 mL). The extracts were combined, washed with brine, dried (MgSO₄), and evaporated to give the desired compound (99% yield).

¹H NMR δ (d₆-DMSO): 1.20 (d, 3H), 3.46 (m, 2H), 4.64 (m, 1H), 5.15 (s, 2H), 6.83 (app t, 1H), 7.06 (s, 1H), 7.13 (s, 1H), 7.30-7.49 (m, 5H), 12.67 (br s, 1H)

### Methyl 3-[(1S)-2-methoxy-(1-methylethyl)oxy]-5-{[phenylmethyl]oxy}benzoate

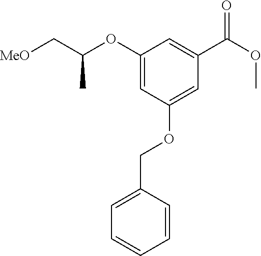

To a solution of methyl 3-hydroxy-5-{[phenylmethyl]oxy}benzoate (77.4 mmol) in THF was added polymer-supported triphenylphosphine (51.7 g of 3 mmol/g loading, 155 mmol) and (R)-(−)-1-methoxy-2-propanol (102 mmol). The stirred solution was blanketed with argon and cooled in an ice bath. A solution of DIAD (116 mmol) was added dropwise by syringe over 10 minutes. The solution was stirred for 20 minutes and filtered, washing the residue with THF (500 mL). The filtrate and washings were combined, and evaporated to give the desired compound which was used without further purification.

¹H NMR δ (d₆-DMSO): 3.26 (s, 3H), 3.44 (m, 2H), 3.82 (s, 3H), 4.63 (m, 1H), 5.14 (s, 2H), 6.85 (s, 1H), 7.05 (s, 1H), 7.11 (s, 1H), 7.30-7.47 (m, 5H)

The ¹H NMR spectrum also contained signals consistent with a small amount of bis(1-methylethyl)hydrazine-1,2-dicarboxylate.

The preparation of methyl 3-hydroxy-5-{[phenylmethyl]oxy}benzoate was described earlier.

### EXAMPLE 3

### 3-{[(1S)-1-Methyl-2-(methyloxy)ethyl]oxy}-N-(1-methyl-1H-pyrazol-3-yl)-5-[(4-oxo-5,6,7,8-tetrahydro-4H-[1,3]thiazolo[5,4-c]azepin-2-yl)oxy]benzamide

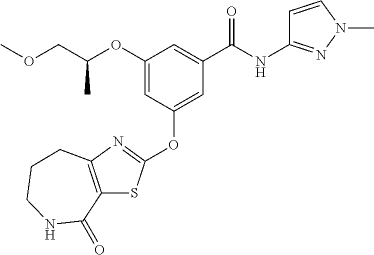

To a solution of 3-hydroxy-5-[(1S)-2-methoxy-(1-methylethyl)oxy]-N-(1-methyl-1H-pyrazol-3-yl)benzamide (153 mg, 0.5 mmol) and 2-chloro-5,6,7,8-tetrahydro-4H-[1,3]thiazolo[5,4-c]azepin-4-one (152 mg, 0.75 mmol) in acetonitrile (5 mL) was added cesium carbonate (489 mg, 1.5 mmol) and the stirred mixture heated at 160° C. in a Biotage Initiator Microwave reactor for 4 hours. The mixture was cooled to RT and pressure, the acetonitrile evaporated in vacuo, the residue partitioned between water (25 mL) and ethyl acetate (50 mL), the organic layer washed with brine, dried (MgSO₄) and evaporated to a residue which was chromatographed on silica, eluting with ethyl acetate, to give the desired compound (33 mg). ¹H NMR δ (CDCl₃): 1.2 (s, 3H), 1.3 (m, 2H), 2.05 (m, 2H), 2.95 (t, 2H), 3.3 (s, 3H), 3.4-3.55 (m, 2H), 3.75 (s, 3H), 4.55 (m, 1H), 6.8 (s, 1H), 7.05 (s, 1H), 7.1 (s, 1H), 7.25 (s, 1H), 7.4 (s, 1H), 7.45 (s, 1H) and 9.5 (s, 1H); m/z 472 (M+H)⁺.

The following compound was prepared in an analogous fashion from 2-chloro-5,6,7,8-tetrahydro-4H-[1,3]thiazolo[5,4-c]azepin-4-one and 3-hydroxy-5-[(1-methylethyl)oxy]-N-(1-methyl-1H-pyrazol-3-yl)benzamide.

| Example | Structure | m/z | NMR |
|---|---|---|---|
| 3a | 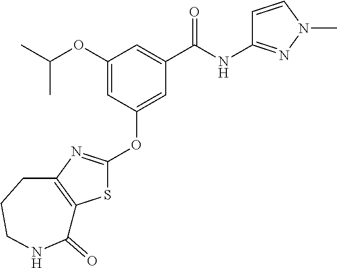 | 442 (M + H)⁺. | ¹H NMR δ (CDCl₃): 1.3 (s, 6H), 2.05 (m, 2H), 2.95 (t, 2H), 3.35 (m, 2H), 3.75 (s, 3H), 4.55 (m, 1H), 6.8 (s, 1H),7.05 (s, 1H), 7.25 (s, 1H), 7.4 (s, 1H), 7.6 (s, 1H), 7.8 (s, 1H) 9.9 (s, 1H). |

The preparation of 3-hydroxy-5-[(1S)-2-methoxy-(1-methylethyl)oxy]-N-(1-methyl-1H-pyrazol-3-yl)benzamide and 3-hydroxy-5-[(1-methylethyl)oxy]-N-(1-methyl-1H-pyrazol-3-yl)benzamide was described earlier.

2-Chloro-5,6,7,8-tetrahydro-4H-[1,3]thiazolo[5,4-c]azepin-4-one is a compound known in the literature [Russian Journal of General Chemistry (Translation of Zhurnal Obshchei Khimii), (2000), 70(5), 784].

### EXAMPLE 4

### 3-{[(1S)-1-Methyl-2-(methyloxy)ethyl]oxy}-5-[(4-methyl-5-oxo-2,3,4,5-tetrahydropyrido[3,4-f][1,4]oxazepin-8-yl)oxy]-N-(1-methyl-1H-pyrazol-3-yl)benzamide

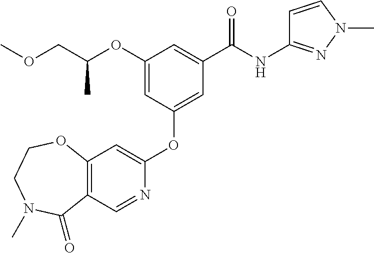

A mixture of 3-hydroxy-5-[(1S)-2-methoxy-(1-methylethyl)oxy]-N-(1-methyl-1H-pyrazol-3-yl)benzamide (0.11 g, 0.36 mmol), 8-chloro-4-methyl-3,4-dihydropyrido[3,4-f][1,4]oxazepin-5(2H)-one (77 mg, 0.36 mmol) and potassium carbonate (100 mg, 0.72 mmol) in acetonitrile (5 mL) was stirred in a microwave reactor at 160° C. for 5 hours. The mixture was concentrated in vacuo and ethyl acetate (50 mL) added to the residue. The organics were washed with water (40 mL), brine (40 mL), dried (MgSO₄), filtered and reduced in vacuo to give a yellow oil. The residue was chromatographed on silica, eluting with 50-100% ethyl acetate in isohexane, to give the desired compound (34 mg).

¹H NMR δ (CDCl₃): 1.32 (d, 3H), 3.20 (s, 3H), 3.40 (s, 3H), 3.46-3.61 (m, 2H), 3.66-3.69 (m, 2H), 3.72 (s, 3H), 4.47-4.51 (m, 2H), 4.53-4.61 (m, 1H), 6.37 (s, 1H), 6.79 (d, 1H), 6.89 (t, 1H), 7.17 (s, 1H), 7.26 (d, 1H), 7.32 (s, 1H), 8.88 (s, 1H), 9.03 (s, 1H); m/z 482 (M+H)⁺

The rearranged product 3-[(2,3-dimethyl-4-oxo-3,4-dihydro-2H-pyrido[3,4-e][1,3]oxazin-7-yl)oxy]-5-{[(1S)-1-methyl-2-(methyloxy)ethyl]oxy}-N-(1-methyl-1H-pyrazol-3-yl)benzamide, Example 4a, was also isolated from the above procedure.

### EXAMPLE 4a

### 3-[(2,3-Dimethyl-4-oxo-3,4-dihydro-2H-pyrido[3,4-e][1,3]oxazin-7-yl)oxy]-5-{[(1S)-1-methyl-2-(methyloxy)ethyl]oxy}-N-(1-methyl-1H-pyrazol-3-yl)benzamide

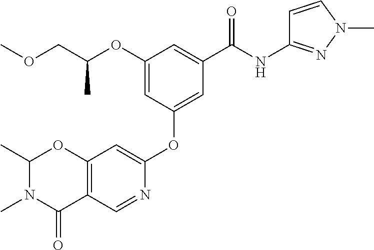

3-[(2,3-Dimethyl-4-oxo-3,4-dihydro-2H-pyrido[3,4-e][1,3]oxazin-7-yl)oxy]-5-{[(1S)-1-methyl-2-(methyloxy)ethyl]oxy}-N-(1-methyl-1H-pyrazol-3-yl)benzamide was isolated as a faster running fraction from the chromatography performed in Example 4 to give the title compound (10 mg). ¹H NMR δ (CDCl₃): 1.26 (d, 3H), 1.54 (d, 3H), 2.99 (s, 3H), 3.33 (s, 3H), 3.40-3.54 (m, 2H), 3.72 (s, 3H), 4.52 (sextet, 1H), 5.43 (q, 1H), 6.32 (s, 1H), 6.72 (d, 1H), 6.83 (t, 1H), 7.12 (t, 1H), 7.19-7.20 (m, 1H), 7.26 (t, 1H), 8.54 (s, 1H), 8.63 (s, 1H); m/z 482 (M+H)⁺

The preparation of 3-hydroxy-5-[(1S)-2-methoxy-(1-methylethyl)oxy]-N-(1-methyl-1H-pyrazol-3-yl)benzamide was described earlier.

The preparation of 8-chloro-4-methyl-3,4-dihydropyrido[3,4-f][1,4]oxazepin-5(2H)-one is described below:

### 8-Chloro-4-methyl-3,4-dihydropyrido[3,4-f][1,4]oxazepin-5(2H)-one

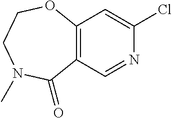

Sodium hydride (60% dispersion in oil) (583 mg, 14.57 mmol) was added to a solution of 4,6-dichloro-N-(2-hydroxyethyl)-N-methylpyridine-3-carboxamide (1.65 g, 6.62 mmol) in DMF (1100 mL) and the mixture stirred at RT for 72 hours. The reaction mixture was poured onto iced water (1500 mL) then extracted into ethyl acetate (2×400 mL). The organics were washed with saturated sodium bicarbonate solution (80 mL), brine (80 mL), dried (Na₂SO₄), filtered and reduced in vacuo to give a yellow oil. The residue was chromatographed on silica, eluting with 0-100% ethyl acetate in isohexane, to give the desired compound (0.56 g). ¹H NMR δ (CDCl₃): 3.14 (s, 3H), 3.58-3.62 (m, 2H), 4.42-4.46 (m, 2H), 6.83 (s, 1H), 8.93 (s, 1H); m/z 213 (M+H)⁺

### 4,6-Dichloro-N-(2-hydroxyethyl)-N-methylpyridine-3-carboxamide

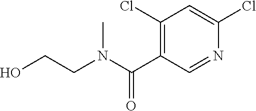

Oxalyl chloride (1.12 mL, 12.50 mmol), followed by DMF (2 drops), were added to a mixture of 4,6-dichloronicotinic acid (2 g, 10.42 mmol) in 4M HCl in dioxane (2.62 mL, 10.42 mmol) and DCM (40 mL). The reaction was stirred at RT for 2 hours, the volatiles removed in vacuo and the residue dissolved in DCM (20 mL). The solution was added dropwise to a mixture of 2-(methylamino)ethanol (0.93 mL, 11.46 mmol) and triethylamine (3.2 mL, 22.92 mmol) in DCM (20 mL) and the mixture stirred at RT for 20 hours. The mixture was concentrated in vacuo and ethyl acetate (100 mL) added to the residue. The organics were washed with water (100 mL), saturated sodium bicarbonate solution (50 mL), brine (50 mL), dried (MgSO₄), filtered and the solvent removed in vacuo to give a yellow oil. The residue was chromatographed on silica, eluting with a gradient of 50-100% ethyl acetate in isohexane, to give the desired compound (1.8 g).

¹H NMR δ (CDCl₃): 2.90 & 3.11 (2×s, 3H), 3.17-3.91 (m, 4H), 7.35-7.40 (m, 1H), 8.28-8.33 (m, 1H); m/z 249 (M+H)⁺

The preparation of 4,6-dichloronicotinic acid is described in the literature (European Journal of Organic Chemistry, (2001), 1371).

### EXAMPLE 5

### 3-[(7,7-Dimethyl-4-oxo-5,6,7,8-tetrahydro-4H-[1,3]thiazolo[5,4-c]azepin-2-yl)oxy]-5-{[(1S)-1-methyl-2-(methyloxy)ethyl]oxy}-N-(1-methyl-1H-pyrazol-3-yl)benzamide

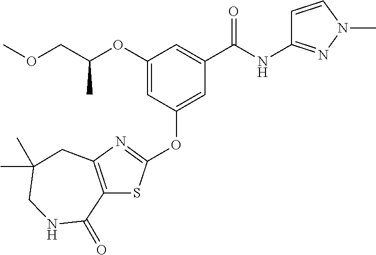

A mixture of 3-hydroxy-5-[(1S)-2-methoxy-(1-methylethyl)oxy]-N-(1-methyl-1H-pyrazol-3-yl)benzamide (0.25 g, 0.82 mmol), 2-chloro-7,7-dimethyl-5,6,7,8-tetrahydro-4H-[1,3]thiazolo[5,4-c]azepin-4-one (Russian Journal of General Chemistry 2001 1499) (285 mg, 1.23 mmol) and potassium carbonate (284 mg, 2.05 mmol) in acetonitrile (5 mL) was stirred in a microwave reactor at 150° C. for 5 hours. The solvent was removed in vacuo and ethyl acetate (50 mL) and water (50 mL) added. The ethyl acetate layer was washed with brine (50 mL), dried (MgSO₄), filtered and the solvent removed in vacuo to give a yellow oil. The residue was chromatographed on silica, eluting with 50-100% ethyl acetate in isohexane, to give the desired compound (106 mg).

¹H NMR δ (d₆-DMSO): 0.99 (s, 6H), 1.27 (d, 3H), 2.75 (s, 2H), 2.99 (d, 2H), 3.31 (s, 3H), 3.47-3.57 (m, 2H), 3.79 (s, 3H), 4.80 (sextet, 1H), 6.59 (d, 1H), 7.20 (t, 1H), 7.54 (t, 1H), 7.58 (t, 1H), 7.61 (d, 1H), 8.05 (t, 1H), 10.89 (s, 1H); m/z 500 (M+H)⁺

The following compounds were synthesised in an analogous fashion from the appropriate phenol and thiazolyl chloride:

| Example | Structure | m/z | NMR |
|---|---|---|---|
| 5a | 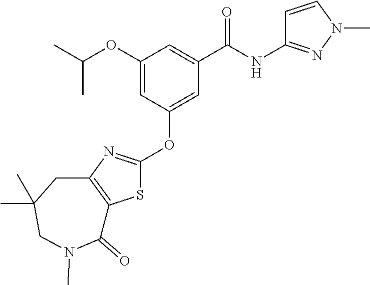 | 484 (M + H)⁺ | ¹H NMR δ (CDCl₃): 1.11 (s, 611), 1.38 (d, 6H), 2.77 (s, 2H), 3.19 (s, 3H), 3.27 (s, 2H), 3.77 (s, 3H), 4.60 (septet, 1H), 6.83 (d, 1H), 6.98 (t, 1H), 7.31-7.31 (m, 1H), 7.33-7.35 (m, 2H), 8.98 (s, 1H) |
| 5b | 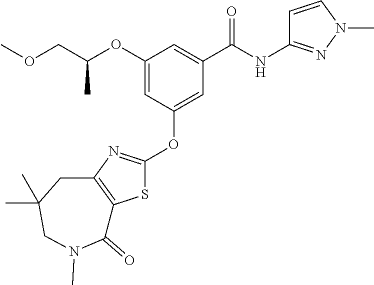 | 514 (M + H)⁺ | ¹H NMR δ (CDCl₃): 1.01 (s, 6H), 1.25 (d, 3H), 2.67 (s, 2H), 3.08 (s, 3H), 3.17 (s, 2H), 3.33 (s, 3H), 3.39-3.54 (m, 2H), 3.69 (s, 3H), 4.51 (sextet, 1H), 6.72 (d, 1H), 6.95 (t, 1H), 7.20 (d, 1H), 7.26-7.29 (m, 2H), 8.65 (s, 1H) |
| 5c | 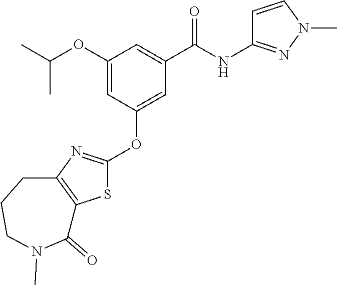 | 456 (M + H)⁺ | ¹H NMR δ (CDCl₃): 1.27 (d, 6H), 2.00- 2.07 (m, 2H), 2.88 (t, 2H), 3.06 (s, 3H), 3.41-3.47 (m, 2H), 3.67 (s, 3H), 4.50 (septet, 1H), 6.73 (d, 1H), 6.88 (d, 1H), 7.19-7.21 (m, 1H), 7.22-7.25 (m, 2H), 8.82 (s, 1H) |
| 5d | 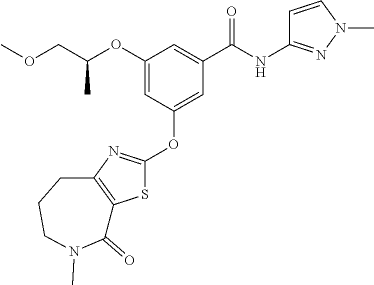 | 486 (M + H)⁺ | ¹H NMR δ (CDCl₃): 1.33 (d, 3H), 2.08- 2.15 (m, 2H), 2.96 (t, 2H), 3.14 (s, 3H), 3.41 (s, 3H), 3.48-3.61 (m, 4H), 3.75 (s, 3H), 4.58 (sextet, 1H), 6.81 (d, 1H), 7.03 (t, 1H), 7.28-7.29 (m, 1H), 7.35-7.38 (m, 2H), 8.98 (s, 1H) |

The preparations of 3-hydroxy-5-[(1S)-2-methoxy-(1-methylethyl)oxy]-N-(1-methyl-1H-pyrazol-3-yl)benzamide and 3-hydroxy-5-[(1-methylethyl)oxy]-N-(1-methyl-1H-pyrazol-3-yl)benzamide were described earlier.

The preparation of 2-chloro-7,7-dimethyl-5,6,7,8-tetrahydro-4H-[1,3]thiazolo[5,4-c]azepin-4-one was described in the literature (Russian Journal of General Chemistry, (2001), 1499).

The preparation of 2-chloro-5,7,7-trimethyl-5,6,7,8-tetrahydro-4H-[1,3]thiazolo[5,4-c]azepin-4-one is described below:

### 2-Chloro-5,7,7-trimethyl-5,6,7,8-tetrahydro-4H-[1,3]thiazolo[5,4-c]azepin-4-one

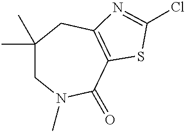

Sodium hydride (60% dispersion in oil) (86 mg, 2.15 mmol) was added to a solution of 2-chloro-7,7-dimethyl-5,6,7,8-tetrahydro-4H-[1,3]thiazolo[5,4-c]azepin-4-one (0.45 g, 1.95 mmol) in THF (10 mL) at 0° C., under argon. The mixture was allowed to warm to RT, methyl iodide (0.14 mL, 2.15 mmol) added, and the reaction stirred at RT for 24 hours. The mixture was poured onto iced water (50 mL) and extracted with ethyl acetate (50 mL). The organics were washed with brine (50 mL), dried (MgSO₄), filtered and the solvent removed in vacuo to give a yellow oil. The residue was chromatographed on silica, eluting with 30-60% ethyl acetate in isohexane, to give the desired compound (0.44 g).

¹H NMR δ (CDCl₃): 1.04 (s, 6H), 2.75 (s, 2H), 3.10 (s, 3H), 3.15 (s, 2H); m/z 245 (M+H)⁺

2-Chloro-5-methyl-5,6,7,8-tetrahydro-4H-[1,3]thiazolo[5,4-c]azepin-4-one was prepared in an analogous fashion from 2-chloro-5,6,7,8-tetrahydro-4H-[1,3]thiazolo[5,4-c]azepin-4-one.

| Structure | m/z | NMR |
|---|---|---|
| 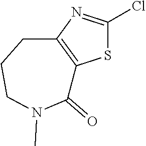 | 217 (M + H)⁺ | ¹H NMR δ (CDCl₃): 2.04-2.11 (m, 2H), 3.01 (t, 2H), 3.08 (s, 3H), 3.45 (m, 2H) |

### EXAMPLE 6

### 3-{[(1S)-1-Methyl-2-(methyloxy)ethyl]oxy}-5-[(4-methyl-5-oxo-2,3,4,5-tetrahydropyrido[2,3-f][1,4]oxazepin-8-yl)oxy]-N-(5-methylpyrazin-2-yl)benzamide

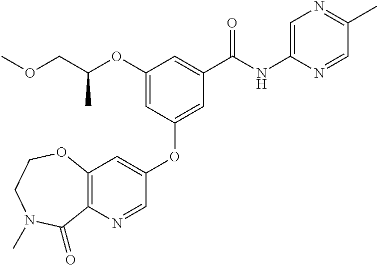

A mixture of 3-hydroxy-5-{[(1S)-1-methyl-2-(methyloxy)ethyl]oxy}-N-(5-methylpyrazin-2-yl)benzamide (0.185 g, 0.58 mmol), 8-fluoro-4-methyl-3,4-dihydropyrido[2,3-f][1,4]oxazepin-5(2H)-one (242 mg, 1.23 mmol) and potassium carbonate (202 mg, 1.46 mmol) in acetonitrile (5 mL) was stirred in a microwave reactor at 160° C. for 6 hours. The solvent was removed in vacuo and ethyl acetate (50 mL) added. The mixture was washed with water (50 mL), brine (50 mL), dried (MgSO₄), filtered and the solvent removed in vacuo. The residue was chromatographed on silica, eluting with 0-10% methanol in ethyl acetate, to give the desired compound (47 mg).

¹H NMR δ (CDCl₃): 1.26 (d, 3H), 2.47 (s, 3H), 3.17 (s, 3H), 3.32 (s, 3H), 3.41-3.52 (m, 2H), 3.52-3.56 (m, 2H), 4.37 (t, 2H), 4.51-4.58 (m, 1H), 6.77 (t, 1H), 6.84 (d, 1H), 7.14 (t, 1H), 7.28 (t, 1H), 8.05 (s, 1H), 8.23 (s, 1H), 8.51 (s, 1H), 9.44 (s, 1H); m/z 494 (M+H)⁺

The preparation of 8-fluoro-4-methyl-3,4-dihydropyrido[2,3-f][1,4]oxazepin-5(2H)-one is described below.

### 8-Fluoro-4-methyl-3,4-dihydropyrido[2,3-f][1,4]oxazepin-5(2H)-one

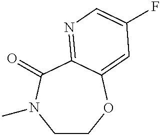

Sodium hydride (60% dispersion in mineral oil) (1.63 g, 40.7 mmol) was added to a solution of 3,5-difluoro-N-(2-hydroxyethyl)-N-methylpyridine-2-carboxamide (2 g, 9.25 mmol) in DMF (1000 mL) and the reaction stirred at RT for 6 days. The mixture was added to iced water (1000 mL) and extracted into ethyl acetate (2×500 mL). The organics were washed with brine (100 mL), dried (Na₂SO₄) and reduced in vacuo to give the desired material as a yellow oil (1.1 g). m/z 197 (M+H)⁺

### 3,5-Difluoro-N-(2-hydroxyethyl)-N-methylpyridine-2-carboxamide

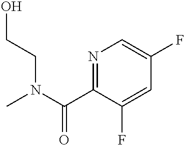

Oxalyl chloride (3.35 mL, 37.1 mmol) then DMF (2 drops) were added to a mixture of 3,5-difluoropyridine-2-carboxylic acid (5 g, 31.4 mmol) in 4M hydrogen chloride in dioxane (7.89 mL, 31.4 mmol) and DCM (80 mL). The mixture was stirred at RT for 2 hours, the volatiles removed in vacuo and the residue dissolved in DCM (40 mL). The solution was added dropwise to a mixture of 2-(methylamino)ethanol (2.8 mL, 34.6 mmol) and triethylamine (9.64 mL, 69.1 mmol) in DCM (40 mL) and the mixture stirred at RT for 20 hours. The mixture was reduced in vacuo and partitioned between ethyl acetate (100 mL) and water (100 mL). The organics were washed with citric acid (50 mL), saturated aqueous sodium bicarbonate solution (50 mL), brine (50 mL), dried (MgSO₄), filtered and the solvent removed in vacuo. The residue was chromatographed on silica, eluting with a gradient of 50-100% ethyl acetate in isohexane, to give the desired compound (5.75 g). ¹H NMR δ (CDCl₃): 3.04 & 3.20 (2×s, 3H), 3.40-3.98 (m, 4H), 7.27-7.39 (m, 1H), 8.31 & 8.39 (2×s, 1H); m/z 217 (M+H)⁺

The preparation of 3-hydroxy-5-{[(1S)-1-methyl-2-(methyloxy)ethyl]oxy}-N-(5-methylpyrazin-2-yl)benzamide is described below:

### 3-Hydroxy-5-{[(1S)-1-methyl-2-(methyloxy)ethyl]oxy}-N-(5-methylpyrazin-2-yl)benzamide

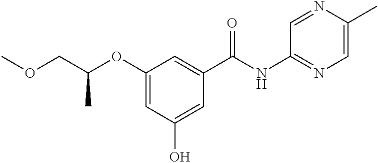

10% Palladium on charcoal (700 mg) was added to a solution of 3-{[(1S)-1-methyl-2-(methyloxy)ethyl]oxy}-N-(5-methylpyrazin-2-yl)-5-[(phenylmethyl)oxy]benzamide (7.0 g, 17.2 mmol) in ethanol (125 mL) and the mixture stirred at RT under a hydrogen atmosphere for 4 hours. The catalyst was removed by filtration and the ethanol evaporated in vacuo. The residue was crystallised from ethyl acetate to give the desired compound (4.22 g). ¹H NMR δ (CDCl₃): 1.25 (d, 3H), 2.5 (s, 3H), 3.3 (s, 3H), 3.4-3.5 (m, 2H), 4.5 (m, 1H), 6.3 (br, 1H), 6.55 (s, 1H), 6.9 (s, 1H), 6.95 (s, 1H), 8.05 (s, 1H), 8.45 (s, 1H) and 9.5 (s, 1H). m/z 318 (M+H)⁺.

### 3-{[(1S)-1-Methyl-2-(methyloxy)ethyl]oxy}-N-(5-methylpyrazin-2-yl)-5-[(phenylmethyl)oxy]benzamide

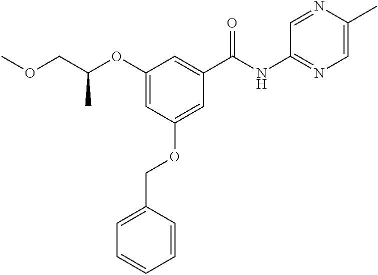

Oxalyl chloride (2.1 mL, 24.0 mmol) was added to a solution of 3-[(1S)-2-methoxy-(1-methylethyl)oxy]-5-{[phenylmethyl]oxy}benzoic acid (6.32 g, 20.0 mmol) in DCM (100 mL) and the mixture stirred at RT for 4 hours. The mixture was evaporated in vacuo to a residue, which was taken up in DCM (25 mL) and added to a stirred mixture of 2-amino-5-methylpyrazine (2.29 g, 21.0 mmol) and pyridine (1.94 mL, 24.0 mmol) in DCM (100 mL) at 5° C.-10° C. The mixture was stirred at RT for 18 hours, the DCM evaporated in vacuo. The residue was partitioned between water (50 mL) and ethyl acetate (150 mL), the organic layer washed with brine, dried (MgSO₄) and evaporated to a residue, which was chromatographed on silica, eluting with 50% ethyl acetate in isohexane, to give the desired compound (7.0 g). ¹H NMR δ (CDCl₃): 1.3 (d, 3H), 2.5 (s, 3H), 3.3 (s, 3H), 3.4-3.5 (m, 2H), 4.5 (m, 1H), 5.0 (s, 2H), 6.7 (s, 1H), 7.0 (s, 1H), 7.05 (s, 1H), 7.35 (m, 5H), 8.05 (s, 1H), 8.3 (s, 1H) and 9.5 (s, 1H). m/z 408 (M+H)⁺.

The preparation of 2-amino-5 methylpyrazine is described in the literature [Tett lett. 2002, 9287].

The preparation of 3-[(1S)-2-methoxy-(1-methylethyl)oxy]-5-{[phenylmethyl]oxy}benzoic acid was described earlier.

### EXAMPLE 7

### N-(1-Methyl-1H-pyrazol-3-yl)-3-[(3S)-tetrahydrofuran-3-yloxy]-5-[(5,7,7-trimethyl-4-oxo-5,6,7,8-tetrahydro-4H-[1,3]thiazolo[5,4-c]azepin-2-yl)oxy]benzamide

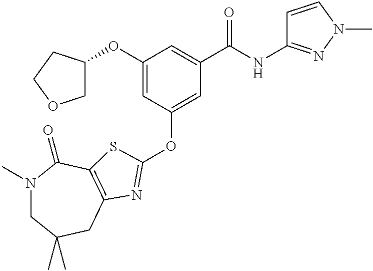

A mixture of 3-hydroxy-N-(1-methyl-1H-pyrazol-3-yl)-5-[(3S)-tetrahydrofuran-3-yloxy]benzamide (0.12 g, 0.4 mmol), 2-chloro-5,7,7-trimethyl-5,6,7,8-tetrahydro-4H-[1,3]thiazolo[5,4-c]azepin-4-one (97 mg, 0.4 mmol) and potassium carbonate (110 mg, 0.79 mmol) in acetonitrile (5 mL) was stirred in a microwave reactor at 150° C. for 5 hours. The solvent was removed in vacuo and ethyl acetate (50 mL) added to the residue. The mixture was washed with water (20 mL), brine (50 mL), dried (MgSO₄), filtered and the solvent removed in vacuo. The residue was chromatographed on silica, eluting with a gradient of 50-100% ethyl acetate in isohexane, to give the desired compound (133 mg).

¹H NMR δ (CDCl₃): 1.01 (s, 6H), 2.03-2.22 (m, 2H), 2.67 (s, 2H), 3.08 (s, 3H), 3.17 (s, 2H), 3.70 (s, 3H), 3.79-3.97 (m, 4H), 4.86-4.92 (m, 1H), 6.73 (d, 1H), 6.91 (t, 1H), 7.21-7.24 (m, 2H), 7.27-7.29 (m, 1H), 8.77 (s, 1H); m/z 512 (M+H)⁺

The preparation of 2-chloro-5,7,7-trimethyl-5,6,7,8-tetrahydro-4H-[1,3]thiazolo[5,4-c]azepin-4-one was described earlier

The preparation of 3-hydroxy-N-(1-methyl-1H-pyrazol-3-yl)-5-[(3S)-tetrahydrofuran-3-yloxy]benzamide is described below:

### 3-Hydroxy-N-(1-methyl-1H-pyrazol-3-yl)-5-[(3S)-tetrahydrofuran-3-yloxy]benzamide

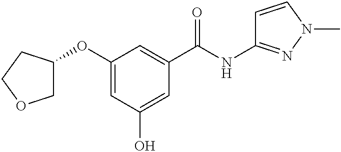

N-(1-Methyl-1H-pyrazol-3-yl)-3-[(phenylmethyl)oxy]-5-[(3S)-tetrahydrofuran-3-yloxy]benzamide (453 mg, 1.15 mmol) was dissolved in ethanol (5 mL) and ammonium formate (182 mg, 2.88 mmol) was added in one portion. The reaction was blanketed with argon and 10% Palladium on activated carbon (30 mg) was added. This mixture was heated to 140° C. for 10 minutes in a Smith Creator microwave. The catalyst was filtered off and the volatiles removed in vacuo to give the title product as a white solid (339 mg).

¹H NMR δ (CDCl₃): 2.06-2.14 (1H, m), 2.15-2.22 (1H, m), 3.72-3.73 (3H, s), 3.84-3.89 (1H, m), 3.92-3.98 (3H, m), 4.88 (1H, m), 6.53 (1H, t), 6.78 (1H, d), 6.89 (1H, s), 6.95 (1H, s), 7.28 (1H, d), 9.27 (1H, s); m/z 304 (M+H)⁺.

### N-(1-Methyl-1H-pyrazol-3-yl)-3-[(phenylmethyl)oxy]-5-[(3S)-tetrahydrofuran-3-yloxy]benzamide

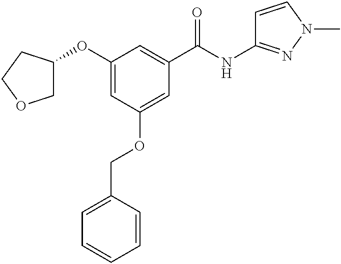

A suspension of 3-hydroxy-N-(1-methyl-1H-pyrazol-3-yl)-5-[(phenylmethyl)oxy]benzamide (450 mg, 1.39 mmol), (3R)-tetrahydrofuran-3-yl 4-methylbenzenesulfonate (507 mg, 2.09 mmol) and potassium carbonate (481 mg, 3.48 mmol) in acetonitrile (5 mL) was stirred in a Smith Creator microwave at 160° C. for 3 hours. The solvent was removed in vacuo and ethyl acetate added. The organics were washed with water (40 mL), brine (40 mL), dried (MgSO₄), filtered and the solvent removed in vacuo to give a yellow foam which was chromatographed on silica, eluting with a gradient of 0-100% ethyl acetate in isohexane, to give the title compound as a white foam (452 mg). ¹H NMR δ (CDCl₃): 2.09-2.14 (1H, m), 2.14-2.24 (1H, m), 3.68 (3H, s), 3.86-3.91 (1H, m), 3.94-3.98 (3H, m), 4.89 (1H, s), 5.03 (2H, s), 6.64 (1H, t), 6.85 (1H, s), 6.96 (1H, d), 7.07 (1H, t), 7.27 (1H, m), 7.33-7.41 (5H, m), 9.31 (1H, s); m/z 394 (M+H)⁺.

### (3R)-Tetrahydrofuran-3-yl 4-methylbenzenesulfonate

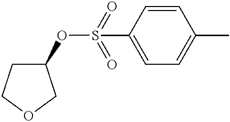

4-Toluene sulfonyl chloride (1.65 g, 8.63 mmol) was added to a solution of R-3-hydroxytetrahydrofuran (0.8 g, 9.08 mmol) and pyridine (0.88 mL, 10.9 mmol) in DCM (15 mL). The reaction was stirred at RT for 72 hours. Water (10 mL) and 1M hydrochloric acid (1 mL) were added and the mixture extracted with DCM (15 mL). The organic layer was washed with brine (20 mL), dried (MgSO₄), filtered and reduced in vacuo to give a yellow oil which was chromatographed on silica, eluting with a gradient of 0-50% ethyl acetate in isohexane, to give the desired compound (1.0 g). ¹H NMR δ (CDCl₃): 2.13 (m, 2H), 2.47 (s, 3H), 3.80-3.95 (m, 4H), 5.15 (m, 1H), 7.37 (d, 2H), 7.81 (d, 2H).

### 3-Hydroxy-N-(1-methyl-1H-pyrazol-3-yl)-5-[(phenylmethyl)oxy]benzamide

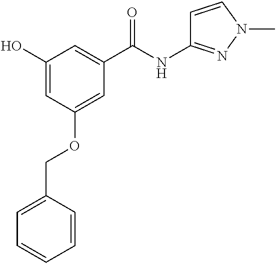

A suspension of N-(1-methyl-1H-pyrazol-3-yl)-3,5-bis[(phenylmethyl)oxy]benzamide (1.0 g, 2.42 mmol) was dissolved in ethanol (12 mL) and ammonium formate (229 mg, 3.63 mmol) was added in one portion. The reaction was blanketed with argon and 10% Palladium on activated carbon (10 mg) was added. This mixture was heated to 140° C. for 5 minutes in a Smith Creator microwave. The catalyst was filtered off and the volatiles removed in vacuo, the residue was chromatographed on silica, eluting with a gradient of 30-100% ethyl acetate in iso-hexane, to give the title compound as a white solid (378 mg). ¹H NMR δ (d₆-DMSO): 3.78 (3H, s), 5.13 (2H, s), 6.55-6.57 (2H, m), 6.99 (1H, s), 7.17 (1H, s), 7.34-7.48 (5H, m), 7.60 (1H, d), 9.74 (1H, s), 10.70 (1H, s); m/z 324 (M+H)⁺.

### N-(1-Methyl-1H-pyrazol-3-yl)-3,5-bis[phenylmethyl)oxy]benzamide

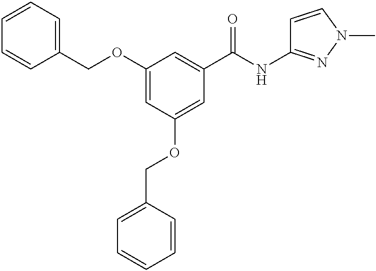

Oxalyl chloride (7.71 mL, 89.7 mmol) was added dropwise to a suspension of 3,5-dibenzyloxybenzoic acid (20.0 g, 59.8 mmol) in DCM (0.5 L) under argon. The reaction was stirred at RT for 6 hours after which time the volatiles were removed in vacuo. The residue was taken up in DCM (300 mL) and a solution of 1-methyl-1H-pyrazol-3-amine (5.81 g, 59.8 mmol) in DCM (50 mL) was added dropwise. The resulting solution was stirred for 16 hours at RT after which time a precipitate had formed. The solid was isolated by filtration and recrystallised from ethanol to give the title compound as a white solid (14.8 g). ¹H NMR δ (d₆-DMSO): 3.84 (3H, s), 5.17 (4H, s), 6.59 (1H, d), 6.84 (1H, t), 7.33-7.46 (12H, m), 7.62 (1H, d), 10.83 (1H, s); m/z 414 (M+H)⁺.

### EXAMPLE 8

### 3-({(1S)-2-[(Difluoromethyl)oxy]-1-methylethyl}oxy)-5-[(4-methyl-5-oxo-2,3,4,5-tetrahydropyrido[3,4-f][1,4]oxazepin-8-yl)oxy]-N-(1-methyl-1H-pyrazol-3-yl)benzamide

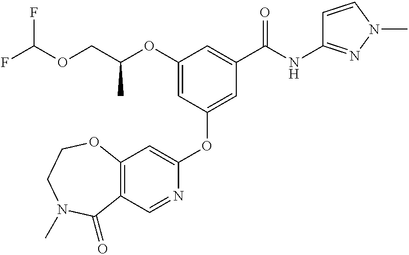

A mixture of 3-({(1S)-2-[(difluoromethyl)oxy]-1-methylethyl}oxy)-5-hydroxy-N-(1-methyl-1H-pyrazol-3-yl)benzamide (0.1 g, 0.29 mmol), 8-chloro-4-methyl-3,4-dihydropyrido[3,4-f][1,4]oxazepin-5(2H)-one (94 mg, 0.44 mmol) and potassium carbonate (81 mg, 0.59 mmol) in acetonitrile (5 mL) was stirred in a microwave reactor at 160° C. for 6 hours. The mixture was reduced in vacuo and the residue partitioned between ethyl acetate (50 mL) and water (50 mL). The organics were washed with brine (50 mL), dried (MgSO₄), filtered and reduced in vacuo to give a brown oil which was chromatographed on silica, eluting with 0-10% methanol in ethyl acetate, to give the desired compound (34 mg).

¹H NMR δ (CDCl₃): 1.40 (d, 3H), 3.23 (s, 3H), 3.70 (t, 2H), 3.83 (s, 3H), 3.94-4.06 (m, 2H), 4.53 (t, 2H), 4.67 (sextet, 1H), 6.28 (t, 1H), 6.43 (s, 1H), 6.82 (d, 1H), 6.91 (t, 1H), 7.22 (s, 1H), 7.31 (d, 1H), 7.34 (t, 1H), 8.58 (s, 1H), 8.92 (s, 1H); m/z 518 (M+H)⁺

The preparation of 8-chloro-4-methyl-3,4-dihydropyrido[3,4-f][1,4]oxazepin-5(2H)-one was described earlier.

The preparation of 3-({(1S)-2-[(difluoromethyl)oxy]-1-methylethyl}oxy)-5-hydroxy-N-(1-methyl-1H-pyrazol-3-yl)benzamide is described below:

### 3-({(1S)-2-[(Difluoromethyl)oxy]-1-methylethyl}oxy)-5-hydroxy-N-(1-methyl-1H-pyrazol-3-yl)benzamide

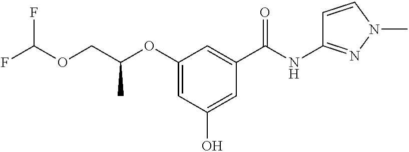

3-({(1S)-2-[(Difluoromethyl)oxy]-1-methylethyl}oxy)-N-(1-methyl-1H-pyrazol-3-yl)-5-[(phenylmethyl)oxy]benzamide (0.1 g, 0.23 mmol) was dissolved in ethanol (3 mL) and THF (3 mL) and the flask evacuated and purged with argon (3 times). 10% Palladium on carbon (0.01 g) was added and the flask further evacuated and finally purged with hydrogen gas. The reaction mixture was stirred at RT for 20 hours until completion. The reaction mixture was evacuated and purged with nitrogen (3 times). The catalyst was filtered off through celite and the filtrate concentrated in vacuo to give the desired compound (70 mg). ¹H NMR δ (CDCl₃): 1.28 (d, 3H), 3.71 (s, 3H), 3.80-3.95 (m, 2H), 4.51 (sextet, 1H), 5.96-6.36 (t, 1H), 6.53 (s, 1H), 6.73 (s, 1H), 6.91 (s, 1H), 6.96 (s, 1H), 7.22 (s, 1H), 8.83 (s, 1H). m/z 342 (M+H)⁺.

### 3-({(1S)-2-[(Difluoromethyl)oxy]-1-methylethyl}oxy)-N-(1-methyl-1H-pyrazol-3-yl)-5-[(phenylmethyl)oxy]benzamide

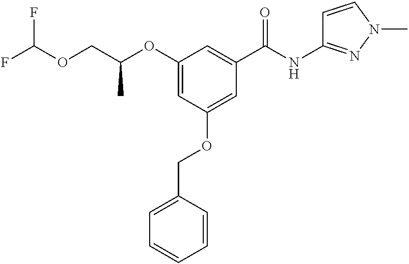

DIPEA (0.198 mL, 1.14 mmol) was added to a mixture of 3-({(1S)-2-[(difluoromethyl)oxy]-1-methylethyl}oxy)-5-[(phenylmethyl)oxy]benzoic acid (0.1 g, 0.28 mmol), 3-amino-1-methylpyrazole (39 mg, 0.4 mmol) and HATU (0.227 g, 0.6 mmol) in DMF (3 mL) and stirred at RT for 20 hours. Ethyl acetate (30 mL) was added and the mixture washed with water (30 mL), brine (30 mL), dried (MgSO₄), filtered and reduced in vacuo to give a yellow oil which was chromatographed on silica, eluting with a gradient of 0-100% ethyl acetate in isohexane, to give the desired compound (0.1 g).

¹H NMR δ (CDCl₃): 1.36 (d, 3H), 3.68 (s, 3H), 3.82-3.95 (m, 2H), 4.48 (sex, 1H), 5.00 (s, 2H), 6.19 (t, 1H), 6.63 (s, 1H), 6.73 (s, 1H), 6.93 (s, 1H), 7.03 (s, 1H), 7.28 (m, 1H), 7.35 (m, 5H), 8.59 (s, 1H). m/z 432 (M+H)⁺.

### 3-({(1S)-2-[(Difluoromethyl)oxy]-1-methylethyl}oxy)-5-[(phenylmethyl)oxy]benzoic acid

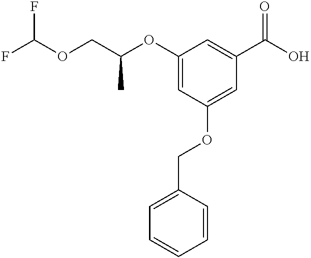

Lithium hydroxide monohydrate (19 mg, 0.45 mmol) in water (2 mL) was added to methyl 3-({(1S)-2-[(difluoromethyl)oxy]-1-methylethyl}oxy)-5-[(phenylmethyl)oxy]benzoate (0.11 g, 0.3 mmol) in THF (4 mL) and the mixture stirred at RT for 20 hours. The THF was removed in vacuo and the aqueous layer adjusted to pH 3 with citric acid then extracted into ethyl acetate (2×30 mL). The organics were washed with water (30 mL), brine (30 mL), dried (MgSO₄), filtered and the solvent removed in vacuo to give the desired compound (0.1 g). ¹H NMR δ (d₆-DMSO): 1.27 (d, 3H), 4.00 (m, 2H), 4.75 (sextet, 1H), 5.15 (s, 2H), 6.72 (t, 1H), 7.08 (t, 1H), 7.16 (t, 1H), 7.41 (m, 5H), 12.95 (s, 1H). m/z 351 (M+H)⁺.

### Methyl 3-({(1S)-2-[(difluoromethyl)oxy]-1-methylethyl}oxy)-5-[(phenylmethyl)oxy]benzoate

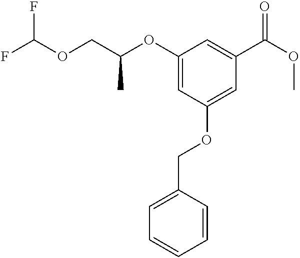

2-(Fluorosulphonyl)difluoroacetic acid (0.239 mL, 2.31 mmol) was added dropwise, with stirring, to a degassed mixture of methyl 3-{[(1S)-2-hydroxy-1-methylethyl]oxy}-5-[(phenylmethyl)oxy]benzoate (0.73 g, 2.31 mmol) and copper (I) iodide (88 mg, 0.46 mmol) in acetonitrile (10 mL) at 45° C. The reaction was stirred at 45° C. for 24 hours. The solvent was removed in vacuo and ethyl acetate (30 mL) added. The organics were washed with water (30 mL), brine (30 mL), dried (MgSO₄), filtered and the solvent removed in vacuo to give a yellow oil which was chromatographed on silica, eluting with a gradient of 0-30% ethyl acetate in isohexane, to give the desired compound (0.11 g).

¹H NMR δ (CDCl₃): 1.37 (d, 3H), 3.93 (s, 3H), 4.00 (m, 2H), 4.63 (sextet, 1H), 5.10 (s, 2H), 6.28 (t, 1H), 6.77 (t, 1H), 7.28 (t, 1H), 7.41 (m, 6H). m/z 367 (M+H)⁺.

### Methyl 3-{[(1S)-2-hydroxy-1-methylethyl]oxy}-5-[(phenylmethyl)oxy]benzoate

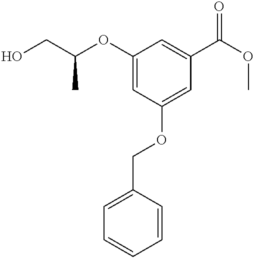

Benzyl bromide (1.89 g, 7.20 mmol) was added to a mixture of methyl 3-hydroxy-5-[(1S)-2-hydroxy-1-methylethoxy]benzoate (1.55 g, 6.86 mmol) and potassium carbonate (1.89 g, 0.014 mol) in DMF (16 mL) and the reaction stirred at RT for 20 hours. Ethyl acetate (40 mL) was added and the mixture washed with water (40 mL), saturated sodium bicarbonate solution (40 mL), brine (40 mL), dried (MgSO4), filtered and the solvent removed in vacuo to give a red oil which was chromatographed on silica, eluting with a gradient of 0-100% ethyl acetate in isohexane, to give the desired compound (1.7 g).

¹H NMR δ (CDCl₃): 1.30 (d, 3H), 1.95 (m, 1H), 3.76 (m, 2H), 3.92 (s, 3H), 4.53 (m, 1H), 5.11 (s, 2H), 6.78 (t, 1H), 7.25 (m, 1H), 7.32 (m, 1H), 7.45 (m, 5H). m/z 317 (M+H)⁺.

### Methyl 3-hydroxy-5-[(1S)-2-hydroxy-1-methylethoxy]benzoate

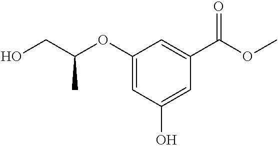

Trimethylsilyl iodide (115 mL, 0.79 mol) was added to a solution of methyl 3-hydroxy-5-[(1S)-2-methoxy-(1-methylethyl)oxy]benzoate (38.01 g, 0.158 mol) in acetonitrile (500 mL) and stirred for 24 hours. Methanol (300 mL) was added and the reaction stirred for 10 mins. 10% w/v Aqueous sodium thiosulfate pentahydrate (100 mL) was added to the mixture and stirred for 20 mins. The reaction mixture was neutralised with saturated aqueous sodium bicarbonate solution, the organic solvents removed in vacuo, and the product extracted into ethyl acetate (4×100 mL). The combined organic layers were dried (MgSO₄), filtered and the solvents removed in vacuo. The crude material was crystallised from ethyl acetate to give the title compound (16.8 g)

¹H NMR δ (d₆-DMSO): 1.18 (d, 3H), 3.40-3.55 (m, 2H), 3.80 (s, 3H), 4.35 (sex, 1H), 4.80 (t, 1H), 6.57 (m, 1H), 6.90 (m, 2H), 9.75 (s, 1H). m/z 304 (M+H)⁺

### Methyl 3-Hydroxy-5-[(1S)-2-methoxy-(1-methylethyl)oxy]benzoate

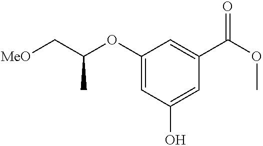

Methyl 3-[(1S)-2-methoxy-(1-methylethyl)oxy]-5-{[phenylmethyl]oxy}benzoate (50.0 g, 0.152 mmol) was dissolved in a mixture of THF:ethanol (600 mL) and the flask evacuated and purged with nitrogen (3 times). 10% Palladium on carbon (5.0 g) was added and the flask further evacuated and finally purged with hydrogen gas. The reaction mixture was stirred at ambient temperature for 20 hours until completion. The reaction mixture was evacuated and purged with nitrogen (3 times). The catalyst was filtered off, and the filtrate concentrated in vacuo to give the desired compound (36.7 g).

¹H NMR δ (d₆-DMSO): 1.2 (d, 3H), 3.25 (s, 3H), 3.44 (m, 2H), 3.82 (s, 3H), 4.55 (m, 1H), 6.6 (s, 1H), 6.9 (s, 1H), 6.95 (s, 1H), 9.8 (s, 1H).

The preparation of methyl 3-[(1S)-2-methoxy-(1-methylethyl)oxy]-5-{[phenylmethyl]oxy}benzoate was described previously.

### Biological

### Tests:

The biological effects of the compounds of formula (I) may be tested in the following way:

### (1) Enzymatic Activity

Enzymatic activity of recombinant human pancreatic GLK may be measured by incubating GLK, ATP and glucose. The rate of product formation may be determined by coupling the assay to a G-6-P dehydrogenase, NADP/NADPH system and measuring the linear increase with time of optical density at 340 nm (Matschinsky et al 1993). Activation of GLK by compounds can be assessed using this assay in the presence or absence of GLKRP as described in Brocklehurst et al (Diabetes 2004, 53, 535-541).

### Production of Recombinant GLK and GLKRP:

Human GLK and GLKRP cDNA was obtained by PCR from human pancreatic and hepatic mRNA respectively, using established techniques described in Sambrook J, Fritsch E F & Maniatis T, 1989. PCR primers were designed according to the GLK and GLKRP cDNA sequences shown in Tanizawa et al 1991 and Bonthron, D. T. et al 1994 (later corrected in Warner, J. P. 1995).

### Cloning in Bluescript II Vectors

GLK and GLKRP cDNA was cloned in E. coli using pBluescript II, (Short et al 1998) a recombinant cloning vector system similar to that employed by Yanisch-Perron C et al (1985), comprising a colEI-based replicon bearing a polylinker DNA fragment containing multiple unique restriction sites, flanked by bacteriophage T3 and T7 promoter sequences; a filamentous phage origin of replication and an ampicillin drug resistance marker gene.

### Transformations

E. coli transformations were generally carried out by electroporation. 400 mL, cultures of strains DH5a or BL21(DE3) were grown in L-broth to an OD 600 of 0.5 and harvested by centrifugation at 2,000 g. The cells were washed twice in ice-cold deionised water, resuspended in 1 mL 10% glycerol and stored in aliquots at −70° C. Ligation mixes were desalted using Millipore V Series™ membranes (0.0025 mm) pore size). 40 mL of cells were incubated with 1 mL of ligation mix or plasmid DNA on ice for 10 minutes in 0.2 cm electroporation cuvettes, and then pulsed using a Gene Pulser™ apparatus (BioRad) at 0.5 kVcm⁻¹, 250 mF. Transformants were selected on L-agar supplemented with tetracycline at 10 mg/mL or ampicillin at 100 mg/mL.

### Expression

GLK was expressed from the vector pTB375NBSE in E. coli BL21 cells, producing a recombinant protein containing a 6-His tag immediately adjacent to the N-terminal methionine. Alternatively, another suitable vector is pET21(+)DNA, Novagen, Cat number 697703. The 6-His tag was used to allow purification of the recombinant protein on a column packed with nickel-nitrilotriacetic acid agarose purchased from Qiagen (cat no 30250).

GLKRP was expressed from the vector pFLAG CTC (IBI Kodak) in E. coli BL21 cells, producing a recombinant protein containing a C-terminal FLAG tag. The protein was purified initially by DEAE Sepharose ion exchange followed by utilisation of the FLAG tag for final purification on an M2 anti-FLAG immunoaffinity column purchased from Sigma-Aldrich (cat no. A1205).

### (2) Oral Glucose Tolerance Test (OGTT)

Oral glucose tolerance tests were done on conscious Zucker obese fa/fa rats (age 12-13 weeks or older) fed a high fat diet (45% kcal fat) for at least two weeks prior to experimentation. The animals were fasted for 2 hours before use for experiments. A test compound or a vehicle was given orally 120 minutes before oral administration of a glucose solution at a dose of 2 g/kg body weight. Blood glucose levels were measured using a Accucheck glucometer from tail bled samples taken at different time points before and after administration of glucose (time course of 60 minutes). A time curve of the blood glucose levels was generated and the area-under-the-curve (AUC) for 120 minutes was calculated (the time of glucose administration being time zero). Percent reduction in glucose excursion was determined using the AUC in the vehicle-control group as zero percent reduction.

Compounds of the invention generally have an activating activity for glucokinase with an EC₅₀ of less than about 500 nM. For example, Example 1 has an EC₅₀ of 88 nm and an activity of 50% in OGTT at 10 mg/kg.

### REFERENCES

1 Printz, R. L., Magnuson, M. A. and Granner, D. K. (1993) Annual Review of Nutrition 13, 463-96
2 DeFronzo, R. A. (1988) Diabetes 37, 667-87
3 Froguel, P., Zouali, H., Vionnet, N., Velho, G., Vaxillaire, M., Sun, F., Lesage, S., Stoffel, M., Takeda, J. and Passa, P. (1993) New England Journal of Medicine 328, 697-702
4 Bell, G. I., Pilkis, S. J., Weber, I. T. and Polonsky, K. S. (1996) Annual Review of Physiology 58, 171-86
5 Velho, G., Petersen, K. F., Perseghin, G., Hwang, J. H., Rothman, D. L., Pueyo, M. E., Cline, G. W., Froguel, P. and Shulman, G. I. (1996) Journal of Clinical Investigation 98, 1755-61
6 Christesen, H. B., Jacobsen, B. B., Odili, S., Buettger, C., Cuesta-Munoz, A., Hansen, T., Brusgaard, K., Massa, O., Magnuson, M. A., Shiota, C., Matschinsky, F. M. and Barbetti, F. (2002) Diabetes 51, 1240-6
6a Gloyn, A. L., Noordam, K., Willemsen, M. A. A. P., Ellard, S., Lam, W. W. K., Campbell, I. W., Midgley, P., Shiota, C., Buettger, C., Magnuson, M. A., Matschinsky, F. M., and Hattersley, A. T.; Diabetes 52: 2433-2440
7 Glaser, B., Kesavan, P., Heyman, M., Davis, E., Cuesta, A., Buchs, A., Stanley, C. A., Thornton, P. S., Permutt, M. A., Matschinsky, F. M. and Herold, K. C. (1998) New England Journal of Medicine 338, 226-30
8 Caro, J. F., Triester, S., Patel, V. K., Tapscott, E. B., Frazier, N. L. and Dohm, G. L. (1995) Hormone & Metabolic Research 27, 19-22
9 Desai, U. J., Slosberg, E. D., Boettcher, B. R., Caplan, S. L., Fanelli, B., Stephan, Z., Gunther, V. J., Kaleko, M. and Connelly, S. (2001) Diabetes 50, 2287-95
10 Shiota, M., Postic, C., Fujimoto, Y., Jetton, T. L., Dixon, K., Pan, D., Grimsby, J., Grippo, J. F., Magnuson, M. A. and Chemington, A. D. (2001) Diabetes 50, 622-9
11 Ferre, T., Pujol, A., Riu, E., Bosch, F. and Valera, A. (1996) Proceedings of the National Academy of Sciences of the United States of America 93, 7225-30
12 Seoane, J., Barbera, A., Telemaque-Potts, S., Newgard, C. B. and Guinovart, J. J. (1999) Journal of Biological Chemistry 274, 31833-8
13 Moore, M. C., Davis, S. N., Mann, S. L. and Chemington, A. D. (2001) Diabetes Care 24, 1882-7
14 Alvarez, E., Roncero, I., Chowen, J. A., Vazquez, P. and Blazquez, E. (2002) Journal of Neurochemistry 80, 45-53
15 Lynch, R. M., Tompkins, L. S., Brooks, H. L., Dunn-Meynell, A. A. and Levin, B. E. (2000) Diabetes 49, 693-700
16 Roncero, I., Alvarez, E., Vazquez, P. and Blazquez, E. (2000) Journal of Neurochemistry 74, 1848-57
17 Yang, X. J., Kow, L. M., Funabashi, T. and Mobbs, C. V. (1999) Diabetes 48, 1763-1772
18 Schuit, F. C., Huypens, P., Heimberg, H. and Pipeleers, D. G. (2001) Diabetes 50, 1-11
19 Levin, B. E. (2001) International Journal of Obesity 25, supplement 5, S68-S72.
20 Alvarez, E., Roncero, I., Chowen, J. A., Thorens, B. and Blazquez, E. (1996) Journal of Neurochemistry 66, 920-7
21 Mobbs, C. V., Kow, L. M. and Yang, X. J. (2001) American Journal of Physiology-Endocrinology & Metabolism 281, E649-54
22 Levin, B. E., Dunn-Meynell, A. A. and Routh, V. H. (1999) American Journal of Physiology 276, R1223-31
23 Spanswick, D., Smith, M. A., Groppi, V. E., Logan, S. D. and Ashford, M. L. (1997) Nature 390, 521-5
24 Spanswick, D., Smith, M. A., Mirshamsi, S., Routh, V. H. and Ashford, M. L. (2000) Nature Neuroscience 3, 757-8
25 Levin, B. E. and Dunn-Meynell, A. A. (1997) Brain Research 776, 146-53
26 Levin, B. E., Govek, E. K. and Dunn-Meynell, A. A. (1998) Brain Research 808, 317-9
27 Levin, B. E., Brown, K. L. and Dunn-Meynell, A. A. (1996) Brain Research 739, 293-300
28 Rowe, I. C., Boden, P. R. and Ashford, M. L. (1996) Journal of Physiology 497, 365-77
29 Fujimoto, K., Sakata, T., Arase, K., Kurata, K., Okabe, Y. and Shiraishi, T. (1985) Life Sciences 37, 2475-82
30 Kurata, K., Fujimoto, K. and Sakata, T. (1989) Metabolism: Clinical & Experimental 38, 46-51
31 Kurata, K., Fujimoto, K., Sakata, T., Etou, H. and Fukagawa, K. (1986) Physiology & Behavior 37, 615-20
32 Jetton T. L., Liang Y., Pettepher C. C., Zimmerman E. C., Cox F. G., Horvath K., Matschinsky F. M., and Magnuson M. A., J. Biol. Chem., February 1994; 269: 3641-3654
33 Reimann F. and Gribble F. M., Diabetes 2002 51: 2757-2763
34 Cheung A. T., Dayanandan B., Lewis J. T., Korbutt G. S., Rajotte R. V., Bryer-Ash M., Boylan M. O., Wolfe M. M., Kieffer T. J., Science, Vol 290, Issue 5498, 1959-1962, 8 Dec. 2000

1-18. (canceled)

19. A compound of Formula (I) or a salt thereof, 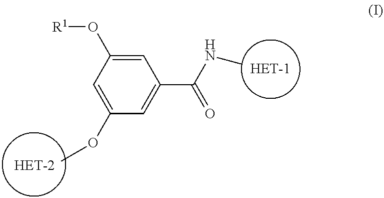
wherein:
R¹ is selected from isopropyl, but-2-yl, 1,1,1-trifluoroprop-2-yl, 1,3-difluoroprop-2-yl, but-1-yn-3-yl, 1-hydroxyprop-2-yl, 2-hydroxybut-3-yl, 1-hydroxybut-2-yl, tetrahydrofuryl, tetrahydropyranyl, 1-methoxyprop-2-yl, 1-methoxybut-2-yl, 2-hydroxyprop-1-yl, 2-methoxyprop-1-yl, 2-hydroxybut-1-yl, 2-methoxybut-1-yl, 1-fluoromethoxyprop-2-yl, 1,1-difluoromethoxyprop-2-yl, and 1-trifluoromethoxyprop-2-yl;
HET-1 is a 5- or 6-membered, C-linked heteroaryl ring containing a nitrogen atom in the 2-position and optionally 1 or 2 further ring heteroatoms independently selected from O, N, and S; which ring is optionally substituted on any nitrogen atom by a substituent selected from R⁷ and/or on any available carbon atom by 1 or 2 substituents independently selected from R⁶;
HET-2 is a heterocyclic ring system comprising a Ring A which is bonded to the linking ether oxygen, and a Ring B which is fused to Ring A; wherein Ring A is a 5- or 6-membered heteroaryl ring, and Ring A is optionally substituted with a substituent selected from R⁴; Ring B is phenyl or Ring B is a 5- to 7-membered heterocyclic ring, containing 1, 2, or 3 ring hetereoatoms independently selected from O, S, and N, provided that there are no O—O, S—O, or S—S bonds within the ring; wherein any ring carbon or sulfur atom may optionally be oxidised; and wherein Ring B is optionally substituted on any nitrogen atom with a substituent selected from R² and/or on any available carbon atom with 1 or 2 substituents independently selected from R³;
R² is selected from (1-4C)alkyl, (3-6C)cycloalkyl, benzyl, (1-4C)alkylcarbonyl, (1-4C)alkylsulphonyl, hydroxy(1-4C)alkyl and (1-4C)alkoxy(1-4C)alkyl;
R³ is selected from (1-4C)alkyl, (3-6C)cycloalkyl, (1-4C)alkoxy, hydroxy, fluoro and chloro;
when R⁴ is a substituent on carbon, it is selected from fluoro and chloro;
when R⁴ is a substituent on nitrogen it is selected from (1-4C)alkyl, (3-6C)cycloalkyl, benzyl, (1-4C)alkylcarbonyl, (1-4C)alkylsulphonyl, hydroxy(1-4C)alkyl, and (1-4C)alkoxy(1-4C)alkyl;
R⁶ is independently selected from (1-4C)alkyl, halo, hydroxy(1-4C)alkyl, (1-4C)alkoxy(1-4C)alkyl, (1-4C)alkylS(O)ₚ(1-4C)alkyl, amino(1-4C)alkyl, (1-4C)alkylamino(1-4C)alkyl, and di(1-4C)alkylamino(1-4C)alkyl;
R⁷ is independently selected from (1-4C)alkyl, hydroxy(1-4C)alkyl, (1-4C)alkoxy(1-4C)alkyl, (1-4C)alkylS(O)ₚ(1-4C)alkyl, amino(1-4C)alkyl, (1-4C)alkylamino(1-4C)alkyl, and di(1-4C)alkylamino(1-4C)alkyl; and
p is independently 0, 1 or 2.

20. A compound of Formula (I) as claimed in claim 19 or a salt thereof wherein R¹ is of sub-formula X: 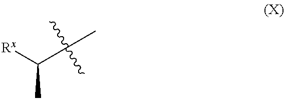
wherein R^{x} is selected from methyl, ethyl, trifluoromethyl, ethynyl, hydroxymethyl, hydroxyethyl, methoxymethyl, fluoromethoxymethyl, difluoromethoxymethyl, and trifluoromethoxymethyl.

21. A compound of Formula (I) as claimed in claim 19 or a salt thereof wherein R¹ is 1-hydroxyprop-2-yl.

22. A compound of Formula (I) as claimed in claim 19 or a salt thereof, wherein HET-1 is a 5-membered ring.

23. A compound of Formula (I) as claimed in claim 19, or a salt thereof, wherein HET-1 is N-methylpyrazolyl or methylpyrazinyl.

24. A compound of Formula (I) as claimed in claim 19, or a salt thereof, wherein HET-2 is selected from Formulae A to F; 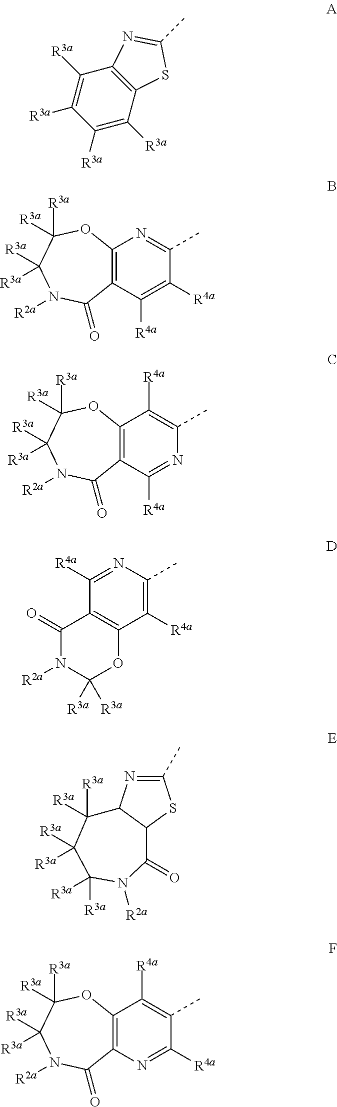
wherein each R^{2a} is independently hydrogen or is selected from R²;
each R^{3a} is independently hydrogen or is selected from R³; and
each R^{4a} is independently hydrogen or is selected from R⁴.

25. A compound as claimed in claim 24 or a salt thereof,
wherein each R^{2a} is hydrogen or methyl;
each R^{3a} is hydrogen or methyl; and
each R^{4a} is hydrogen, chloro, or fluoro.

26. A compound of Formula (I) as claimed in claim 19, or a salt thereof, wherein HET-2 is selected from Formulae H to P as follows:

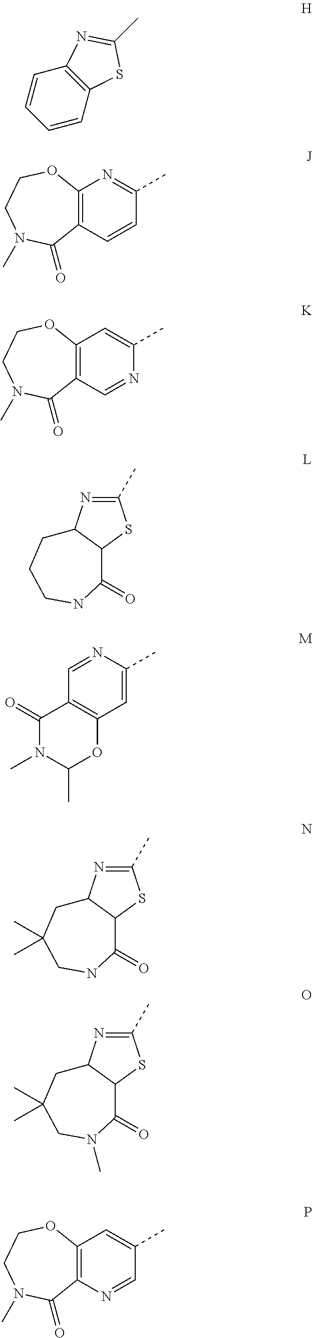

27. A compound of Formula (I) as claimed in claim 18 which is any one or more of the following:
3-(1,3-benzothiazol-2-yloxy)-5-{[(1S)-2-hydroxy-1-methylethyl]oxy}-N-(1-methyl-1H-pyrazol-3-yl)benzamide;
3-{[2-fluoro-1-(fluoromethyl)ethyl]oxy}-5-[(4-methyl-5-oxo-2,3,4,5-tetrahydropyrido[3,2-f][1,4]oxazepin-8-yl)oxy]-N-(1-methyl-1H-pyrazol-3-yl)benzamide;
3-[(7-fluoro-4-methyl-5-oxo-2,3,4,5-tetrahydropyrido[3,2-f][1,4]oxazepin-8-yl)oxy]-5-[(1-methylethyl)oxy]-N-(1-methyl-1H-pyrazol-3-yl)benzamide;
3-[(7-fluoro-4-methyl-5-oxo-2,3,4,5-tetrahydropyrido[3,2-f][1,4]oxazepin-8-yl)oxy]-5-{[(1S)-1-methyl-2-(methyloxy)ethyl]oxy}-N-(1-methyl-1H-pyrazol-3-yl)benzamide;
3-[(1-methylethyl)oxy]-5-[(4-methyl-5-oxo-2,3,4,5-tetrahydropyrido[3,4-f][1,4]oxazepin-8-yl)oxy]-N-(1-methyl-1H-pyrazol-3-yl)benzamide;
3-{[(1S)-1-methyl-2-(methyloxy)ethyl]oxy}-N-(1-methyl-1H-pyrazol-3-yl)-5-[(4-oxo-5,6,7,8-tetrahydro-4H-[1,3]thiazolo[5,4-c]azepin-2-yl)oxy]benzamide; and
3-[(1-methylethyl)oxy]-N-(1-methyl-1H-pyrazol-3-yl)-5-[(4-oxo-5,6,7,8-tetrahydro-4H-[1,3]thiazolo[5,4-c]azepin-2-yl)oxy]benzamide; and/or
3-{[(1S)-1-methyl-2-(methyloxy)ethyl]oxy}-5-[(4-methyl-5-oxo-2,3,4,5-tetrahydropyrido[3,4-f][1,4]oxazepin-8-yl)oxy]-N-(1-methyl-1H-pyrazol-3-yl)benzamide;
3-[(2,3-dimethyl-4-oxo-3,4-dihydro-2H-pyrido[3,4-e][1,3]oxazin-7-yl)oxy]-5-{[(1S)-1-methyl-2-(methyloxy)ethyl]oxy}-N-(1-methyl-1H-pyrazol-3-yl)benzamide;
3-[(7,7-dimethyl-4-oxo-5,6,7,8-tetrahydro-4H-[1,3]thiazolo[5,4-c]azepin-2-yl)oxy]-5-{[(1S)-1-methyl-2-(methyloxy)ethyl]oxy}-N-(1-methyl-1H-pyrazol-3-yl)benzamide;
3-[(1-methylethyl)oxy]-N-(1-methyl-1H-pyrazol-3-yl)-5-[(5,7,7-trimethyl-4-oxo-5,6,7,8-tetrahydro-4H-[1,3]thiazolo[5,4-c]azepin-2-yl)oxy]benzamide;
3-{[(1S)-1-methyl-2-(methyloxy)ethyl]oxy}-N-(1-methyl-1H-pyrazol-3-yl)-5-[(5,7,7-trimethyl-4-oxo-5,6,7,8-tetrahydro-4H-[1,3]thiazolo[5,4-c]azepin-2-yl)oxy]benzamide;
3-[(1-methylethyl)oxy]-5-[(5-methyl-4-oxo-5,6,7,8-tetrahydro-4H-[1,3]thiazolo[5,4-c]azepin-2-yl)oxy]-N-(1-methyl-1H-pyrazol-3-yl)benzamide;
3-{[(1S)-1-methyl-2-(methyloxy)ethyl]oxy}-5-[(5-methyl-4-oxo-5,6,7,8-tetrahydro-4H-[1,3]thiazolo[5,4-c]azepin-2-yl)oxy]-N-(1-methyl-1H-pyrazol-3-yl)benzamide;
3-{[(1S)-1-methyl-2-(methyloxy)ethyl]oxy}-5-[(4-methyl-5-oxo-2,3,4,5-tetrahydropyrido[2,3-f][1,4]oxazepin-8-yl)oxy]-N-(5-methylpyrazin-2-yl)benzamide;
N-(1-methyl-1H-pyrazol-3-yl)-3-[(3S)-tetrahydrofuran-3-yloxy]-5-[(5,7,7-trimethyl-4-oxo-5,6,7,8-tetrahydro-4H-[1,3]thiazolo[5,4-c]azepin-2-yl)oxy]benzamide; and/or
3-({(1S)-2-[(difluoromethyl)oxy]-1-methylethyl}oxy)-5-[(4-methyl-5-oxo-2,3,4,5-tetrahydropyrido[3,4-f][1,4]oxazepin-8-yl)oxy]-N-(1-methyl-1H-pyrazol-3-yl)benzamide;
or a salt thereof.

28. A pharmaceutical composition comprising a compound according to claim 19, or a pharmaceutically-acceptable salt thereof, together with a pharmaceutically acceptable diluent or carrier.

29. A method of treating GLK mediated diseases comprising administering an effective amount of a compound of Formula (I) as claimed in claim 19 or a pharmaceutically-acceptable salt thereof, to a mammal in need of such treatment.

30. The method of claim 29, wherein the GLK mediated disease is type 2 diabetes.

31. A process for the preparation of a compound of Formula (I) as claimed in claim 19, comprising:
(a) reacting an acid of Formula (III) or activated derivative thereof with a compound of Formula (IV), wherein R¹ is as defined for Formula (I) or a protected version thereof; 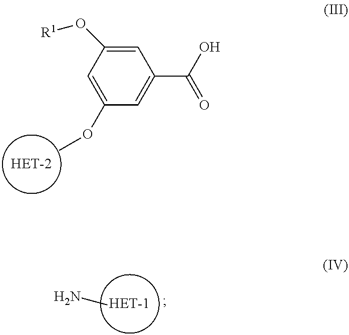
or
(b) reacting a compound of Formula (V) with a compound of Formula (VI), 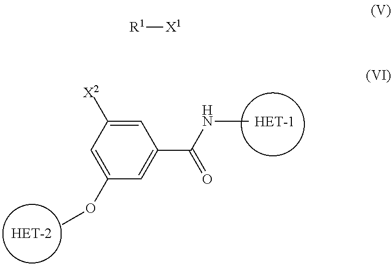
wherein X¹ is a leaving group and X² is a hydroxyl group, or X¹ is a hydroxyl group and X² is a leaving group; and wherein R¹ is as defined for Formula (I) or a protected version thereof;
or
reacting a compound of Formula (V) with the intermediate ester of Formula (VII), wherein P¹ is a protecting group, followed by ester hydrolysis and amide formation; 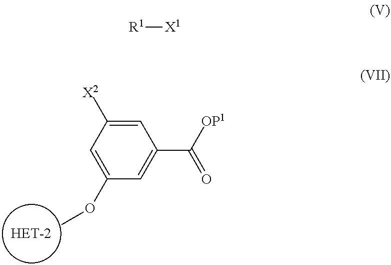
or
(c) reacting a compound of Formula (VIII) with a compound of Formula (IX) 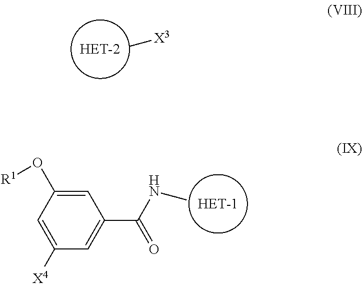
wherein X³ is a leaving group or an organometallic reagent and X⁴ is a hydroxyl group; or X³ is a hydroxyl group and X⁴ is a leaving group or an organometallic reagent; and wherein R¹ is as defined for Formula (I) or a protected version thereof;
or
reacting a compound of Formula (VIII) with the intermediate ester Formula (X), followed by ester hydrolysis and amide formation; 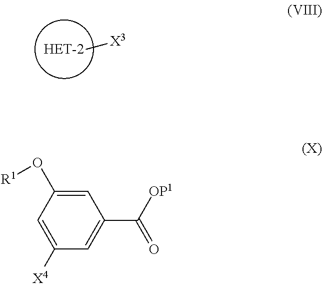
or
(d) reacting a compound of Formula (XI) with a compound of Formula (XII), 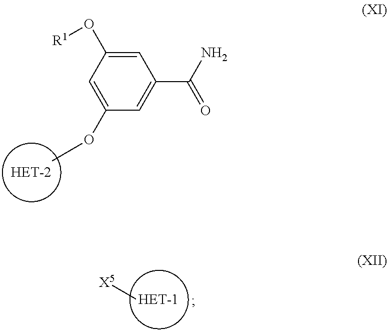
wherein X⁵ is a leaving group; and wherein R¹ is as defined for Formula (I) or a protected version thereof;
or
cyclising a compound of Formula (XIII) to a compound of Formula (I) 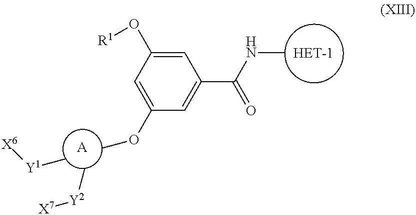
wherein Y¹ and Y² are 0 to 4 atom linkers attached to adjacent atoms in ring A; wherein each linker atom is independently selected from C, N, S, or O; wherein any C or S can be optionally oxidised and any atom can be optionally substituted provided it is not quaternised and there are no S—S or O—O bonds; X⁶ can be any nucleophilic species and X⁷ a leaving group or vice versa; and wherein R¹ is as defined in Formula (I) or a protected version thereof;
or
cyclising an intermediate ester of Formula (XIV) to a compound of Formula (I), followed by ester hydrolysis and amide formation; 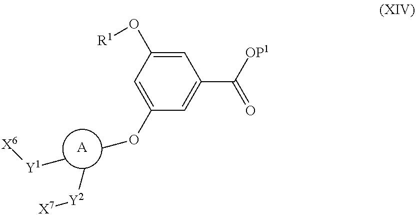
and thereafter, optionally:
i) converting a compound of Formula (I) into another compound of Formula (I);
ii) removing any protecting groups; and/or
iii) forming a salt thereof.
